(19) United States
(12) Patent Application Publication
(10) Pub. No.: US 2013/0231480 A1
(43) Pub. Date: Sep. 5, 2013

(54) SEQUENCE SPECIFIC DOUBLE-STRANDED DNA/RNA BINDING COMPOUNDS AND USES THEREOF

(71) Applicants: Anwar Rayan, Kabul (IL); Mizied Falah, Romat-Haib (IL)

(72) Inventors: Anwar Rayan, Kabul (IL); Mizied Falah, Romat-Haib (IL)

(73) Assignee: GENEARREST LTD, Nazareth (IL)

(21) Appl. No.: 13/811,560

(22) PCT Filed: Jul. 21, 2011
(86) PCT No.: PCT/IL2011/000589

§ 371 Date: Apr. 5, 2013

Related U.S. Application Data
(63) Continuation of application No. 12/841,506, filed on Jul. 22, 2010.

(51) Int. Cl. C07D 473/16; C07D 473/18

(52) U.S. Cl. CPC C07D 473/16; C07D 473/18
USPC 544/276; 544/277

(57) ABSTRACT

The present invention provides specific double-stranded DNA/RNA binding compounds having a polymeric structure, which are in fact, triplex forming molecules capable of binding tightly and specifically to predetermined sequences in the major groove of double stranded nucleic acid molecules; as well as pharmaceutical compositions comprising thereof. The triplex forming molecules and the pharmaceutical compositions of the invention can be used for various therapeutic applications such as site-specific modulation of gene expression, targeting of DNA or RNA damage, and gene knockout, as well as for diagnostic applications in vitro.

### TECHNICAL FIELD

The present invention relates to triplex forming molecules that bind tightly and specifically to predetermined sequences in the major groove of double stranded nucleic acid molecules.

### BACKGROUND ART

Transcription of a gene gives rise to many copies of messenger RNA, which is translated into a large number of proteins. Thus, inhibition of the flow of information leading from gene to protein through targeting deoxyribonucleic acid (DNA), a strategy termed the “anti-gene strategy”, presents several advantages over inhibition at any other level. More particularly, while blocking protein or mRNA does not prevent the corresponding gene from being transcribed, interfering on gene transcription through targeting DNA is expected to bring down the mRNA concentration more efficiently and for a longer time, depending on the residence time of the anti-gene molecule on its target sequence. It should further be noted that besides using anti-gene molecules for inhibition purposes, the anti-gene strategy further enables activating gene expression through suppressing the biosynthesis of a natural repressor or by reducing termination of transcription.

To date, however, there are only few medicines on the market directed to interact with DNA, i.e. nitrogen mustards and dacarbazine that covalently react with DNA often cross-linking the strands, or bleomycin, which causes DNA breakage. Due to lacking sequence specificity, most of those compounds are highly toxic and used for chemotherapy as anticancer drugs. Groove binding by selective molecules is almost exclusively limited to the minor groove while selective recognition of the major groove has remained elusive. The design of artificial sequence specific molecules, which bind DNA specifically and stably, could provide the means to interfere in gene expression more safely.

Targeting the DNA itself so as to manipulate gene expression is thus a very attractive strategy. This approach was first contemplated about 20 years ago with the description of triplex-forming molecules (TFMs) that can bind to double-stranded DNA. The molecules currently known, which are capable of binding to double-stranded DNA with high sequence specificity and stability can be classified into (i) triplex-forming molecules, composed of either nucleotides or nucleopeptides, which bind to the oligopurines strand via the major groove of oligopyrimidine-oligopurine regions in double-stranded DNA; (ii) small molecules consisting of hairpin polyamides, which recognize short, i.e., up to seven base pairs, DNA sequences with high affinity and sequence selectivity, depending on side-by-side amino acid pairings in the minor groove; and (iii) designed zinc finger proteins, engineered to display naturally occurring zinc finger motifs as molecular building blocks in a polypeptide chain, wherein the polyfinger peptide units specifically recognize DNA triplets of the sequence XNN wherein X is G or T, and N is G, T, C or A, which have been found to be efficient in recognition of up to six such triplets.

In view of the aforesaid, it is obviously clear that all these DNA targeting molecules are not specific to certain genes since 16-18 base pairs is minimal to afford recognition of a unique target sequence. In fact, the main limitations of the triplex strategy is the need to extend the range of the recognition sequences and the design of bases that would recognize all four base pairs of DNA, i.e., A-T, T-A, C-G and G-C, upon reading of the major groove.

The use of sequence specific molecules targeted to the gene of interest may enable specifically manipulating gene expression and sequence specific molecules can thus be used in various applications such as gene-based therapeutic. For example, this technology could provide a new strategy to knockout specific genes for therapeutic purposes or function/mechanism study and might be applied in the development of new diagnostic techniques. Specific molecules capable of binding to sequences of 16-18 base pairs long might be sufficient for recognizing and binding to specifically defined sites in a genome and thus inhibiting expression of particular genes. Searching after sequence specific molecules targeting the DNA has been the center of interest of many research groups in the past two decades. Stability to nucleases, sufficient membrane penetration, sequence specificity to gene of interest and long residence time on the specific target are all crucial issues needed to be discussed when evaluating such molecules.

WO 2009/093188 discloses novel compounds that interact with the double-stranded DNA via the major groove thereof and are capable of complementarily and highly specific binding thereto via the Hoogsteen face of the double-stranded DNA helix.

### SUMMARY OF INVENTION

In one aspect, the present invention relates to a sequence specific double-stranded DNA/RNA binding compound having a polymeric structure of the general formula I:

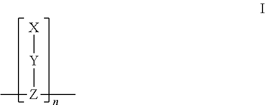

wherein

X each independently is a chemical moiety comprising a heterocyclic core capable of interacting with the A-T base pair or with the G-C base pair by forming hydrogen bonds, electrostatic interactions, or both;

Y is a covalent bond or a linker selected from —CR′₂—CO—, —CR′₂—CS—, or —(CH₂)₁₋₆— optionally substituted with at least one functional group, wherein R′ each independently is H, halogen, or a (C₁-C₃)alkyl optionally substituted with at least one functional group;

Z is a monomer selected from the formulas II, III, or IV:

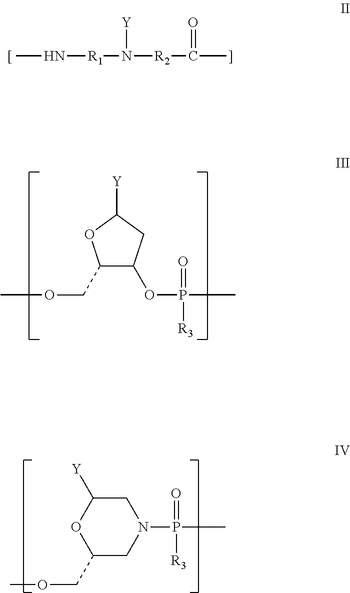

wherein

R₁ is —(CH₂)₁₋₃—, preferably —(CH₂)₁₋₂—, or R₁ together with the nitrogen atom of the secondary amine linked thereto form a 5-6-membered heterocyclic ring;

R₂ is —(CH₂)₁₋₃—, preferably —(CH₂)₁₋₂—:

R₃ is —O⁻, —OH, —OR″, —S⁻, —SH, —SR″, —NR″₂ or a (C₁-C₅)alkyl optionally substituted with at least one functional group, wherein R″ each independently is H, halogen, or a (C₁-C₅)alkyl optionally substituted with at least one functional group;

said functional group is selected from free amino, carboxyl or hydroxyl; and

n is an integer from 2 to 100,

provided that at least one of said X is not 2,6-diaminopurine-9-yl; 2-amino-6-oxopurine-9-yl: or 4-amino-2-oxo-3-pyrimidinium-1-yl.

In another aspect, the present invention relates to a monomer unit of the general formula Im:

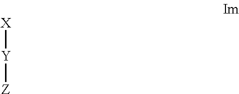

wherein

Z is a monomer of the formula IIm, IIm, or IVm:

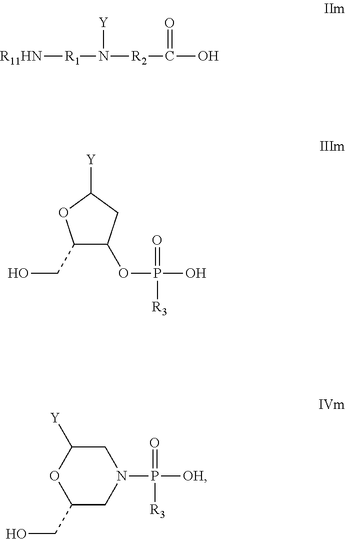

wherein R₁, R₂, R₃ and R₁₁ are as defined hereinbelow;

Y is a covalent bond or a linker selected from —CR′₂—CO—, —CR′₂—CS—, or —(CH₂)₁₋₆— optionally substituted with at least one functional group, wherein R′ each independently is H, halogen, or a (C₁-C₃)alkyl optionally substituted with at least one functional group selected from free amino, carboxyl or hydroxyl; and

X is a chemical moiety of a formula selected from the formulas X₁-X₁₃ (see Table 1) as defined hereinbelow,
but excluding the monomer units wherein Z is a monomer of the formula IIm, wherein R₁ is —(CH₂)₂—, and R₂ is —CH₂—; Y is —CR′₂—CO—; and X is 2,6-diaminopurine-9-yl, 2-amino-6-oxopurine-9-yl, 4-amino-2-oxo-3-pyrimidinium-1-yl, or an amino protected moiety of the aforesaid.

In a further aspect, the present invention provides a pharmaceutical composition comprising a sequence specific double-stranded DNA/RNA binding compound as defined above, or a pharmaceutically acceptable salt thereof, and a pharmaceutically acceptable carrier.

The binding compounds and the pharmaceutical compositions of the present invention can be used for various therapeutic applications such as site-specific modulation of gene expression and targeting of DNA or RNA damage, as well as for certain diagnostic applications in vitro.

In still a further aspect, the present invention thus provides a method of altering DNA transcription in a cell comprising exposing a double-stranded DNA in said cell to a sequence specific double-stranded DNA/RNA binding compound as defined above, or a pharmaceutically acceptable salt thereof.

In yet a further aspect, the present invention provides a method of altering gene expression in an organism comprising administering to said organism a sequence specific double-stranded DNA/RNA binding compound as defined above, or a pharmaceutically acceptable salt thereof.

### BRIEF DESCRIPTION OF DRAWINGS

FIGS. 1A-1B show the T_{M} of DNA stretches composed of 23 A-T base pairs started and terminated with C-G base pairs, without addition (1A) and after addition (1B) of the A-T selective monomer binder AH-11.

FIGS. 2A-2B show the T_{M} of DNA stretches composed of 25 C-G base pairs without addition (2A) and after addition (2B) of the A-T selective compound AH-11.

FIG. 3 shows the T_{M} of DNA stretches composed of 24 A-T base pairs without addition (a) and after addition (b) of the A-T selective compound AH-14, as (d/dT) fluorescence (465-510). The shift in T_{M} is 11.7° C.

FIG. 4 shows the T_{M} of DNA stretches composed of 25 C-G base pairs without addition (a) and after addition (b) of the A-T selective compound AH-14, as (d/dT) fluorescence (465-510). The shift in T_{M} is 0.8° C.

FIG. 5 shows the diagram produced using the ligand interactions application, demonstrating that the moiety X₁₋₁, having a pharmacophore representation of D2-A3-D4, forms hydrogen bond interactions with the A-T base pair, wherein the hydrogen bond acceptor (A3) and one of the hydrogen bond donors (D2 or D4) interact with the A base, and the other hydrogen bond donor (D2 or D4) interacts with the T base (see Example 2).

FIG. 6 shows the diagram produced using the ligand interactions application, demonstrating that the moiety X₁₋₄, having a pharmacophore representation of D2-A3-D4-D5, forms hydrogen bonds and electrostatic interactions with A-T base pair, wherein the hydrogen bond acceptor (A3), one of the hydrogen bond donors (D4) and the positively charged moiety (D)₅) interact with the A base, and the other hydrogen bond donor (D2) interacts with the T base (see Example 2).

FIG. 7 shows the diagram produced using the ligand interactions application, demonstrating that the moiety X₄₋₃, having a pharmacophore representation of A2-D3-D4-D5, forms hydrogen bonds and electrostatic interactions with G-C base pair, wherein the hydrogen bond acceptor (A2) interacts with the C base, and the hydrogen bond donors (D3 and D4) as well as the positively charged moiety (D5), interact with the G base (see Example 2).

FIG. 8 shows the diagram produced using the ligand interactions application, demonstrating that the moiety X₅₋₁, having a pharmacophore representation of A2-D)-3-D4, forms hydrogen bond interactions with the G-C base pair, wherein the hydrogen bond acceptor (A2) interacts with the C base, and the hydrogen bond donors (D3 and D4) interact with the G base (see Example 2).

FIG. 9 shows the diagram produced using the ligand interactions application, demonstrating that the moiety X₆₋₁, having a pharmacophore representation of A2-D3-D4 in acidic pH, forms hydrogen bond interactions with the G-C base pair, wherein the hydrogen bond acceptor (A2) interacts with the C base, and the hydrogen bond donors (D3 and D4) interact with the G base (see Example 2).

FIG. 10 shows the mass spectrometry (MS) spectra (ESI) of 2-(2-(2-amino-6-(benzyloxycarbonylamino)-9H-purin-9-yl)-N-(2-(tert-butoxycarbonylamino)ethyl)acetamido)acetic acid, M_{1-1b}, (pure product). RT=6.91-7.17.

FIG. 11 shows the MS spectra (ESI) of 2-(2-(6-(benzyloxycarbonylamino)-2-oxo-1H-purin-9(2H)-yl)-N-(2-(tert-butoxycarbonylamino)ethyl)acetamido)acetic acid, M_{4-1b}, (pure product). RT=6.58-6.90.

FIG. 12 shows the MS spectra (ESI) of ethyl 2-(2-(6-amino-2-oxo-1H-purin-9(2H)-yl)-N-(2-(tert-butoxycarbonylamino) ethyl)acetamido)acetate, obtained during the synthesis of 2-(2-(6-amino-2-oxo-1H-purin-9(2H)-yl)-N-(2-(tert-butoxycarbonylamino) ethyl)acetamido)acetic acid, M₄₋₂ₐ, (pure product).

FIG. 13 shows the T_{M} of DNA stretches composed of 24 A-T base pairs without addition (a) and after addition (b) of 3-(2-amino-9-(2-((2-(tert-butoxycarbonylamino)ethyl) (2-ethoxy-2-oxoethyl)amino)-2-oxoethyl)-9H-purin-6-ylamino)-N,N,N-trimethylpropan-1-aminium, an analog of the A-T selective monomer M₁₋₆ₐ, as (d/dT) fluorescence (465-510). The shift in T_{M} is 6.9° C.

FIG. 14 shows the T_{M} of DNA stretches composed of 25 C-G base pairs without addition (a) and after addition (b) of 3-(2-amino-9-(2-((2-(tert-butoxycarbonylamino)ethyl) (2-ethoxy-2-oxoethyl)amino)-2-oxoethyl)-9H-purin-6-ylamino)-N,N,N-trimethylpropan-1-aminium, an analog of the A-T selective monomer M₁₋₆ₐ, as (d/dT) fluorescence (465-510). The shift in T_{M} is <1.0° C.

FIG. 15 shows the T_{M} of DNA stretches composed of 25 C-G base pairs without addition (a) and after addition (b) of 3-(9-(2-((2-(tert-butoxycarbonylamino)ethyl)(2-ethoxy-2-oxoethyl)amino)-2-oxoethyl)-2-oxo-2,9-dihydro-1H-purin-6-ylamino)-N,N,N-trimethylpropan-1-aminium, an analog of the C-G selective monomer M₄₋₄ₐ, as (d/dT) fluorescence (465-510). The shift in T_{M} is 5.6° C.

FIG. 16 shows the T_{M} of DNA stretches composed of 24 A-T base pairs without addition (a) and after addition (b) of 3-(9-(2-((2-(tert-butoxycarbonylamino) ethyl)(2-ethoxy-2-oxoethyl)amino)-2-oxoethyl)-2-oxo-2,9-dihydro-1H-purin-6-ylamino)-N,N,N-trimethylpropan-1-aminium, an analog of the C-G selective monomer M₄₋₄ₐ, as (d/dT) fluorescence (465-510). The shift in T_{M} is 0.3° C.

FIG. 17 shows absorption (excitation) spectrum of polymer L14 measured by spectrophotometer at the range between 200 nm and 500 nm.

FIG. 18 shows fluorescence (emission) spectrum of polymer L14 (excitation wavelength of 460 nm) measured by spectrophotometer at the range between 465 nm and 800 nm.

FIG. 19 shows the T_{M} of DNA stretches composed of 25 base pairs and including the fragment GGGCC/CCCGG) without addition (a) and after addition (b) of the GGGCC-CCCGG selective binder polymer-E, as (d/dT) fluorescence (465-510). T_{M} shift from 86° C. in control well to 96° C. in treated well.

FIG. 20 shows that compound AH-1 is capable of specifically interfering with the activity of the restriction enzyme satI, in a concentration dependent manner. Un—no enzyme; “Cut 5” and “Cut 10”—different amount of PCR product without AH-13; “5+5B”, “5+5B”, “5+5B” and “5+5B”—different amount of PCR product and different amount of AH-13.

FIG. 21 shows the sequence of the chimera BCR-ABL gene synthesized according to Example 16, wherein the underlined 17-bases sequence was selected for targeting in the T_{M} test.

### DETAILED DESCRIPTION OF THE INVENTION

The present invention relates to sequence specific double-stranded DNA or RNA binding compounds, herein also identified as binding compounds or binders, having a polymeric structure of the general formula I as defined above. These binding compounds are, in fact, triplex-forming molecules capable of recognizing specific double-stranded nucleic acid molecules of up to dozens of base pairs and forming triplex structures with said double-stranded nucleic acid molecules upon binding to each of the single-strands of said sequences at each one of the base pairs of these sequences. More particularly, the binding compounds of the invention interact with the double-stranded nucleic acid molecules via the major groove and are designed to be complementary and highly specific to the hoogsteen base pair face. In sharp contrast to most of the triplex-forming molecules currently known, which interact with a single strand of the nucleic acid molecule only, the triplex-forming molecules of the present invention interact with both strands of the nucleic acid molecule and are capable of recognizing all four base pairs of the DNA, i.e., adenine-thymine (A-T), thymine-adenine (T-A), cytosine-guanine (C-G) and guanine-cytosine (G-C), as well as the adenine-uracil (A-U) and uracil-adenine (U-A) base pairs of the RNA, in which thymine is replaced by uracil.

The triplex-forming molecules of the present invention have a polymeric structure of the formula I, wherein Z is a monomer of the general formula II, III or IV forming, upon polymerization, a polyamide, a poly(2-(hydroxymethyl)tetrahydrofuran-3-yl phosphate), or a poly(2-(hydroxymethyl) morpholinophosphonic acid), respectively; Y is either a covalent bond or a linker as defined above; and X each independently is a chemical moiety comprising a heterocyclic core capable of specifically interacting with the A-T (or T-A) base pair or with the G-C (or C-G) base pair by forming hydrogen bonds and/or electrostatic interactions.

The term “polymeric structure”, as used herein with respect to the binding compound of the invention, thus means a polymeric structure having a polyamide, a poly(2-(hydroxymethyl)tetrahydrofuran-3-yl phosphate), or a poly(2-(hydroxymethyl) morpholinophosphonic acid) backbone, more particularly, a polymeric structure having the general formula I, which comprises a plurality of monomer units, each consisting of a polymerizable component Z having the general formula II, III or IV to which a chemical moiety X capable of interacting with the A-T, T-A, G-C or C-G base pair is linked via Y, which may be either a covalent bond or a linker as defined above. The monomer units composing the polymeric structure may be either identical or different forming homo-polymeric structure or hetero-polymeric structure, respectively; however, in most cases, the polymeric structure of the general formula I is a hetero-polymeric structure, the heterogeneity of which stems from the fact that monomer units comprising different X moieties compose said hetero-polymeric structure. Since each one of the chemical moieties X is capable of interacting with a different base pair, the specific moieties X linked to the polymerizable components Z in the binding compound of the invention, as well as the order of said moieties obtained upon polymerization of said monomer units, are determined according to the specific sequence of the target double-stranded nucleic acid molecule to be bound. The term “polymeric structure” as used herein encompasses dimeric, trimeric, oligomeric and polymeric structures, in which the number of the monomers Z polymerized is from 2 to 100, preferably from 5 to 75, 10 to 50, 10 to 40, or to 40, more preferably from 15 to 30, most preferably from 15 to 25.

The term “heterocyclic core”, as used herein with respect to the chemical moiety X, refers to any univalent radical of mono- or bi-cyclic ring of 5-12 atoms containing at least one carbon atom and at least one, preferably 2, 3 or 4, heteroatoms selected from nitrogen, oxygen or sulfur, which may be saturated, unsaturated, i.e., containing at least one unsaturated bond, or aromatic. Examples of such heterocyclic cores, without being limited to, include purine, dihydro-purine, imidazole, 2,3-dihydro-1H-imidazole, 2,3-dihydro-1H-imidazo[4,5-b]pyridine, dihydropyridine, dihydropyrimidine, tetrahydro-pyrimidine, 1H-pyrrole, and 1,2-dihydrooxazolol-[5,4-b]pyridine. In order to enable the chemical moiety X to efficiently interact with either the A-T or G-C base pair by forming hydrogen bonds and/or electrostatic interactions, each one of the carbon atoms of the heterocyclic core may be substituted and/or one of said carbon atoms may be double-bonded to a heteroatom selected from O, S or N, preferably O or S. In certain embodiments, one, two or three of the carbon atoms of the heterocyclic core are substituted, and/or one of said carbon atoms is double-bonded to O or S.

The term “hydrogen bond”, as used herein, refers to the interaction of a hydrogen atom with an electronegative atom such as nitrogen, oxygen, sulfur or fluorine, which can occur between molecules (intermolecular hydrogen bonding) or within different parts of a single molecule (intramolecular hydrogen bonding). The hydrogen bond is stronger than a van der Waals interaction, but weaker than covalent or ionic bonds. The term “electrostatic interaction”, as used herein, refers to any interaction occurring between charged components, molecules or ions, due to attractive forces when components of opposite electric charge are attracted to each other.

The ability of each one of the chemical moieties X to interact with a specific base pair by forming hydrogen bonds and/or electrostatic interactions results from the pharmacophore of the moiety, i.e., the set of structural features in said moiety responsible for the biological activity thereof or, more particularly, the ensemble of steric and electronic features in said moiety that enables the optimal supramolecular interactions with said base pair, thus triggering the biological activity of said moiety. In other words, the ability of a certain moiety X to interact with a certain base pair results from the structural features of that moiety, which specifically match different chemical groups with similar properties in said base pair.

The chemical moiety X of the present invention can be any moiety comprising a heterocyclic core capable of binding to double stranded nucleic acid molecule at a major groove binding site, by interacting with either the A-T or G-C base pair. More particular, these moieties have the general pharmacophore described in Schemes 1-4 hereinbelow, designed to be complementary and highly specific to the hoogsteen base pair face of a certain nucleotide base pair and optionally further capable of forming an electronic interaction with a phosphoric group of the DNA or RNA chain.

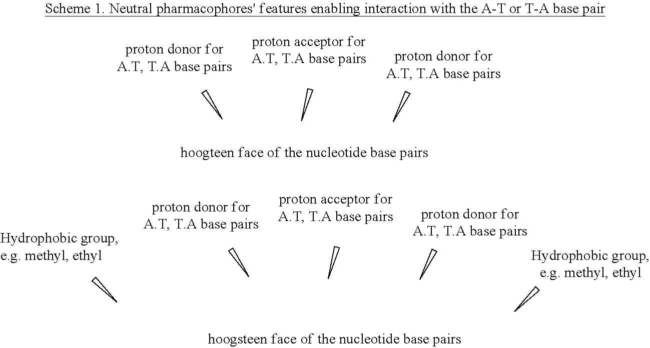

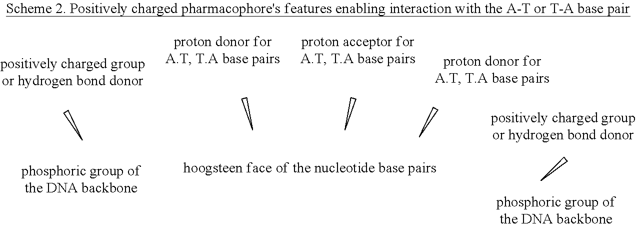

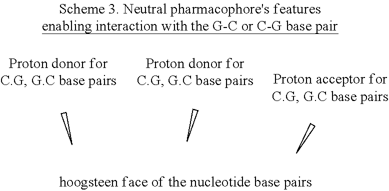

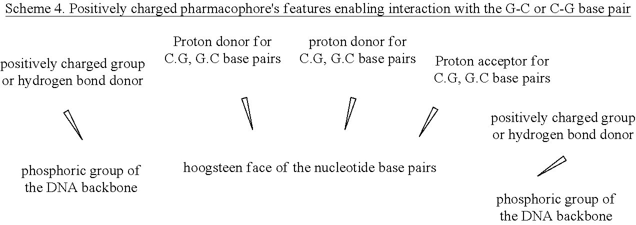

In certain embodiments, each one of the chemical moieties X in the binding compound of the invention independently has a pharmacophore representation of D1-D2-A3-D4-D5 capable of interacting with the A-T or T-A base pair by forming hydrogen bonds or electrostatic interactions, wherein D2 and D4 each independently is a hydrogen bond donor; D1 and D5 each independently is absent or selected from a hydrogen bond donor or a positively charged moiety; A3 is a hydrogen bond acceptor; the distances between the groups D2 and A3 and between the groups A3 and D4 each is about 3±1 Å; the distances between the groups D1, if present, and D2 and between the groups D5, if present, and D4 each is about 5±2 Å; the groups D2, A3 and D4 are coplanar; and the groups D1 and D5, if present, each independently is up to about 60° above or below the plane of the groups D2, A3 and D4.

In other certain embodiments, each one of the chemical moieties X has a pharmacophore representation of D1-A2-D3-D4-D5 capable of interacting with the G-C base pair by forming hydrogen bonds or electrostatic interactions, wherein D3 and D4 each independently is a hydrogen bond donor, D1 and D5 each independently is absent or selected from a hydrogen bond donor or a positively charged moiety; A2 is a hydrogen bond acceptor; the distances between the groups A2 and D3 and between the groups D3 and D4 each is about 3±1 Å; the distances between the groups D1, if present, and A2 and between the groups D5, if present, and D4 each independently is about 5±2 Å; the groups A2, D3 and D4 are coplanar, and the groups D and D5, if present, each independently is up to about 60° above or below the plane of the groups A2, D3 and D4.

The term “hydrogen bond donor”, as used herein, refers to any chemical group in which a hydrogen atom is attached to a relatively electronegative atom such as nitrogen, oxygen and fluorine. Preferred are such groups in which a hydrogen atom is attached to nitrogen, e.g., primary amines, secondary amines, primary ammonium ions, secondary ammonium ions, or tertiary ammonium ions. The term “hydrogen bond acceptor”, as used herein, refers to an electronegative atom, regardless of whether it is bonded to a hydrogen atom or not. Examples of hydrogen bond acceptors, without being limited to include N, O, S, F, Cl or Br. The term “positively charged moiety”, as used herein, means a quaternary amine.

The terms “primary amine”, “secondary amine” and “quaternary amine”, as used herein, denote the degree of substitution on nitrogen atom with organic groups, wherein in a primary amine, the nitrogen atom is linked to two hydrogen atoms and to a single organic group such as alkyl, alkenyl, alkynyl, aryl and heteroaryl; in a secondary amine, the nitrogen atom is linked to a single hydrogen atom and to two organic groups as listed above; and in a quaternary amine, the nitrogen atom is linked to four organic groups as listed above and it is positively charged. In both secondary and quaternary amines, the amine group may also be a part of a saturated, unsaturated, i.e., containing at least one unsaturated bond, or aromatic heterocyclic ring.

The terms “primary ammonium ions”, “secondary ammonium ions” and “tertiary ammonium ions”, as used herein, refer to an ammonium ion. i.e., NH₄⁺, in which one, two or three hydrogen atoms, respectively, are replaced by an organic group such as alkyl, alkenyl, alkynyl, aryl and heteroaryl. In tertiary ammonium ions, the nitrogen atom may also be a part of a saturated, unsaturated, i.e. containing at least one unsaturated bond, or aromatic heterocyclic ring, e.g. pyridazinium, pyrimidinium, pyrazinium and 1,2-dihydrooxazolo[5,4-b]pyridinium.

In particular embodiments, each one of the chemical moieties X in the binding compound of the invention independently has a pharmacophore representation of (i) D2-A3-D4, D1-D2-A3-D4. D2-A3-D4-D5 or D1-D2-A3-D4-D5, preferably D1-D2-A3-D4, D2-A3-D4-D5 or D1-D2-A3-D4-D5, capable of interacting with the A-T or T-A base pair; or (ii) A2-D3-D4, D1-A2-D3-D4, A2-D3-D4-D5 or D1-A2-D3-D4-D5, preferably D1-A2-D3-D4. A2-D3-D4-D5 or D1-A2-D3-D4-D5, capable of interacting with the G-C or C-C base pair.

In more particular embodiments, each one of the chemical moieties X in the binding compound of the invention independently is (i) a chemical moiety having a pharmacophore representation of D1-D2-A3-D4-D5 as defined above, capable of interacting with the A-T base pair, of the general formula X₁, X₂ or X₃ (see Table 1 hereinafter); or (ii) a chemical moiety having a pharmacophore representation of D1-A2-D3-D4-D5, capable of interacting with the (G-C base pair, of a general formula selected from the formulas X₄ to X₁₃ (see Table 1),

wherein

R₄ each independently is H, —COR₉ or R₉;

R₅ each independently is H, halogen, —NH₂, (C₁-C₅)alkyl optionally interrupted with a heteroatom selected from O, S or N, or —S—(C₁-C₅)alkyl;

R₆ is O or S;

R₇ is —COR₉ or R₉;

R₈ is CH or N;

R₉ is (C₁-C₃)alkyl, (C₂-C₃)alkenyl, —(CH₂)₁₋₃NHR₁₀, —(CH₂)₁₋₃N(R₁₀)₃⁺, or a 5-6-membered nitrogen containing heterocyclic ring wherein the nitrogen is optionally further substituted with a (C₁-C₃)alkyl; and

R₁₀ each independently is H or (C₁-C₃)alkyl,

wherein the asterisk (*) indicates a hydrogen bond acceptor and the bold face text indicates a hydrogen bond donor group or a positively charged moiety.

It should be understood that in cases R₄ in any one of the chemical moieties X₁-X₁₃ is —COR₉ or R₉, wherein R₉ is —(CH₂)₁₋₃NHR₁₀, the amine moiety of R₉, under physiological conditions, may be further protonated thus positively charged.

TABLE 1
Chemical moieties X of the general formulas X₁-X₁₃**
X₁ 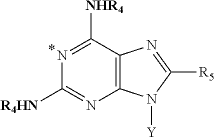
X₂ 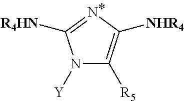
X₃ 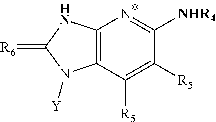
X₄ 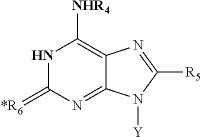
X₅ 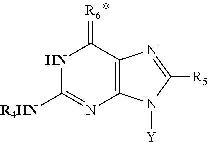
X₆ 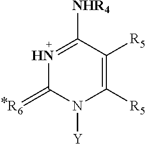
X₇ 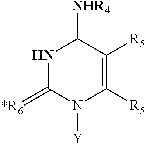
X₈ 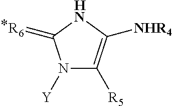
X₉ 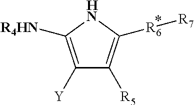
X₁₀ 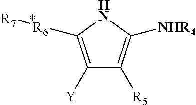
X₁₁ 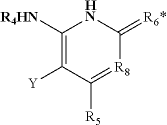
X₁₂ 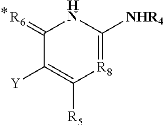
X₁₃ 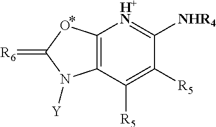
**Asterisk (*) indicates a hydrogen bond acceptor (A); and bold face text indicates a hydrogen bond donor group or a positively charged moiety (D).

The term “halogen”, as used herein, includes fluoro, chloro, bromo, and iodo, and it is preferably fluoro or chloro.

The term “alkyl” as used herein typically means a straight or branched saturated hydrocarbon radical having 1-5 carbon atoms and includes, e.g., methyl, ethyl, n-propyl, isopropyl, n-butyl, sec-butyl, isobutyl, tert-butyl, n-pentyl, 2,2-dimethylpropyl, and the like. Preferred are (C₁-C₃)alkyl groups, more preferably methyl and ethyl. The term “alkenyl” typically mean straight and branched hydrocarbon radicals having 2-3 carbon atoms and 1 double bond, and include ethenyl and propenyl.

The term “5-6-membered heterocyclic ring” denotes a monocyclic non-aromatic ring of 5-6 atoms containing at least one carbon atom and one, two or three heteroatoms selected from sulfur, oxygen or nitrogen, which may be saturated or unsaturated, i.e., containing at least one unsaturated bond. Examples of such heterocyclic rings, without being limited to, include pyrrolidine, piperidine and morpholine.

The specific chemical moieties X used in the triplex forming molecules of the present invention, which are described in the specification are herein identified as moieties X₁₋₁, X₁₋₂, X₁₋₃, X₁₋₄, X₁₋₅, X₁₋₆, X₄₋₁, X₄₋₂, X₄₋₃, X₄₋₄, X₅₋₁, X₆₋₁ and X₇₋₁, and their full chemical structures are depicted in Table 2 hereinafter.

In specific embodiments, each one of the chemical moieties X in the binding compound of the invention independently is (i) a chemical moiety of the general formula X₁, wherein R₄ of the amine group linked to the carbon atom at position 2 of the purine moiety is H; R₄ of the amine group linked to the carbon atom at position 6 of the purine moiety is H, —COCH₃ or —CO(CH₂)₂NH₂; and R₅ is H (moieties X₁₋₁, X₁₋₂ and X₁₋₃, respectively); (ii) a chemical moiety of the general formula X₁, wherein R₄ of the amine group linked to the carbon atom at position 2 of the purine moiety is —CO(CH₂)₂NH₂; R₄ of the amine group linked to the carbon atom at position 6 of the purine moiety is H; and R₅ is H (moiety X₁₋₄); (iii) a chemical moiety of the general formula X₁, wherein R₄ of the amine group linked to the carbon atom at position 2 of the purine moiety is H; R₄ of the amine group linked to the carbon atom at position 6 of the purine moiety is —(CH₂)₃NH₂ or —(CH₂)₃N(CH₃)₃⁺; and R₅ is H (moieties X₁₋₅ and X₁₋₆, respectively); (iv) a chemical moiety of the general formula X₄, wherein R₄ is H, —CO(CH₂)₂NH₂, —(CH₂)₃NH₂ or —(CH₂)₃N(CH₃)₃⁺; R₅ is H; and R₆ is O (moieties X₄₋₁, X₄₋₂, X₄₋₃ and X₄₋₄, respectively); (v) a chemical moiety of the general formula X₅, wherein R₄ is H; R₅ is H and R₆ is O (moiety X₅₋₁); (vi) a chemical moiety of the general formula X₆, wherein R₄ is H; R₅ each is H; and R₆ is O (moiety X₆₋₁); or (vii) a chemical moiety of the general formula X₇, wherein R₄ is H; R₅ each is H; and R₆ is O (moiety X₇₋₁).

TABLE 2
Specific moieties X used in the triplex forming molecules of the invention
X₁₋₁ 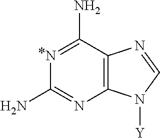
X₁₋₂ 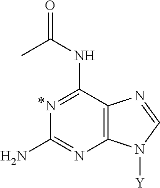
X₁₋₃ 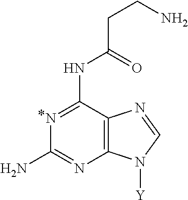
X₁₋₄ 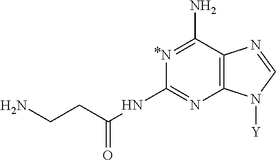
X₁₋₅ 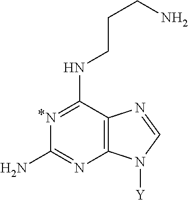
X₁₋₆ 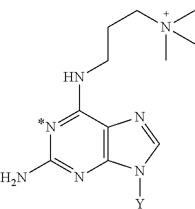
X₄₋₁ 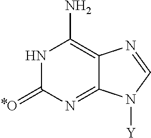
X₄₋₂ 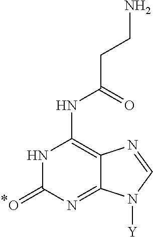
X₄₋₃ 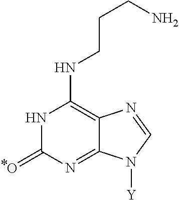
X₄₋₄ 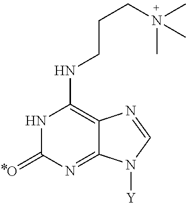
X₅₋₁ 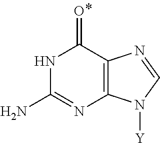
X₆₋₁ 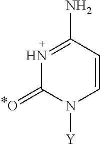
X₇₋₁

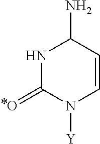

The ability of a heterocyclic core-based molecule having a pharmacophore representation of D2-A3-D4-D5 as defined above to selectively interact with the A-T base pair was first demonstrated using 3-amino-N-(6-aminopyridin-2-yl)propanamide, herein identified compound AH-11, synthesized as described in Scheme 5 hereinafter and simulating the activity of a chemical moiety X in the triplex forming molecule of the invention. As shown in Example 1, upon incubation with 25 mer A-T-DNA sequence, AH-11 increased the melting temperature (T_{M}), i.e., the temperature at which a double-stranded DNA dissociates into single strands, from 53° C. to 85° C., whereas it did not affect the T_{M} of a similar C-G-DNA. In a further study described in this Example, the ability of AH-11 to selectively interact with the A-T base pair was compared with those of similar heterocyclic core-based molecules having a pharmacophore representation of D2-A3-D4-D5 or D1-D2-A3-D4-D5 as defined above, in particular, 4-amino-N-(6-aminopyridin-2-yl)butanamide; N,N′-(pyridine-2,6-diyl)bis(3-aminopropan amide); and N,N′-(pyridine-2,6-diyl)bis(4-aminobutanamide), herein identified AH-12, AH-13 and AH-14, respectively (Scheme 5). As shown, the most effective compound among these four demonstrative compounds was AH-14, having a pharmacophore representation of D1-D2-A3-D4-D5, wherein both D1 and D5 are hydrogen bond donors (NH₂) converted under physiological conditions into positively charged moieties (NH₃⁺) capable of forming electrostatic interactions with phosphoric groups of the DNA backbone, and the distances between the groups D1 and D2 is identical to that between the groups D5 and D4, and longer than that between the groups D5 and D4 in AH-11.

In the study described in Example 2, the interactions between the pharmacophores of the moieties X₁₋₁ and X₁₋₄, capable of interacting with the A-T base pairs, and X₄₋₂, X₅₋₁ and X₆₋₁, capable of interacting with the G-C base pairs, and the Hoogsteen face of A-T base pair or G-C base pair were analyzed using the ligand interactions application (MOE 2009.10, Chemical Computing Group), which provides means to visualize an active site of a complex in diagrammatic form. As clearly shown in the diagrams produced by the ligand interactions application for the various moieties analyzed, (i) the moiety X₁₋₁, having a pharmacophore representation of D2-A3-D4, forms hydrogen bond interactions with the A-T base pair, wherein the hydrogen bond acceptor (A3) and one of the hydrogen bond donors (D2 or D4) interact with the A base, and the other hydrogen bond donor (D2 or D4) interacts with the T base; (ii) the moiety X₁₋₄, having a pharmacophore representation of D2-A3-D4-D5, forms hydrogen bonds and electrostatic interactions with A-T base pair, wherein the hydrogen bond acceptor (A3), one of the hydrogen bond donors (D4) and the positively charged moiety (D5) interact with the A base, and the other hydrogen bond donor (D2) interacts with the T base; the moiety X₄₋₂, having a pharmacophore representation of A2-D3-D4-D5, forms hydrogen bonds and electrostatic interactions with G-C base pair, wherein the hydrogen bond acceptor (A2) interacts with the C base, and the hydrogen bond donors (D3 and D4) as well as the positively charged moiety (D5), interact with the G base; and (iv) the moieties X₅₋₁ and X₆₋₁, each having a pharmacophore representation of A2-D3-D4, form hydrogen bond interactions with the G-C base pair, wherein the hydrogen bond acceptor (A2) interacts with the C base, and the hydrogen bond donors (D3 and D4) interact with the G base. As further shown, the pharmacophore area in all the moieties analyzed is more congested and less available to the solvent.

In certain embodiments, Y in the binding compound of the invention is —CR′₂—CO— or —CR′₂—CS—, wherein R′ each independently is H or a (C₁-C₂)alkyl optionally substituted with at least one functional group selected from free amino, carboxyl or hydroxyl; and Z is a monomer of the formula II as defined above, preferably wherein R₁ is —(CH₂)₂— and R₂ is —CH₂—; or R₁ is —CH₂— and R₂ is —(CH₂)₂—. In particular embodiments, Y is —CR′₂—CO—, wherein R′ each independently is H or methyl optionally substituted with at least one functional group; and Z is a monomer of the formula II, wherein either R₁ is —(CH₂)₂— and R₂ is —CH₂—, or R₁ is —CH₂— and R₂ is —(CH₂)₂—.

In other certain embodiments, Y in the binding compound of the invention is a covalent bond; and Z is a monomer of the formula III or IV. In particular embodiments, Z is a monomer of the formula III, wherein R₃ is —O⁻, —OH, —S⁻, —SiH, or a (C₁-C₂)alkyl optionally substituted with at least one functional group selected from free amino, carboxyl or hydroxyl. In other particular embodiments, Z is a monomer of the formula IV, wherein R₃ is NR″₂ wherein R″ each independently is H or a (C₁-C₂)alkyl optionally substituted with at least one functional group selected from free amino, carboxyl or hydroxyl.

In particular embodiments, the binding compound of the present invention is a compound of the general formula I, wherein each one of X independently is a chemical moiety of a general formula selected from formulas X₁-X₁₃ as defined above; Y is —CR′₂—CO— or —CR′₂—CS—, wherein R′ each independently is H or a (C₁-C₂)alkyl optionally substituted with at least one functional group; and Z is a monomer of the formula II, preferably wherein R₁ is —(CH₂)₂— and R₂ is —CH₂—; or R₁ is —CH₂— and R₂ is —(CH₂)₂—. In more particular embodiments, Y is —CR′₂—CO—, wherein R′ each independently is H or methyl optionally substituted with at least one functional group; and Z is a monomer of the formula II. In most particular embodiments, Y is —CH₂—CO—; and Z is a monomer of the formula II, wherein R₁ is —(CH₂)₂— and R₂ is —CH₂—. In certain specific embodiments, each one of X in the binding compound of the invention independently is: (i) a chemical moiety of the general formula X₁, wherein R₄ of the amine group linked to the carbon atom at position 2 of the purine moiety is H; R₄ of the amine group linked to the carbon atom at position 6 of the purine moiety is H, —COCH₃ or —CO(CH₂)₂NH₂; and R₅ is H; (ii) a chemical moiety of the general formula X₁, wherein R₄ of the amine group linked to the carbon atom at position 2 of the purine moiety is —CO(CH₂)₂NH₂; R₄ of the amine group linked to the carbon atom at position 6 of the purine moiety is H; and R₅ is H; (iii) a chemical moiety of the general formula X₁, wherein R₄ of the amine group linked to the carbon atom at position 2 of the purine moiety is H; R₄ of the amine group linked to the carbon atom at position 6 of the purine moiety is —(CH₂)₃NH₂ or —(CH₂)₃N(CH₃)₃⁺; and R₅ is H; (iv) a chemical moiety of the general formula X₄, wherein R₄ is H, —C(O(CH₂)₂NH₂, —(CH₂)₃NH₂ or —(CH₂)₃N(CH₃)₃⁺; R₅ is H; and R₆ is O; (v) a chemical moiety of the general formula X₅, wherein R₄ is H; R₅ is H; and R₆ is O; (vi) a chemical moiety of the general formula X₅, wherein R₄ is H; R₅ each is H; and R₆ is O; or (vii) a chemical moiety of the general formula X₇, wherein R₄ is H; R₅ each is H; and R₆ is O.

As stated above, the triplex forming molecules of the present invention have either homo- or hetero-polymeric structure, wherein monomers of the general formula II, III or IV represented by Z in the general formula I, to each of which a chemical moiety X capable of interacting with the A-T or G-C base pair is linked either covalently or via a linker, are polymerized. In other words, in order to prepare the triplex forming molecules of the invention, monomer units consisting of the components X, Y and Z, and capable of polymerizing to each other, are used as building blocks.

Thus, in another aspect, the present invention relates to a monomer unit of the general formula Im:

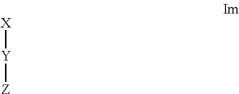

wherein

Z is a monomer of the formula IIm, IIIm, or IVm:

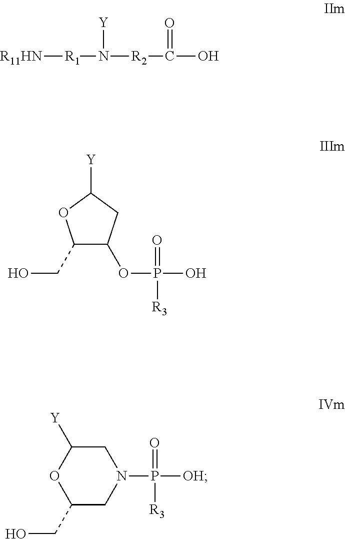

Y is a covalent bond or a linker selected from —CR′₂—CO—, —CR′₂—CS—, or —(CH₂)₁₋₆— optionally substituted with at least one functional group, wherein R′ each independently is H, halogen, or a (C₁-C₃)alkyl optionally substituted with at least one functional group; and

X is a chemical moiety of a formula selected from the formulas X₁-X₁₃ as defined above (see Table 1 hereinabove),

wherein

R₁ is —(CH₂)₁₋₃—, preferably —(CH₂)₁₋₂—, or R₁ together with the nitrogen atom of the secondary amine linked thereto form a 5-6-membered heterocyclic ring;

R₂ is —(CH₂)₁₋₃—, preferably —(CH₂)₁₋₂—;

R₃ is —O⁻, —OH, —OR″, —S⁻, —SH, —SR″, —NR″₂ or a (C₁-C₅)alkyl optionally substituted with at least one functional group, wherein R″ each independently is H, halogen, or a (C₁-(C₅)alkyl optionally substituted with at least one functional group;

R₄ each independently is —COR₉, R₉ or R₁₁;

R₅ each independently is H, halogen, —NH₂, (C₁-C₅)alkyl optionally interrupted with a heteroatom selected from O, S or N, or —S—(C₁-C₅)alkyl;

R₆ is O or S;

R₇ is —COR₉ or R₉;

R₈ is CH or N;

R₉ is (C₁-C₃)alkyl, (C₂-C₃)alkenyl, —(CH₂)₁₋₃NHR₁₀, —(CH₂)₁₋₃N(R₁₀)₃⁺, or a 5-6-membered nitrogen containing heterocyclic ring wherein the nitrogen is optionally further substituted with a (C₁-C₃)alkyl;

R₁₀ each independently is a (C₁-C₃)alkyl or R₁₁;

R₁₁ each independently is H or an amine protecting group; and

said functional group is selected from free amino, carboxyl or hydroxyl,

but excluding the monomer units wherein Z is a monomer of the formula IIm,

wherein R₁ is —(CH₂)₂—, and R₂ is —CH₂—; Y is —CR′₂—CO—; and X is (i) 2,6-diaminopurine-9-yl or an amino protected moiety thereof, i.e., the chemical moiety X₁, wherein R₄ each independently is H or an amine protecting group, and R₅ is H; (ii) 2-amino-6-oxopurine-9-yl or an amino protected moiety thereof, i.e., the chemical moiety X₅, wherein R₄ is H or an amine protecting group, R₅ is H, and R₆ is O or (iii) 4-amino-2-oxo-3-pyrimidinium-1-yl or an amino protected moiety thereof, i.e., the chemical moiety X₆, wherein R₄ is H or an amine protecting group, R₅ is H, and R₆ is O.

The term “amine protecting group”, as used herein, refers to any group that may be introduced into the monomer unit of the invention by chemical modification of an amine in order to obtain chemoselectivity in the subsequent polymerization of said monomer unit. Non-limiting examples of amine protecting groups include benzyloxycarbonyl (carbobenzyloxy, Cbz), 9-fluorenylmethyloxy carbonyl (Fmoc), p-methoxybenzyl carbonyl, tert-butyloxycarbonyl (Boc), 3,4-dimethoxybenzyl, p-methoxyphenyl, tosyl, N-phthalimide, N-2,5-dimethylpyrrole, benzyl and triphenylmethyl.

In certain embodiments, Y in the monomer unit of the invention is —CR′₂—CO— or —CR′₂—CS—, preferably —CR′₂—CO—, wherein R′ each independently is H or a (C₁-C₂)alkyl, preferably methyl, optionally substituted with at least one functional group selected from free amino, carboxyl or hydroxyl; and Z is a monomer of the formula IIm as defined above, preferably wherein R₁ is —(CH₂)₂— and R₂ is —CH₂—; or R₁ is —CH₂— and R₂ is —(CH₂)₂—. In particular embodiments, Y is —CH₂—CO—; and Z is a monomer of the formula IIm, wherein R₁ is —(CH₂)₂—, R₂ is —CH₂—, and R₁₁ is t-butoxycarbonyl (Boc).

In other certain embodiments, Y in the monomer unit of the invention is a covalent bond; and Z is a monomer of the formula IIIm or IVm. In particular embodiments, Z is a monomer of the formula IIIm, wherein R₃ is selected from —O⁻, —OH, —S⁻, —SH, or a (C₁-C₂)alkyl optionally substituted with at least one functional group. In other particular embodiments, Z is a monomer of the formula IVm, wherein R₃ is NR″₂ wherein R″ each independently is H or a (C₁-C₂)alkyl optionally substituted with at least one functional group.

The specific monomer units described in the specification are all based on a polyamide backbone (Z is a monomer of the formula IIm). These monomer units are herein identified as monomers M₁₋₁ₐ, M_{1-1b} (excluded from the definition of the general formula Im), M₁₋₂ₐ, M₁₋₃ₐ, M_{1-3b}, M₁₋₄ₐ, M_{1-4b}, M_{1-5b}, M₁₋₆ₐ, M₄₋₁ₐ, M_{4-1b}, M₄₋₂ₐ, M_{4-2b}, M_{4-3b}, M₄₋₄ₐ, M₅₋₁ₐ, M₆₋₁ₐ and M_{6-1b}, and their full chemical structures are depicted in Table 3 hereinafter.

In certain specific embodiments, the monomer unit of the invention is a compound of the general formula Im, wherein Y is —CH₂—CO—; Z is a monomer of the formula IIm, wherein R₁ is —(CH₂)₂—, R₂ is —CH₂—, and R₁₁ is t-butoxycarbonyl; and (i) X is a chemical moiety of the general formula X₁, wherein R₄ of the amine group linked to the carbon atom at position 2 of the purine moiety is R₁₁, wherein R₁₁ is H; R₄ of the amine group linked to the carbon atom at position 6 of the purine moiety is COR₉, wherein R₉ is methyl; and R₅ is H (monomer unit M₁₋₂ₐ); (ii) X is a chemical moiety of the general formula X₁, wherein R₄ of the amine group linked to the carbon atom at position 2 of the purine moiety is R₁₁, wherein R₁₁ is H; R₄ of the amine group linked to the carbon atom at position 6 of the purine moiety is COR₉, wherein R₉ is (CH₂)₂NHR₁₀, R₁₀ is R₁₁, and R₁₁ is H or Cbz; and R₅ is H (monomer units M₁₋₃ₐ and M_{1-3b}, respectively); (iii) X is a chemical moiety of the general formula X₁, wherein R₄ of the amine group linked to the carbon atom at position 2 of the purine moiety is COR₉, wherein R₉ is (CH₂)₂NHR₁₀, R₁₀ is R₁₁, and R₁₁ is H; R₄ of the amine group linked to the carbon atom at position 6 of the purine moiety is R₁₁, wherein R₁₁ is H or Cbz; and R₅ is H (monomer units M₁₋₄ₐ and M_{1-4b}, respectively); (iv) X is a chemical moiety of the general formula X₁, wherein R₄ of the amine group linked to the carbon atom at position 2 of the purine moiety is R₁₁, wherein R₁₁ is H; R₄ of the amine group linked to the carbon atom at position 6 of the purine moiety is R₉, wherein R₉ is —(CH₂)₃NHR₁₀, R₁₀ is R₁₁, and R₁₁ is Cbz; and R₅ is H (monomer unit M_{1-5b}); (v) X is a chemical moiety of the general formula X₁, wherein R₄ of the amine group linked to the carbon atom at position 2 of the purine moiety is R₁₁, wherein R₁₁ is H; R₄ of the amine group linked to the carbon atom at position 6 of the purine moiety is R₉, wherein R₉ is —(CH₂)₃N(R₁₀)₃⁺, and R₁₀ each is CH₃; and R₅ is H (monomer unit M₁₋₆ₐ); (vi) X is a chemical moiety of the general formula X₄, wherein R₄ is R₁₁, wherein R₁₁ is I or Cbz; R₅ is H; and R₆ is O (monomer units M₄₋₁ₐ and M_{4-1b}, respectively); (vii) X is a chemical moiety of the general formula X₄, wherein R₄ is COR₉, wherein R₉ is (CH₂)₂NHR₁₀, R₁₀ is R₁₁, and R₁₁ is H or Cbz; R₅ is H; and R₆ is O (monomer units M₄₋₂ₐ and M_{4-2b}, respectively); (viii) X is a chemical moiety of the general formula X₄, wherein R₁ is R₉, wherein R₉ is (CH₂)₃NHR₁₀, R₁₀ is R₁₁, and R₁₁ is Cbz; R₅ is H; and R₆ is O (monomer unit M_{4-3b}); (ix) X is a chemical moiety of the general formula X₄, wherein R₄ is R₉, wherein R₉ is (CH₂)₃N(R₁₀)₃⁺, and R₁₀ each is CH₃; R₅ is H; and R₆ is O (monomer unit M₄₋₄ₐ); (x) X is a chemical moiety of the general formula X₅, wherein R₄ is R₁₁, wherein R₁₁ is H; R₅ is H; and R₆ is O (monomer unit M₅₋₁ₐ); or (xi) X is a chemical moiety of the general formula X₆, wherein R₄ is R₁₁, wherein R₁₁ is H or Cbz; R₅ each is H; and R₆ is O (monomer units M₆₋₁ₐ and M_{6-1b}, respectively).

TABLE 3
Specific monomer units described in the specification
M₁₋₁ₐ 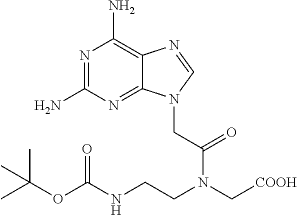 [SMILES: CC(C)(C)OC(=O)NCCN(CC(=O)O)C(=O)Cn1cnc2c(N)nc(N)nc21]
M_{1-1b}* 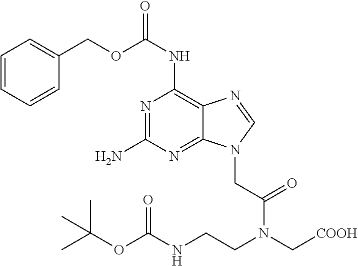 [SMILES: CC(C)(C)OC(=O)NCCN(CC(=O)O)C(=O)Cn1cnc2c(NC(=O)OCc3ccccc3)nc(N)nc21]
M₁₋₂ₐ 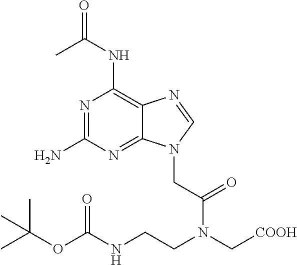 [SMILES: CC(=O)Nc1nc(N)nc2c1ncn2CC(=O)N(CCNC(=O)OC(C)(C)C)CC(=O)O]
M₁₋₃ₐ 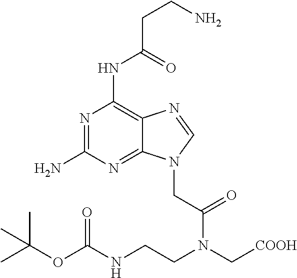 [SMILES: CC(C)(C)OC(=O)NCCN(CC(=O)O)C(=O)Cn1cnc2c(NC(=O)CCN)nc(N)nc21]
M_{1-3b}* 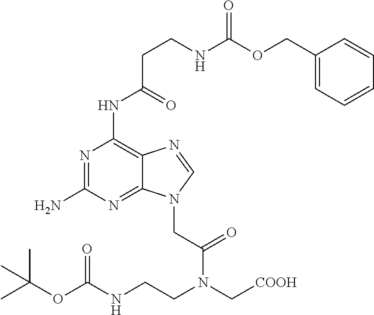 [SMILES: CC(C)(C)OC(=O)NCCN(CC(=O)O)C(=O)Cn1cnc2c(NC(=O)CCNC(=O)OCc3ccccc3)nc(N)nc21]
M₁₋₄ₐ 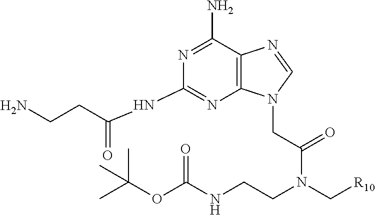
M_{1-4b}* 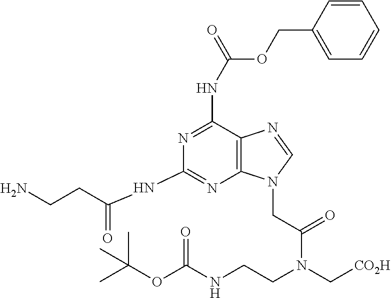 [SMILES: CC(C)(C)OC(=O)NCCN(CC(=O)O)C(=O)Cn1cnc2c(NC(=O)OCc3ccccc3)nc(NC(=O)CCN)nc21]
M_{1-5b}* 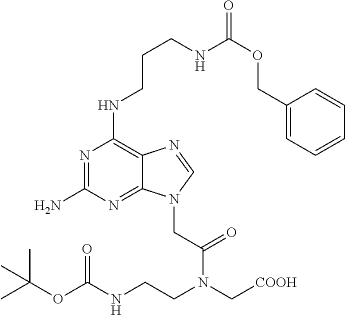 [SMILES: CC(C)(C)OC(=O)NCCN(CC(=O)O)C(=O)Cn1cnc2c(NCCCNC(=O)OCc3ccccc3)nc(N)nc21]
M₁₋₆ₐ 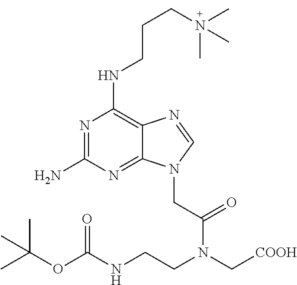
M₄₋₁ₐ 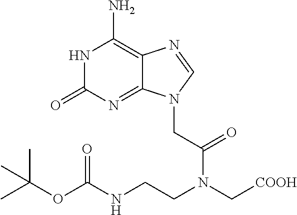 [SMILES: CC(C)(C)OC(=O)NCCN(CC(=O)O)C(=O)Cn1cnc2c(N)[nH]c(=O)nc21]
M_{4-1b}* 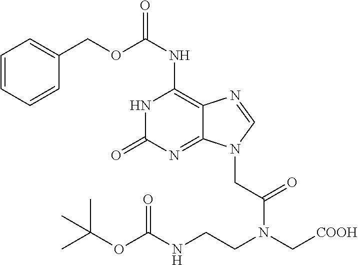 [SMILES: CC(C)(C)OC(=O)NCCN(CC(=O)O)C(=O)Cn1cnc2c(NC(=O)OCc3ccccc3)[nH]c(=O)nc21]
M₄₋₂ₐ 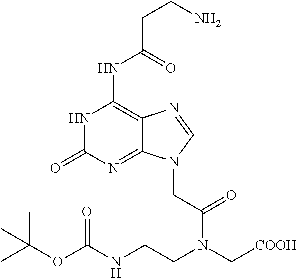 [SMILES: CC(C)(C)OC(=O)NCCN(CC(=O)O)C(=O)Cn1cnc2c(NC(=O)CCN)[nH]c(=O)nc21]
M_{4-2b}* 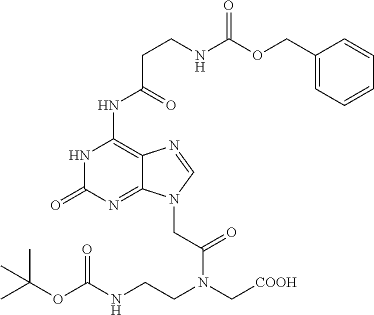 [SMILES: CC(C)(C)OC(=O)NCCN(CC(=O)O)C(=O)Cn1cnc2c(NC(=O)CCNC(=O)OCc3ccccc3)[nH]c(=O)nc21]
M_{4-3b}* 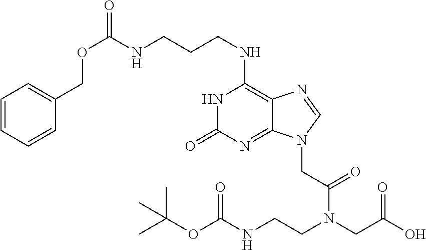 [SMILES: CC(C)(C)OC(=O)NCCN(CC(=O)O)C(=O)Cn1cnc2c(NCCCNC(=O)OCc3ccccc3)[nH]c(=O)nc21]
M₄₋₄ₐ 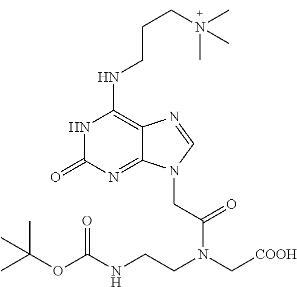
M₅₋₁ₐ 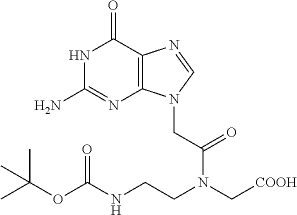 [SMILES: CC(C)(C)OC(=O)NCCN(CC(=O)O)C(=O)Cn1cnc2c(=O)[nH]c(N)nc21]
M₆₋₁ₐ 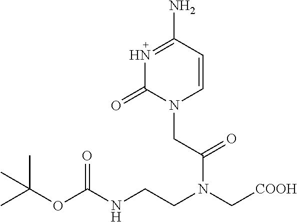 [SMILES: CC(C)(C)OC(=O)NCCN(CC(=O)O)C(=O)Cn1ccc(N)nc1=O]
M_{6-1b}* 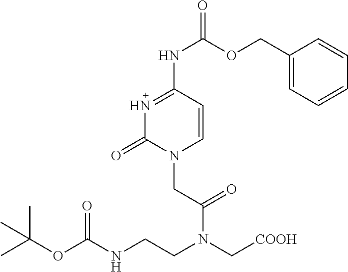 [SMILES: CC(C)(C)OC(=O)NCCN(CC(=O)O)C(=O)Cn1ccc(NC(=O)OCc2ccccc2)nc1=O]
*The Cbz group is an amine protecting group and could thus be replaced by any other amine protecting group as defined above

The monomer units of the present invention may be synthesized according to any technology or procedure known in the art, e.g., as described in Examples 3-12 and depicted in Schemes 6-17 hereinafter.

Example 13 describes a study in which the ability of two of the monomer units exemplified, in particular M₁₋₆ₐ and M₄₋₄ₐ, to effectively and selectively bind to DNA double-stranded sequences consisting of either A-T or C-G base pairs only was tested using the Real-Time PCR method. As shown, an analog of M₁₋₆ₐ, designed to be specific for A-T base pairs only, increased the T_{M} of 25 mer A-T-DNA from 56.1° C. in the control to 63° (in the treated well, whereas it did not affect the T_{M} of 25 mer C-G-DNA; and analog of M₄₋₄ₐ, designed to be specific for C-G base pairs only, increased the T_{M} of 25 mer C-G-DNA from 84.5° C. in the control to 90.1° C. in the treated well; whereas it did not affect the T_{M} of 25 mer A-T-DNA.

In order to prepare a triplex forming molecule according to the invention, the monomer units synthesized are then polymerized utilizing any suitable technique known in the art, e.g., solid phase technology as shown in Example 14. The number of monomer units in the triplex forming molecule prepared and their order are determined according to the target nucleic acid molecule to be treated, i.e., bonded, by the triplex forming molecule prepared.

Since initiating synthesis of polymers on solid phase using bulky monomer is problematic, the synthesis of the triplex forming molecules may start, as shown in Example 14, with glycine, beta-alanine, or any other un-branched amino acid so as to increase yield. As further shown, in order to enable further electrostatic interactions between the triplex forming molecule and the phosphoric groups of the target double-stranded DNA backbone, thus further increasing the energy required so as to dissociate the nucleic acid molecule into two separate strands, a chain of three of four 2-(2-aminoethylamino) acetic acid units, each positively charged under physiological conditions, was linked to the N- and/or C-terminus of the triplex forming molecule backbone.

As shown above and further demonstrated in the Examples section, the triplex forming molecules of the present invention are capable of specifically and efficiently interacting with double-stranded nucleic acid molecules thereby significantly decreasing dissociation of the double-stranded nucleic acid molecule to single strands. In sharp contrast to the triplex forming molecules currently known, the binding compounds of the invention interact with both strands of the nucleic acid molecule and are capable of recognizing all four base pairs of the DNA, as well as the additional base pairs of the RNA. By interacting with both strands of the nucleic acid molecule, the triplex forming molecule of the invention provides a “glue” to the double-stranded nucleic acid molecule, i.e., strengthens the interactions between the two strands, and therefore substantially increase the energy required so as to dissociate the nucleic acid molecule into two separate strands. Positive charges along the triplex forming molecule, i.e., as part of the pharmacophore of at least some of the chemical moieties X, could further increase the solubility of the triplex forming molecules and the cellular uptake and/or membrane penetration thereof. The membrane penetration of a triplex forming molecule of the invention may be studied, e.g., by linking a fluorescent unit to one of the triplex forming molecule termini. A non-limiting example of such a fluorescent unit, capable of linking via an amide bond to a triplex forming molecule based on a polyamide backbone, is 6-(7-nitrobenzo[c][1,2,5]oxadiazol-4-ylamino)hexanoic acid.

In view of the aforesaid, in certain embodiments, the binding compound of the present invention, as defined above, further comprises a chain of one or more units, each positively charged under physiological conditions, wherein said one or more units are linked one to each other via an amide bond, and said chain is linked via an amide bond to a linker that is further linked via an amide- or ester-bond to a terminal Z monomer of the polymeric structure. In certain particular embodiments, Z is a monomer of the formula II and said chain of one or more units is linked to a linker that is further linked via an amide bond to said terminal Z monomer; or Z is a monomer of the formula III or IV and said chain of one or more units is linked to a linker that is further linked via an ester bond to said terminal Z monomer. Non-limiting examples of units positively charged under physiological condition that can be used according to the present invention include 2-(2-aminoethylamino)acetic acid (herein identified P; see Scheme 19), as well as derivatives thereof in which the ethylene moiety and/or the methylene moiety is substituted with a (C₁-C₃)alkyl, —O—(C₁-C₃)alkyl, —OH, or halogen. The linkers that can be used according to the present invention are preferably about 10-15 Å long. Examples of such linkers include, without being limited to, 2-(2-(2-aminoethoxy)ethoxy)acetic acid (herein identified L1, see Scheme 19); 3-(4-aminobutanoyl-oxy)propanoic acid (herein identified L2, see Scheme 19); 7-aminoheptanoic acid, 9-aminononanoic acid, 1-aminoundecanoic acid, 13-aminotridecanoic acid, and 15-aminopentadecanoic acid (herein identified L3, wherein n is 2-6, respectively; see Scheme 19); and 8-aminooct-4-enoic acid, 10-aminodeca-3,6-dienoic acid, 14-aminotetradeca-4,7,10-trienoic acid, and 17-aminoheptadeca-4,7,10,13-tetraenoic acid (herein identified L4, wherein n is 1-4, respectively, see Scheme 19).

In a further aspect, the present invention thus provides a pharmaceutical composition comprising a sequence specific double-stranded DNA/RNA binding compound as defined above, or a pharmaceutically acceptable salt thereof, and a pharmaceutically acceptable carrier.

In particular embodiments, the pharmaceutical composition of the invention comprises a binding compound in which each one of X independently is a chemical moiety of a general formula selected from formulas X₁-X₁₃ as defined above; Y is —CR′₂—CO— or —CR′₂—CS—, preferably —CR′₂—CO—, wherein R′ each independently is H or a (C₁-C₂)alkyl, preferably methyl, optionally substituted with at least one functional group; and Z is a monomer of the formula II, preferably wherein R₁ is —(CH₂)₂— and R₂ is —CH₂—; or R₁ is —CH₂— and R₂ is —(CH₂)₂—. In more particular such embodiments, each one of X independently is: (i) a chemical moiety of the general formula X₁, wherein R₄ of the amine group linked to the carbon atom at position 2 of the purine moiety is H; R₄ of the amine group linked to the carbon atom at position 6 of the purine moiety is H, —COCH₃ or —CO(CH₂)NH₂; and R₅ is H; (ii) a chemical moiety of the general formula X₁, wherein R₄ of the amine group linked to the carbon atom at position 2 of the purine moiety is —CO(CH₂)₂NH₂; R₄ of the amine group linked to the carbon atom at position 6 of the purine moiety is H; and R₅ is H; (iii) a chemical moiety of the general formula X₁, wherein R₄ of the amine group linked to the carbon atom at position 2 of the purine moiety is H; R₄ of the amine group linked to the carbon atom at position 6 of the purine moiety is —(CH₂)₃NH₂ or —(CH₂)₃N(CH₃)₃; and R₅ is H; (iv) a chemical moiety of the general formula X₄, wherein R₄ is H, —CO(CH₂)₂NH₂, —(CH₂)₃NH₂ or —(CH₂)₃N(CH₃)₃⁺; R₅ is H; and R₆ is O; (v) a chemical moiety of the general formula X₅, wherein R₄ is H; R₅ is H; and R₆ is O; (vi) a chemical moiety of the general formula X₆, wherein R₄ is H; R₅ each is H; and R₆ is O; or (vii) a chemical moiety of the general formula X₇, wherein R₄ is H; R₅ each is H; and R₆ is O.

The binding compounds and pharmaceutical compositions of the present invention can be provided in a variety of formulations, e.g., in a pharmaceutically acceptable form and/or in a salt form, e.g., hydrates, as well as in a variety of dosages.

The pharmaceutical composition provided by the invention may be prepared by conventional techniques, e.g., as described in Remington: The Science and Practice of Pharmacy, 19^{th} Ed., 1995. The composition may be in solid, semisolid or liquid form and may further include pharmaceutically acceptable fillers, carriers or diluents, and other inert ingredients and excipients. Furthermore, the pharmaceutical composition can be designed for a slow release of the binding compound. The composition can be administered by any suitable route, which effectively transports the active compound, i.e., the triplex forming molecule of the invention, to the appropriate or desired site of action. Suitable administration routes include, e.g., intravenous, intraarterial, intramuscular, subcutaneous, transdermal and topical administration; inhalation; and nasal, oral, sublingual, nasogastric, nasoenteric, orogastric, rectal and intraperitoneal administration. The dosage will depend on the state of the patient, and will be determined as deemed appropriate by the practitioner.

Suitable pharmaceutically acceptable salts include acid addition salts such as, without being limited to, those formed with hydrochloric acid, fumaric acid, p-toluenesulfonic acid, maleic acid, succinic acid, acetic acid, citric acid, tartaric acid, carbonic acid, or phosphoric acid. Salts of amine groups may also comprise quaternary ammonium salts in which the amino nitrogen atom carries a suitable organic group such as an alkyl, alkenyl, alkynyl, or aralkyl moiety. Furthermore, where the compounds of the invention carry an acidic moiety, suitable pharmaceutically acceptable salts thereof may include metal salts such as alkali metal salts, e.g., sodium or potassium salts, and alkaline earth metal salts, e.g., calcium or magnesium salts.

The pharmaceutical compositions of the present invention may comprise the active agent, i.e., the triplex forming molecule of the invention, formulated for controlled release in microencapsulated dosage form, in which small droplets of the active agent are surrounded by a coating or a membrane to form particles in the range of a few micrometers to a few millimeters, or in controlled-release matrix.

Another contemplated formulation is depot systems, based on biodegradable polymers, wherein as the polymer degrades, the active ingredient is slowly released. The most common class of biodegradable polymers is the hydrolytically labile polyesters prepared from lactic acid, glycolic acid, or combinations of these two molecules. Polymers prepared from these individual monomers include poly(D,L-lactide) (PLA), poly(glycolide) (PGA), and the copolymer poly(D,L-lactide-co-glycolide) (PLG).

The triplex forming molecules and the pharmaceutical compositions of the present invention can be used for various therapeutic applications such as site-specific modulation of gene expression and targeting of DNA or RNA damage. More particularly, these triplex forming molecules can be used as a practical treatment for certain genetic diseases by increasing or decreasing expression of genes that are transcribed at low or high levels, respectively. While decreasing expression level of a certain gene may result from direct bonding to a target sequence of that gene or of a promoter thereof; increasing expression level of a certain gene may result, e.g., from bonding to a target sequence of a suppressor of said gene.

The triplex forming molecules of the invention may further be used so as to target DNA or RNA damage. More particularly, by linking certain drugs, e.g., an anti-cancer drug such as an anthracycline, the triplex forming molecules of the invention may effectively deliver said drug to the specific site of action in the cell, thus, significantly increasing the specificity of said drug. In a similar way, restriction enzymes can also be linked to the triplex forming molecules of the invention to thereby enable site-specific DNA or RNA cleavage.

The attachment of biological active agents, i.e., drugs, to the triplex forming molecules of the invention may be performed by linking said active agents to one or more of the functional groups of components Y and/or Z in the general formulas I and Im, if exist.

Since the triplex forming molecules of the present invention, under physiological conditions, may contain positively charged monomers and possibly also poly-cationic vectors (polyP), it is assumed that these charges will aid in membrane permeability. However, to further increase the transport of the triplex forming molecules across the cellular membrane, to thereby increase drug access to its specific targets, these binding compounds may further be coupled to well known cationic membrane translocating peptides (MTPs), which have been shown to have practical uses for protein transduction (transport) across the cellular membrane. After crossing the cellular membrane, it is expected that the binding compounds consisting of cationic oligonucleotide-like structure will penetrate the nucleus freely through the nuclear pore complex (NPC). It should be noted that although coupling a drug with a MTP is an easy technique, a possible degradation of the drug might take place while passing through the cytoplasm and thus passive targeting may not be successful. Increasing the concentration may be helpful. If the binding compound is not able to reach its specific target in the nucleus, alternative approaches such as active targeting may be adapted.

Active targeting of the triplex forming molecule delivery into the nucleus: It has been documented that co-administration of nuclear localisation signal (NLS) peptides had a positive effect on nuclear uptake. In view of that, NLS may be coupled to the binding compounds. Various lengths of NLS are available and could be coupled to the binding compounds by way of N3 adenine adducts. Coupling a short NLS peptide directly to a polymer is to couple a polypeptide or protein containing an NLS to the polymer, either by reversible interaction, streptavidin-biotin binding technology, or covalent coupling. This technology has been utilized for transporting genes into the nucleus and proved to be successful to a great extent as proven by gene expression. Plenty of such exciting developments are documented in the literature.

Another technique is anionic liposomes: Such liposomes will be also utilized to deliver the polymer to its target. Because of their similarity to cell membranes, liposomes, microscopic bubbles of fatty molecules (lipids) surrounding a watery interior, have long been viewed as promising biocompatible drug delivery systems. The advantage of liposomal drug delivery systems is their ability to entrap both water-soluble hydrophillic drugs inside their watery core and water-insoluble hydrophobic drugs within their membrane bilayers. The triplex forming molecules of the invention which are of water-soluble nature can be easily delivered into cells. Liposomes can form a biocompatible lipid-polymer complex and gets endocytosed by the cell plasma membrane. These liposomes have also the ability to protect the polymer from attack by DNases, leading to efficient endocytosis and delivery.

The triplex forming molecules and the pharmaceutical compositions of the present invention can further be used for certain diagnostic applications in vitro.

In still a further aspect, the present invention thus provides a method of altering DNA transcription in a cell comprising exposing a double-stranded DNA in said cell to a sequence specific double-stranded DNA/RNA binding compound as defined above, or a pharmaceutically acceptable salt thereof.

In yet a further aspect, the present invention provides a method of altering gene expression in an organism comprising administering to said organism a sequence specific double-stranded DNA/RNA binding compound as defined above, or a pharmaceutically acceptable salt thereof.

The invention will now be illustrated by the following non-limiting Examples.

### EXAMPLES

### Abbreviations

ACN, acetonitrile; AcOH, acetic acid; Boc, tert-butyloxycarbonyl; Boc-Aeg-OEt.HCl, ethyl N-(Boc-aminoethyl)glycinate; Cbz, carbobenzyloxy; DCC, dicyclohexylcarbodiimide; DCM, dichloromethane; DCU, 1,3-dicyclohexylurea; DIEA, diiesopropyl ethylamine; DMAP, 4-(dimethylamino) pyridine; DMF, N,N-dimethylformamide; DhbtOH, 3,4-dihydro-3-hydroxy-4-oxo-1,2,3-benzotriazine; HATU, O-(7-azabenzo-trizol-1-yl)-1,1,3,3,tetra-methyluronium hexafluorophosphate; HOBT, 1-hydroxybenzo-triazole; HPLC, high-performance liquid chromatography; NMM, N-methylmorpholine; NMP, n-methylmorpholine; RT, room temperature; TBTU, O-(benzo triazol-1-yl)-N,N,N′,N′-tetramethyluronium tetrafluoroborate; TFA, trifluoroacetic acid; TFMSA, trifluoromethanesulfonic acid; THF, tetrahydrofuran; TLC, thin layer chromatography.

### Materials and Methods

2-(2-(2-(benzyloxycarbonylamino)-6-oxo-1H-purin-9(6H)-yl)-N-(2-(tert-butoxy carbonylamino)ethyl)acetamido)acetic acid; 2-(2-(4-(benzyloxycarbonylamino)-2-oxo pyrimidin-1(2H)-yl)-N-(2-(tert-butoxycarbonylamino) ethyl)acetamido)acetic acid; 2-(2-(2-amino-6-(benzyloxycarbonylamino)-9H-purin-9-yl)-N-(2-(tert-butoxycarbonylamino) ethyl)acetamido)acetic acid; ethyl 2-(N-(2-(tert-butoxycarbonylamino)ethyl)-2-(2,6-diamino-9H-purin-9-yl)acetamido)acetate; ethyl 2-(2-(6-amino-2-oxo-1H-purin-9(2H)-yl)-N-(2-(tert-butoxycarbonylamino)ethyl)acetamido)acetate; and 2-(N-(2-(tert-butoxy carbonylamino)ethyl)-2-(5-methyl-2,4-dioxo-3,4-dihydropyrimidin-1(2H)-yl)acetamido) acetic acid were purchased from ASM Research Chemicals (Hannover, Germany).

Fragments of Double-Stranded DNA

The primers listed in Table 4 were purchased from Sigma Aldrich (Israel). Primers number 1 and 3 are complementary and annealed to form CG/GC double-stranded DNA fragment, and primers number 5 and 7 are complementary and annealed to form AT double-stranded DNA fragment. The DNA complementary oligomers were hybridized in a medium salt buffer containing 10 mM NaCl, 10 mM sodium phosphate and 0.1 mM EDTA, pH 7.0. The samples were heated to 90° C. for 5 min, slowly cooled to 20° C. and left at 4° (1 until used for T_{M} measurements with RT-PCR.

TABLE 4

| | | SEQ |
|---|---|---|
| | | ID |
| Number | Primer Sequence (5′-3′) | No. |
| 1 | GGGCCGGGCCGGGCCGGGCCGGGCC | 1 |
| 2 | CCGGGCCGGGCCGGGCCGGGCCGGG | 2 |
| 3 | GGCCCGGCCCGGCCCGGCCCGGCCC | 3 |
| | (complementary to 1) | |
| 4 | CCCGGCCCGGCCCGGCCCGGCCCGG | 4 |
| | (complementary to 2) | |
| 5 | GAAAAAAAAAAAAAAAAAAAAAAC | 5 |
| 6 | CAAAAAAAAAAAAAAAAAAAAAAG | 6 |
| 7 | GTTTTTTTTTTTTTTTTTTTTTTC | 7 |
| | (complementary to 5) | |
| 8 | CTTTTTTTTTTTTTTTTTTTTTTG | 8 |
| | (complementary to 6) | |

Formation of Duplex/Triplex DNA

Duplex DNA (dsDNA-1,2) sequences are formed by incubating a solution of single-strand DNA-1 and single-strand DNA-2 (1:1) at 90° C. for 5 min, and slowly cooling down to RT for 1-2 hours. Treated Duplex DNA (dsDNA-1,2 bonded to a triplex forming molecule of the invention) is formed by incubating a solution of triplex forming molecule (TFM) of the invention and dsDNA-1,2 (1:1) at 37° C. for 36 hours.

Protocol for T_{M} Measurements of Duplex DNA and Treated Duplex DNA UV Spectrophotometer.

The melting temperature (T_{M}), i.e., the temperature at which a DNA double helix dissociates into single strands, measurements of Duplex DNA or Treated Duplex DNA are carried out using quartz cuvettes with an optical path length of 1 cm on a Cray 300 UV/vis spectrophotometer interfaced with a computer for data collection and analysis. The temperature is increased from 30° C. to 98° C. at the rate of 1° C./min, and the T_{M} is determined by plotting the first derivative of the absorbance at 260 nm vs. temperature profile. T_{M} is defined as the temperature at which half the molecules are single-stranded.

Real Time (RT) PCR.

The T_{M} of Duplex DNA or Treated Duplex DNA can further be measured using the RT-PCT method, having a sensitivity that is significantly higher that that of the UV spectra method. According to this method, 5 μl of a selected double-stranded DNA is incubated with a 5 μl solution of a selected compound at RT for 5 min; 10 μl HRM/SYB (high resolution melting/syber green I) (X2) are then added; and the T_{M} is then measured. The run program for T_{M} measurement is as following: keep temperature of final solution at 25° C. for 5 min, raise temperature up to 99° C. with a rate of 0.1° C./sec, and then cool to 37° C.

HPLC Analysis and Mass Spectroscopy

HPLC analysis was performed on Accela High Speed LC system (Thermo Fisher Scientific Inc.), consisting of Accela Pump, Accela Autosampler and Accela PDA detector, under the following conditions: (i) temperature of HPLC column (20° C.); (ii) temperature of the sample tray (15° C.); (iii) flow (150 μl/min); and (iv) volume of injection (2 μl). Solvent A (water+0.05% AcOH); Solvent B (ACN:water (95:5)+0.05% AcOH).

| Time (min) | Solvent A (%) | Solvent B (%) |
|---|---|---|
| 0 | 20 | 80 |
| 4 | 20 | 80 |

Wash

| | | |
|---|---|---|
| 5 | 0 | 100 |
| 10 | 0 | 100 |

Equilibration

| | | |
|---|---|---|
| 11 | 20 | 80 |
| 13 | 20 | 80 |

HPLC separation was carried out using Phenomenex Gemini C18 column (2×30 mm, particle size 3 μm).

The Accela LC system was coupled with the LTQ Orbitrap Discovery hybrid FT mass spectrometer (Thermo Fisher Scientific Inc.) equipped with an electrospray ionization ion source. Mass spectrometer was operated in the positive ionization mode, ion source parameters were as follows: spray voltage 3.5 kV, capillary temperature 250° C., capillary voltage −35 V, source fragmentation was disabled, sheath gas rate (arb) 30, and auxiliary gas rate (arb) 10. Mass spectra were acquired in the m/z 150-2000 Da range.

The LC-MS system was controlled and data were analyzed using Xcalibur software (Thermo Fisher Scientific Inc.).

### Example 1

### Synthesis and Activity of Certain Demonstrative Compounds Having a Pharmacophore Capable of Interacting with the A-T Base Pair

In order to demonstrate the activity of the heterocyclic core-based chemical moieties being used in the designing of the triplex forming molecules of the invention, 3-amino-N-(6-aminopyridin-2-yl)propanamide, herein identified AH-11, was synthesized as shown in Scheme 5 (see Appendix). Unlike the heterocyclic core-based moieties of the invention, AH-11 cannot be linked to a linker and through which to a polymer chain, and therefore cannot be used in the triplex forming molecules of the invention. Nevertheless, this compound has a pharmacophore capable of interacting with the A-T and T-A base pairs, and can thus be used so as to simulate the selective activity of the heterocyclic core-based moieties used for the preparation of the triplex forming molecules of the invention.

¹H NMR (500 MHz, DMSO): δ 6.58-δ 7.86 (m, 3H, Aromatic), δ 3.61 (t, 2H, N—CH₂—CH₂), δ 2.92 (t, 2H, N—CH₂—CH₂).

The interactions between the two strands in double-stranded DNA or RNA are composed, in fact, of the interactions between adenine (A) and thymine (T) bases in DNA (or A with uracil in RNA), and the interactions between cytosine (C) and guanine (G) bases. The interactions in G-C/C-G base pairs, in which three hydrogen bonds are formed between the bases, are stronger than the interactions in A-T/T-A base pairs, in which two hydrogen bonds only are formed. Therefore, the DNA melting temperature (T_{M}), i.e. the temperature at which a DNA double helix dissociates into single strands, is correlated with the content of G-C/C-G base pairs in the sequence, wherein the higher percentage of G-C/C-G base pairs results in a higher T_{M}. In view of that, T_{M} is used as a measure of the content of C-G base pairs in double-stranded DNA, and the effect on the T_{M} of double-stranded DNA consisting of A-T base pairs or G-C/C-G base pairs only is the common used indicator for specificity and selectivity of molecules designed for specifically binding to either A-T or G-C base pairs.

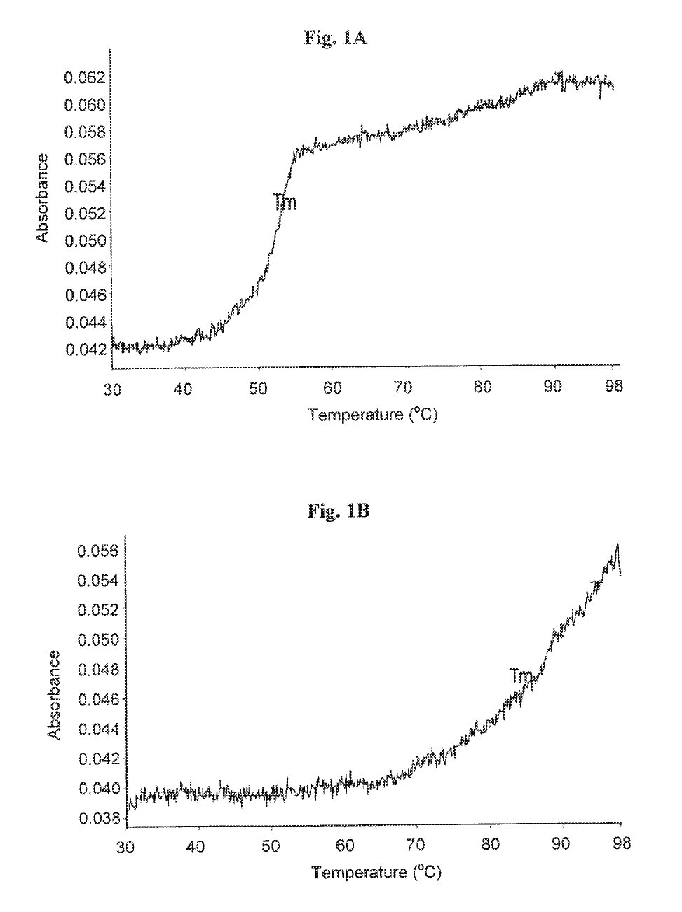

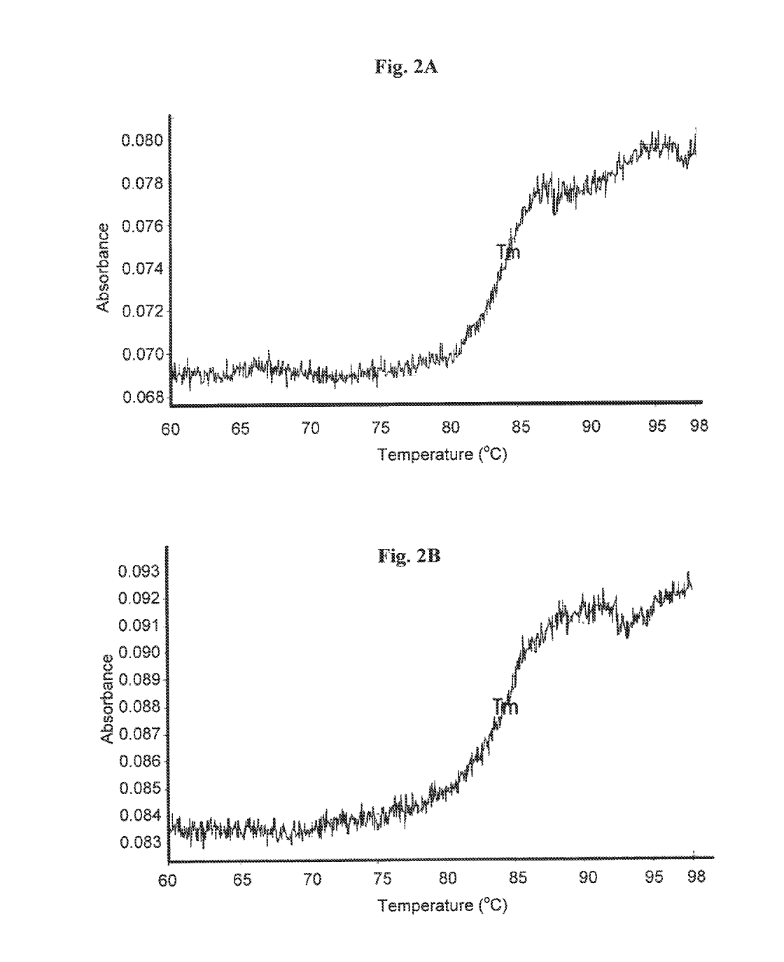

In order to test the effect of AH-11, designed to be specific for A-T base pair, on the T_{M} of DNA double-stranded sequences consisting of A-T or C-G base pairs only, two solutions each containing one of the aforesaid sequences were incubated with a solution containing the compound AH-11 at 37° C. for 36 hours, and treated double-stranded DNA were formed. The effect of AH-11 on the T_{M} of each one of the double-stranded DNA sequences was then tested by UV spectra at wavelength 260 nm as described in Materials and Methods. As found, AH-11 increased the T_{M} of 25 mer A-T-DNA from 53° C. to 85° (C, as shown in FIG. 1, whereas it did not affect at all the T_{M} of 25 mer C-G-DNA, as shown in FIG. 2.

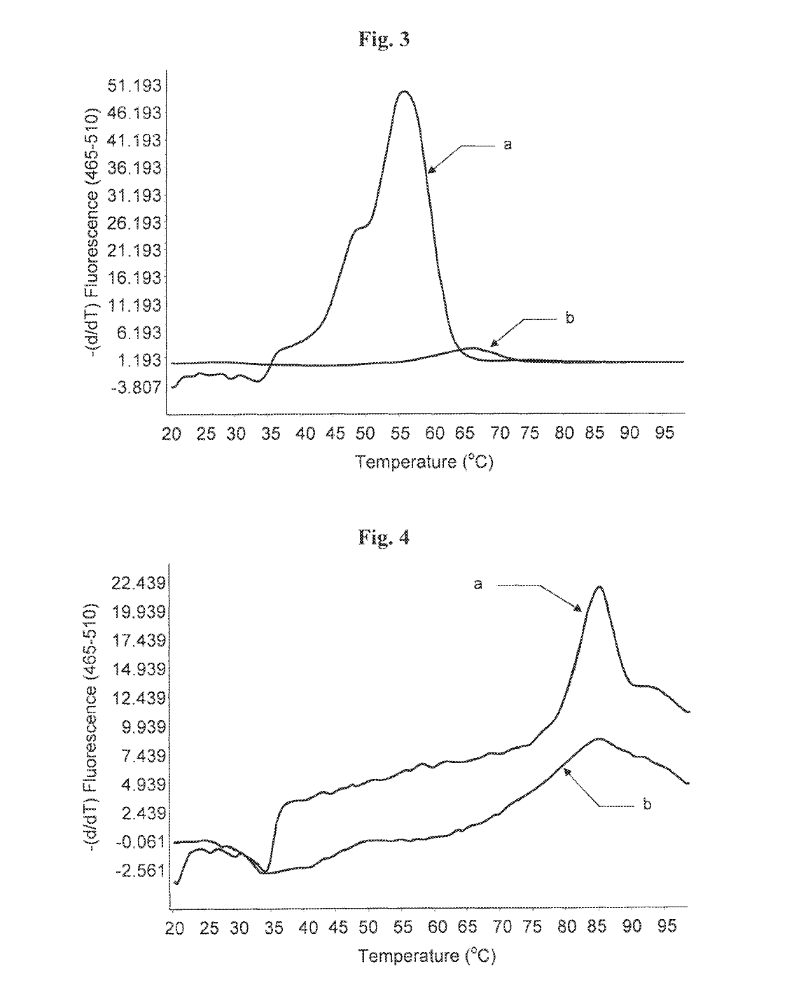

In a further study, the effect of AH-11 as well as three additional similar compounds having the same heterocyclic (pyridine) core on the T_{M} of DNA double-stranded sequences consisting of A-T or C-G base pairs only was tested by RT-PCR as described in Materials and Methods. The additional compounds tested are 4-amino-N-(6-aminopyridin-2-yl)butanamide, N,N′-(pyridine-2,6-diyl)bis(3-aminopropanamide), and N,N′-(pyridine-2,6-diyl)bis(4-aminobutanamide), herein identified AI-12, A1H-13 and AH-14, respectively (chemical structures are shown in Scheme 5). As found, the most effective compound among the four demonstrative compounds tested was AH-14, which increased the T_{M} of 25 mer A-T-DNA by 11.7° C. compared to the control (FIGS. 3-4). Compounds AH-11, AH-12 and AH-13 increased the T_{M} of 25 mer A-T-DNA by 4.2° C., 5.7° C. and 9.9° C., respectively. In all four cases, no effect was observed on the T_{M} of 25 mer C-G-DNA (the T_{M} shift range between 0.3 and 0.8° C.).

### Example 2

### Analyzing the Interactions Between Certain Moieties X and the Hoogsteen Face of the A-T or G-C Base Pair

In this study, the interactions between the pharmacophores of certain heterocyclic core-based moieties, in particular, chemical moieties X₁₋₁ and X₁₋₄, capable of interacting with the A-T base pairs, and X₄₋₂, X₅₋₁ and X₆₋₁, capable of interacting with the G-C base pairs, and the Hoogsteen face of A-T base pair or G-C base pair were analyzed using the ligand interactions application (MOE 2009.10. Chemical Computing Group). The ligand interactions application provides means to visualize an active site of a complex in diagrammatic form, wherein the diagram produced consists of the selected ligand as the centerpiece, which is drawn using the traditional schematic style for molecules. A selection of interacting entities, which includes hydrogen-bonded residues; close but non-bonded residues; solvent molecules; and ions, are drawn about the ligand and their positions in two-dimensions being chosen to be representative of the observed three-dimensions distances while further taking into account aesthetic considerations. Additional properties such as solvent accessible surface area and the ligand proximity outline are also shown.

The diagrams produced for each one of the moieties analyzed indicate the pharmacophore interactions of the moieties with either A-T base pair or G-C base pair, wherein: (i) the base ID numbers are prefixed by A or B (e.g. G B22, C A3, etc) to denote the parent strand; (ii) the gray filled black circles represent bases, e.g., A, T, C and G; (ii) the shaded area beyond the circumference of the black circles represent DNA contacts; (iii) the dotted arrows pointing to the bases represent hydrogen bond acceptors; and the dotted arrows pointing away from the bases represent hydrogen bond donors; (iv) the gray shaded spots represent ligand exposure to the solvent, wherein greater the spot the higher the exposure to the solvent; and (v) the dotted line represents a proximity contour, wherein the closer the proximity contour is to the pharmacophore, the lower its relatively spacious conditions.

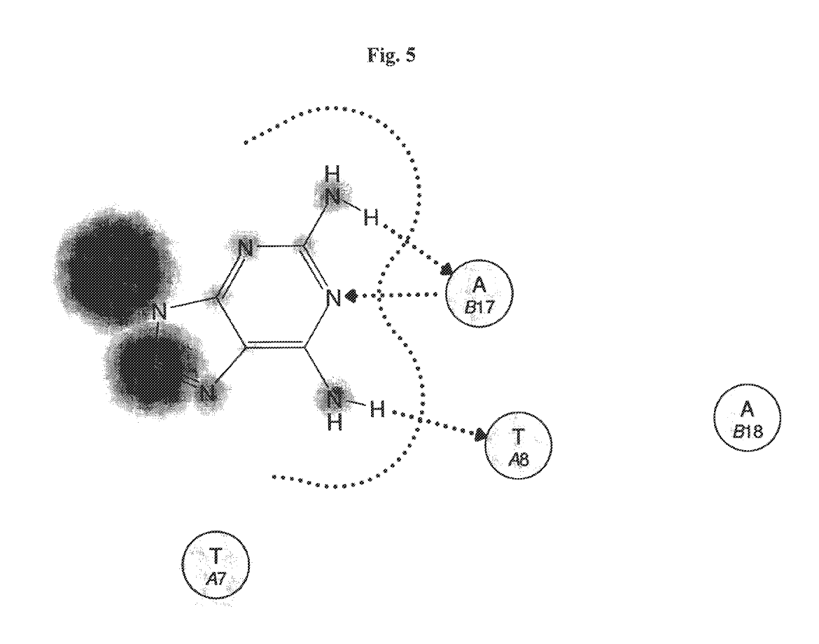

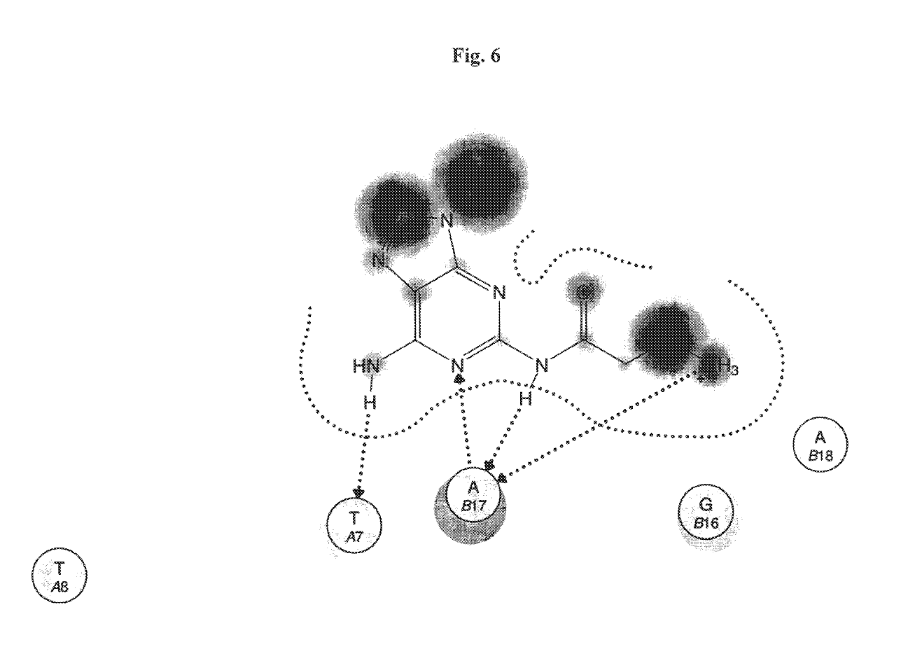

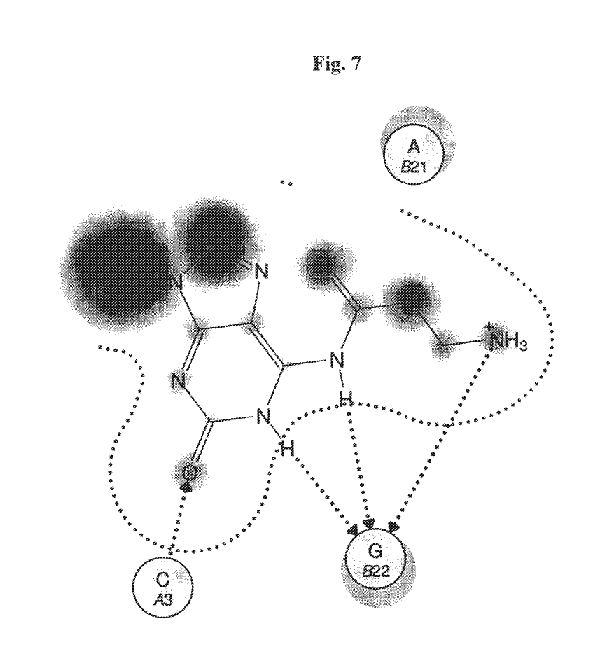

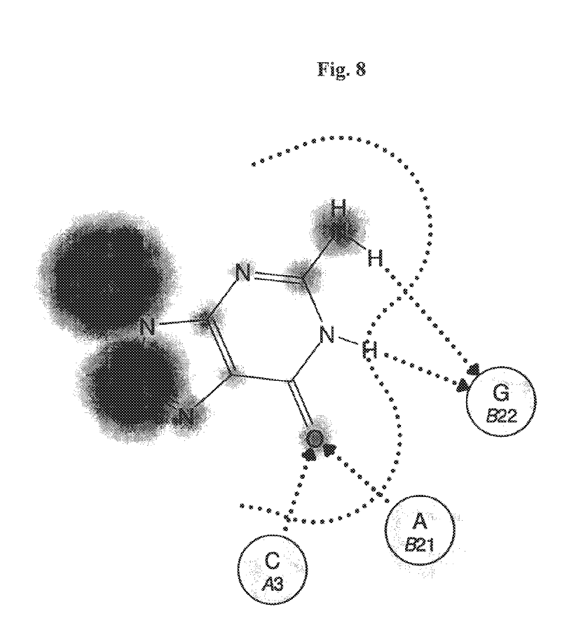

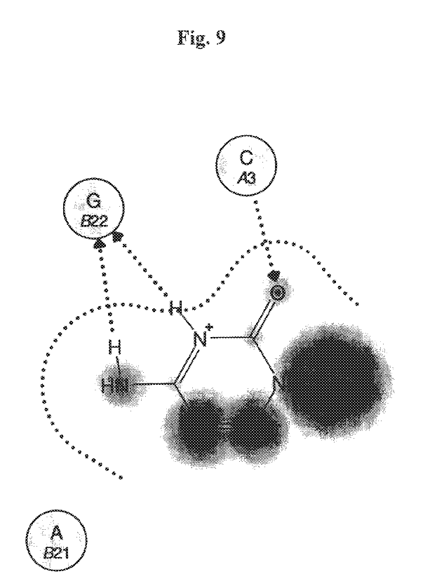

FIGS. 5-9 show that (i) the moiety X₁₋₁, having a pharmacophore representation of D2-A3-D4, forms hydrogen bond interactions with the A-T base pair, wherein the hydrogen bond acceptor (A3) and one of the hydrogen bond donors (D2 or D4) interact with the A base, and the other hydrogen bond donor (D2 or D4) interacts with the T base (FIG. 5); (ii) the moiety X₁₋₄, having a pharmacophore representation of D2-A3-D4-D5, forms hydrogen bonds and electrostatic interactions with A-T base pair, wherein the hydrogen bond acceptor (A3), one of the hydrogen bond donors (D4) and the positively charged moiety (D5) interact with the A base, and the other hydrogen bond donor (D2) interacts with the T base (FIG. 6); the moiety X₄₋₂, having a pharmacophore representation of A2-D3-D4-D5, forms hydrogen bonds and electrostatic interactions with G-C base pair, wherein the hydrogen bond acceptor (A2) interacts with the C base, and the hydrogen bond donors (D3 and D4) as well as the positively charged moiety (D5), interact with the G base (FIG. 7); and (iv) the moieties X₅₋₁ and X₆₋₁, each having a pharmacophore representation of A2-D3-D4, form hydrogen bond interactions with the G-C base pair, wherein the hydrogen bond acceptor (A2) interacts with the C base, and the hydrogen bond donors (D3 and D4) interact with the G base (FIGS. 8 and 9, respectively). As shown in all cases, the pharmacophore area in all the moieties demonstrated is more congested and less available to the solvent.

### Example 3

### Synthesis of 2-(2-(2-amino-6-(benzyloxy carbonylamino)-9H-purin-9-yl)-N-(2-(tert-butoxycarbonylamino)ethyl)acetamido)acetic acid, M_{1-1b}

2-(2-(2-amino-6-(benzyloxycarbonylamino)-9H-purin-9-yl)-N-(2-(tert-butoxy carbonylamino)ethyl)acetamido)acetic acid, M_{1-1b}, was synthesized as previously described (Komiyama M., Aiba Y., Ishizuka T., Sumaoka J., “Solid-phase synthesis of pseudo-complementary peptide nucleic acids”, Nature Protocols, 2008, 3, 646-654) and shown in Scheme 6 (see Appendix).

In particular, to the suspension of 26 g of 2,6-diaminopurine (173.17 mmol) in 500 ml DMF, sodium hydride (7.62 g, 189.84 mmol) was added, and the mixture was stirred for 3 h at RT under nitrogen. Ethyl bromoacetate (24.96 ml, 225.12 mmol) was then added and the mixture was stirred for another 2 h. In order to deactivate the excess sodium hydride, ethanol (60 ml) was added, the precipitate was filtrate on celite, and the filtrate was then evaporated by a rotary evaporator to half of the amounts of DMF. After the addition of ethanol (250 ml), the solution was kept at 0° C. over weekend, and a yellow precipitate was formed. Re-crystallization from ethanol yielded ethyl 2,6-diaminopurine-9-ylacetate as a crème product (31 g; 75%).

To the solution of ethyl 2,6-diaminopurine-9-ylacetate (8 g, 33.9 mmol) in 1,4 dioxane (250 ml), Rapoport's reagent (18.6 g, 50.8 mmol) was added at RT under nitrogen, and the mixture was stirred for 20 h. The solvent was then removed by a rotary evaporator, and the oily crude was purified by chromatography silica gel using CHCl₃/MeOH (3:1; Rf of compound b=0.74) to afford 2-amino-6-benzyloxycarbonylamino-purin-9-yl)-acetic acid ethyl ester as a solid (8.5 g, 68%).

2-amino-6-benzyloxycarbonylamino-purin-9-yl)-acetic acid ethyl ester (4 g) was dissolved in 50 ml of 2M NaOH, and after stirring at RT for 2 h. the aqueous solution was cooled to 0° C. and the product was precipitated by adjusting the pH to 2.5 with 2M HCl, yielding a white solid which was washed extensively with water. Drying under high vacuum gave (2-amino-6-benzyloxycarbonylamino-purin-9-yl)-acetic acid (3.4 g, 92%).

(2-amino-6-benzyloxycarbonylamino-purin-9-yl)-acetic acid (3 g, 8.76 mmol), as well as DCC (1.98 g, 9.66 mmol) and DhbtOH (1.57 g, 9.65 mmol), were added to DMF (50 ml) under nitrogen, and the mixture was stirred at RT for 1 h to obtain a homogeneous solution. Ethyl-N-(2-(t-butyloxycarbonylamino)ethyl)glycinate (2.37 g, 9.65 mmol) was then added, and the reaction was stirred overnight. DCU was removed by filtration and DMF was removed in vacuum. DCM (50 mil) was added and the organic layer was washed with saturated NaHCO₃, KHSO₄. After drying with Na₂SO₄, the solvent was removed to obtain an oily crude, which was purified by silica-gel chromatography using CH₃Cl₃/MeOH (10:1, Rf=60) to afford [[2-(2-amino-6-benzyloxycarbonylamino-purin-9-yl)-acetyl]-(2-tert-butoxycarbonyl amino-ethyl)-amino]-acetic acid (3.94 g, 79%).

[[2-(2-amino-6-benzyloxycarbonylamino-purin-9-yl)-acetyl]-(2-tert-butoxy carbonyl amino-ethyl)-amino]-acetic acid (3.3 g, 5.78 mmol) was stirred in 2M NaOH and THF (30 ml) for 2 h. The aqueous solution was cooled to 0° C., and the product was then precipitated by adjusting the pH to 4 with 2M HCl, yielding a white solid that was extensively washed with water. Drying under high vacuum gave [2-(2-(2-amino-6-(benzyloxycarbonylamino)-9H-purin-9-yl)-N-(2-(tert-butoxycarbonylamino)ethyl)acetamido)acetic acid], M_{1-1b}, (4.13 g, 76%).

### Synthesis of trifluoro-methanesulfonate 3-benzyloxycarbonyl-1-methyl-3H-imidazole-1-ium (Rapoport's Reagent)

Benzyl chloroformate was added to the suspension of imidazole in toluene at 0° C., and the mixture was stirred at RT overnight to form a white precipitate (imidazole hydrochloride). The precipitate was filtered, and the filtrate was evaporated to give N-benzyloxycarbonylimidazole as a crude oil, which was then purified by silica gel chromatography (1:1 hexane/ethyl acetate, R_{f} of compound=0.42).

N-benzyloxycarbonylimidazle (12 gram) was dissolved in DCM; methyltrifluoromethane (6.6 ml) was added drop wise to the solution at 0° C., and the mixture was stirred at RT for 30 minutes. Diethyl ether (30 ml) was added drop wise with stirring, and a precipitate was formed. The precipitate was filtrated and washed 3 times with ether (100 ml), and the final product was obtained as a white solid (18 g pure product, yielded 76%).

M_{1-1b} Characterization.

¹H NMR (500 MHz, DMSO): δ 7.81 (1H, s), 7.43 (5H, m), 6.33 (2H, bs), 5.17 (2H, s), 5.07 and 4.90 (2H, s), 4.30 and 3.99 (2H, s), 2.55 (2H, m), 1.38 (9H, s). This compound has 2 rotamers with respect to restricted rotation of the amide bond, so that corresponding NMR signals are observed in 7:3 ratio).

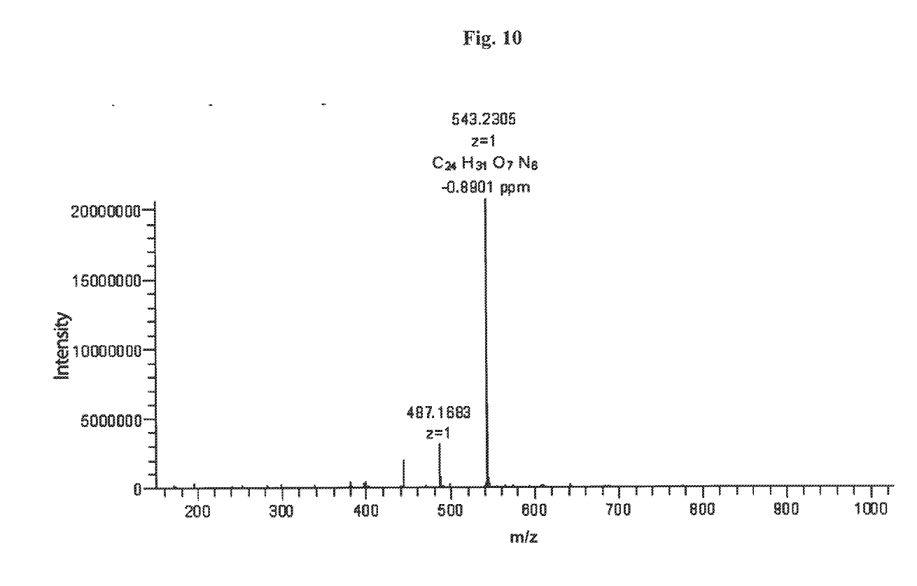

MS (ESI) (m/z) [M+H⁺]. Calculated for C₂₄H₃₁N₈O₇, 543.22. found 543.23 (FIG. 10).

### Example 4

### Synthesis of 2-(2-(6-acetamido-2-amino-9H-purin-9-yl)-N-(2-(tert-butoxy carbonylamino)ethyl)acetamido)acetic acid, M₁₋₂ₐ

2-(2-(6-acetamido-2-amino-9H-purin-9-yl)-N-(2-(tert-butoxycarbonylamino) ethyl)acetamido)acetic acid, M₁₋₂ₐ, is synthesized as shown in Scheme 7 (see Appendix).

The procedure for the synthesis of M₁₋₂ₐ is similar to that described in Example 3 for the synthesis of M_{1-1b}; however, in step 2, two methods are used for the coupling reaction between 3-benzyloxycarbonylamino-propionic acid and ethyl 2,6-diaminopurine-9-ylacetate, as follows:

### Reaction with Dicyclohexylcarbodiimide (DCC)

A solution of 3-benzyloxy carbonylamino-propionic acid (1 g, 4.48 mmol), HOBT (0.61 g, 4.48 mmol) and DCC (1.01 g, 4.93 mmol) in dry DMF (20 ml) was stirred at RT for 1 h, during which a white precipitate was formed. Ethyl 2,6-diaminopurine-9-ylacetate (1.06 g, 4.48 mmol) was added, and the reaction was then stirred for 24 h. DCU was removed by filtration and DMF was removed in vacuo. DCM (50 ml) was added and the organic layer was washed with saturated NaHCO₃, KHSO₄. After drying with Na₂SO₄ the solvent was removed to obtain an oily crude.

### Reaction with 2-ethoxy-1-ethoxycarbonyl-1,2-dihydroquinoline (EEDQ)

EEDQ (1.05 g, 4.23 mmol) was added to a stirred solution of 3-benzyloxycarbonylamino-propionic acid (1 g, 4.48 mmol) and ethyl 2,6-diaminopurine-9-ylacetate (1.06 g, 4.48 mmol) in THF (20 ml) and DMF (5 ml) at RT for 24 h, and the solvent was evaporated under reduced pressure. Water (10 ml) was added and the product was extracted twice with chloroform, washed with NaHCO₃ and brine, and dried over Na₂SO₄.

The products obtained in these two reactions were then used without further purification.

### Example 5

### Synthesis of 2-(2-(2-amino-6-(3-(benzyloxycarbonylamino)propan amido)-9H-purin-9-yl)-N-(2-(tert-butoxycarbonylamino)ethyl)acetamido)acetic acid, M_{1-3b}

2-(2-(2-amino-6-(3-(benzyloxycarbonylamino)propan amido)-9H-purin-9-yl)-N-(2-(tert-butoxycarbonylamino)ethyl)acetamido)acetic acid, M_{1-3b}, is synthesized as shown in Scheme 8 (see Appendix).

The procedure for the synthesis of M_{1-3b} is similar to that described in Example 3 for the synthesis of M_{1-1b}; however, in step 2, two methods are used for the coupling reaction between acetic acid and ethyl 2,6-diaminopurine-9-ylacetate, as follows:

### Reaction with dicyclohexylcarbodiimide (DCC)

A solution of acetic acid (1 g, 16.66 mmol), HOBT (2.25 g, 16.66 mmol) and DCC (3.78 g, 18.33 mmol) in dry DMF (20 nil) was stirred at RT for 1 h during which a white precipitate was formed. Ethyl 2,6-diaminopurine-9-ylacetate (1.06 g, 4.48 mmol) was added, and the reaction was stirred for 24 h. DCU was removed by filtration and DMF was removed in vacuo. DCM (50 mil) was added and the organic layer was washed with saturated NaHCO₃, KHSO₄. After drying with Na₂SO₄, the solvent was removed to obtain an oily crude.

### Reaction with 2-ethoxy-1-ethoxycarbonyl-1,2-dihydroquinoline (EEDQ)

EEDQ (4.1 g, 16.66 mmol) was added to a stirred solution of acetic acid (1 g, 16.66 mmol) and ethyl 2,6-diaminopurine-9-ylacetate (1.06 g, 4.48 mmol) in THF (20 ml) and DMF (5 ml), at RT for 24 h. The solvent was evaporated under reduced pressure. Water (10 ml) was added and the product was extracted twice with chloroform, washed with NaHCO₃ and brine, and dried over Na₂SO₄.

The products obtained in these two reactions were then used without further purification.

### Example 6

### Synthesis of 2-(2-(2-amino-6-(3-(benzyloxycarbonylamino)propyl amino)-9H-purin-9-yl)-N-(2-(tert-butoxycarbonylamino)ethyl)acetamido) acetic acid, M_{1-5b}

2-(2-(2-amino-6-(3-(benzyloxycarbonylamino)propylamino)-9H-purin-9-yl)-N-(2-(tert-butoxycarbonylamino)ethyl)acetamido)acetic acid, M_{1-5b}, is synthesized as shown in Scheme 9 (see Appendix).

In particular, to a solution of ethyl 2-(N-(2-(tert-butoxycarbonylamino)ethyl)-2-(2,6-diamino-9H-purin-9-yl)acetamido)acetate (1 mmol) and triethylamine (10 mmol) in dry DMF (10 ml) under nitrogen atmosphere is added dropwise (3-bromo-propyl)-carbamic acid benzyl ester (2 mmol) at RT. The reaction is stirred at 80° and monitored by TLC. After completion, the reaction solvent is evaporated to obtain ethyl 2-(2-(2-amino-6-(3-(benzyloxycarbonylamino)propylamino)-9H-purin-9-yl)-N-(2-(tert-butoxycarbonyl amino)ethyl)acetamido)acetate, which is purified by column chromatography. After stirring the purified compound with NaOH (0.5 M) for an hour, M_{1-5b} is obtained.

M_{1-5b} Characterization.

MS (ESI) (m/z) [M⁺]. Calculated for C₂₇H₃₈N₉O₇, 600.289.

### Example 7

### Synthesis of 3-(2-amino-9-(2-((2-(tert-butoxycarbonylamino)ethyl) (carboxymethyl)amino)-2-oxoethyl)-9H-purin-6-ylamino)-N,N,N-trimethyl propan-1-aminium, M₁₋₆ₐ

3-(2-amino-9-(2-((2-(tert-butoxycarbonylamino)ethyl)(carboxymethyl)amino)-2-oxoethyl)-9H-purin-6-ylamino)-N,N,N-trimethylpropan-1-aminium, M₁₋₆ₐ was synthesized as shown in Scheme 10 (see Appendix).

In particular, to a solution of ethyl 2-(N-(2-(tert-butoxycarbonylamino)ethyl)-2-(2,6-diamino-9H-purin-9-yl)acetamido)acetate (1 mmol) and triethylamine (10 mmol) in dry DMF (10 ml) under nitrogen atmosphere was added dropwise (3-bromo-propyl)-trimethyl-ammonium (2 mmol) at RT. The reaction was stirred at 80° and monitored by HPLC. After completion, the reaction solvent was evaporated to obtain 3-(2-amino-9-(2-((2-(tert-butoxycarbonylamino)ethyl)(2-ethoxy-2-oxoethyl)amino)-2-oxoethyl)-9H-purin-6-ylamino)-N,N,N-trimethyl propan-1-aminium, which was purified by column chromatography. After stirring the purified compound with NaOH (0.5 M) for an hour, M₁₋₆ₐ was obtained and was characterized by H¹NMR and Positive ESI MS.

M₁₋₆ₐ Characterization

MS (ESI) (m/z) [M⁺]. Calculated for C₂₂H₃₈N₉O₅, 508.299. found 508.29 (+Na, Exact mass 531.288. found 531.27).

### Example 8

### Synthesis of 2-(2-(6-(benzyloxycarbonyl amino)-2-oxo-1H-purin-9(2H)-yl)-N-(2-(tert-butoxycarbonylamino)ethyl)acetamido)acetic acid, M_{4-1b}

2-(2-(6-(benzyloxycarbonylamino)-2-oxo-1H-purin-9(2H)-yl)-N-(2-(tert-butoxy carbonylamino)ethyl)acetamido)acetic acid, M_{4-1b}, was synthesized as shown in Scheme 11 (see Appendix).

M_{4-1b} Characterization.

¹H NMR (500 MHz, DMSO): δ 7.4 (5H, m), 5.21 (2H, s), 4.25 (2H, s), 4.0 (2H, s), 3.50 (2H, m), 3.05 (2H, m), 1.34 (9H, s). This compound has 2 rotamers with respect to restricted rotation of the amide bond, so that corresponding NMR signals are observed in 7:3 ratio).

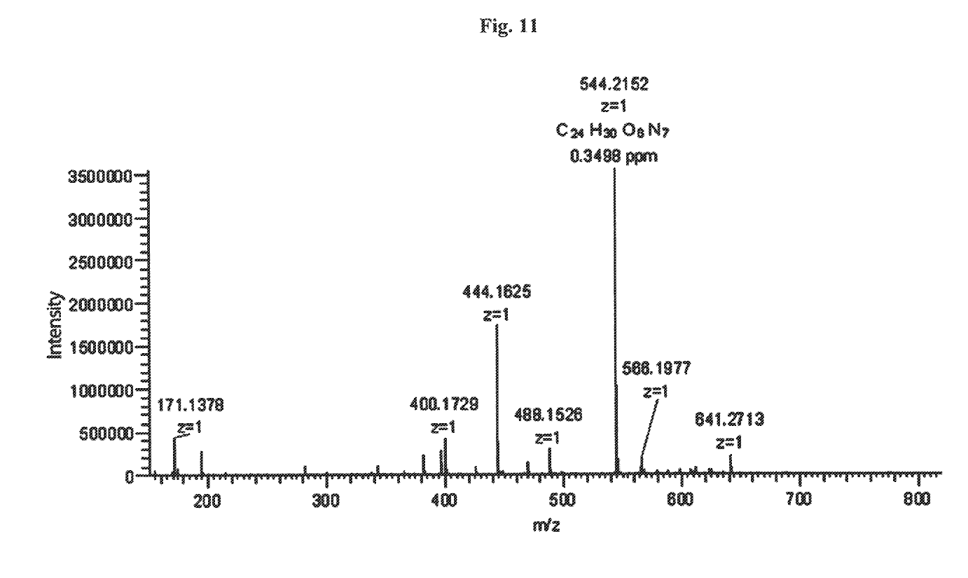

MS (ESI) (m/z) [M+H⁺]. Calculated for C₂₄H₃₀N₇O₈, 544.21. found 544.21 (FIG. 11).

An alternative procedure for the synthesis of compound M_{4-1b} is shown in Scheme 12 (see Appendix). This procedure is similar to that described in Example 3 for the synthesis of M_{1-1b}, starting from 6-amino-1,9-dihydro-purin-2-one instead of 9H-purine-2,6-diamine.

### Example 9

### Synthesis of 2-(2-(6-amino-2-oxo-1H-purin-9(2H)-yl)-N-(2-(tert-butoxy carbonylamino)ethyl)acetamido)acetic acid, M₄₋₂ₐ

2-(2-(6-amino-2-oxo-1H-purin-9(2H)-yl)-N-(2-(tert-butoxycarbonylamino)ethyl)acetamido)acetic acid, M₄₋₂ₐ, was synthesized as shown in Scheme 13 (see Appendix).

The ethyl 2-(2-(6-amino-2-oxo-1H-purin-9(2H)-yl)-N-(2-(tert-butoxycarbonyl amino)ethyl)acetamido)acetate obtained in step 6 of the synthesis described was characterized: ¹H-NMR (DMSO, 500 MHz): δ 7.61 (1H, s), 4.97 (2H, s), 4.1 (1H, m), 4.07 (1, m), 3.5 (2H, t), 2.0 (2H, s), 1.38 (9H, s), 1.26 (3H, m).

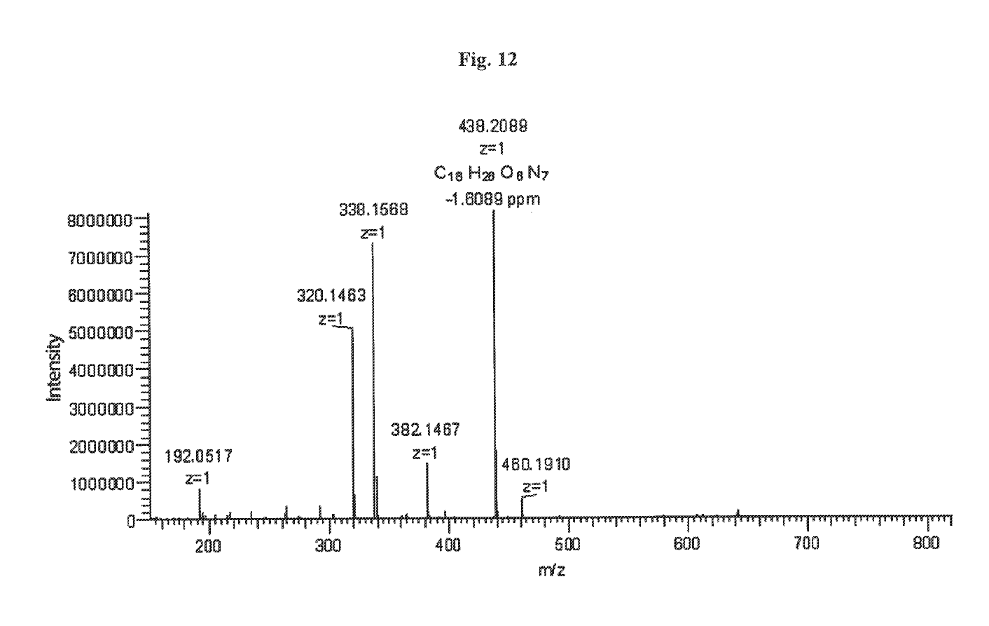

MS (ESI) (m/z) [M+H⁺]. Calculated for C₁₈H₂₈N₇O₆, 438.2. found 438.21 (FIG. 12).

### Example 10

### Synthesis of 2-(2-(6-(3-(benzyloxycarbonyl amino)propanamido)-2-oxo-1H-purin-9(2H)-yl)-N-(2-(tert-butoxycarbonyl amino)ethyl)acetamido) acetic acid, M_{4-2b}

2-(2-(6-(3-(benzyloxycarbonylamino)propanamido)-2-oxo-1H-purin-9(2H)-yl)-N-(2-(tert-butoxycarbonylamino)ethyl)acetamido)acetic acid, M_{4-2b}, is synthesized as shown in Scheme 14 (see Appendix), starting from ethyl 2-(2-(6-amino-2-oxo-1H-purin-9(2H)-yl)-N-(2-(tert-butoxycarbonylamino)ethyl)acetamido)acetate, obtained in step 6 of the synthesis shown in Scheme 13.

A solution of 3-benzyloxycarbonylamino-propionic acid (0.5 g, 2.24 mmol), DMAP and DCC (0.5 g, 2.46 mmol) in dry DMF was stirred under nitrogen, and the resulting mixture was stirred at RT for 1 h. Ethyl 2-(2-(6-amino-2-oxo-1H-purin-9(2H)-yl)-N-(2-(tert-butoxycarbonylamino)ethyl)acetamido) acetate (1 g, 2.46 mmol) was then added, and the reaction was stirred overnight. DCU was removed by filtration and DMF was removed in vacuum. DCM (50 ml) was added and the organic layer was washed with saturated NaHCO₃, KHSO₄. After drying over Na₂SO₄, the solvent was removed and cream foam was obtained. Re-crystallization with ETOH/H₂O gave a white solid powder (380 mg, 24%).

A solution of ethyl 2-(2-(6-(3-(benzyloxycarbonylamino)propanamido)-2-oxo-1H-purin-9(2H)-yl)-N-(2-(tert-butoxycarbonylamino)ethyl)acetamido)acetate (0.38 g, 1.7 mmol) in 2M NaOH (10 ml) and THF (2 ml) was stirred for 2 h. The aqueous solution was cooled to 0° C., and the product was precipitated by adjusting the pH to 4 with 4M HCl, yielding a white solid, which was washed extensively with water. Drying under high vacuum gave 2-(2-(6-(3-(benzyloxycarbonyl amino)propanamido)-2-oxo-1H-purin-9(2H)-yl)-N-(2-(tert-butoxycarbonylamino) ethyl)acetamido)acetic acid, M_{4-2b.}

### Preparation of 3-benzyloxycarbonylamino-propionic acid

Beta-alanine (0.113 mol, 10 g) was taken in a round bottom flask. Freshly distilled chlorotrimethylsilane (0.225 mol, 28.5 ml) was added slowly and stirred with a magnetic stirrer. Ethanol (100) ml) was then added and the resulting suspension was stirred at RT for 24 h. After the completion of reaction, as monitored by TLC, the reaction mixture was concentrated on a rotary evaporator to give beta-alanine ethyl ester hydrochloride as a white solid (15 g, yielded 86.5%).

The beta-alanine ethyl ester hydrochloride (15 g, 0.097 mol) was dissolved in DCM, triethylamine (30 ml) was added, and a precipitate was formed immediately and filtered. Benzylchloroformate (18 ml) was then added to the filtrate, and the reaction was stirred at RT for 2 h. Water (30 ml) was added, the solution was stirred for 5 min and extracted twice with DCM (100 nil), and the organic layer was washed with 5% NaOH and brine. After drying over Na₂SO₄, the solvent was removed by vacuum and the product (20 g, 81.2%) was obtained as colorless oil and was used without further purifications.

The starting material (15 g, 59.7 mmol) was dissolved in 2M NaOH (30 ml) and stirred for 1.5 h, and the solution was then acidified to pH=2.5 with HCl 5%. 3-benzyloxycarbonylamino-propionic acid (11.06 g, 82%) was formed as a white solid.

M_{4-2b} Characterization.

¹H-NMR (DMSO, 500 MHz): δ 7.84 (1H, m), 7.43 (5H, m), 5.21 (2H, s), 5.07 (1H, s), 4.88 (1H, s), 4.26 (1H, s), 3.98 (1H, s), 3.4 (2H, t), 3.29 (2H, t), 3.21 (2H, m), 1.3 (9H, s).

An alternative procedure for the synthesis of compound M_{4-2b} is shown in Scheme (see Appendix). This procedure is similar to that described in Example 5 for the synthesis of M_{1-3b}; however, the starting material in this case is 6-amino-1,9-dihydro-purin-2-one instead of 9H-purine-2,6-diamine.

### Example 11

### Synthesis of 2-(2-(6-(3-(benzyloxycarbonylamino)propylamino)-2-oxo-1H-purin-9(2H)-yl)-N-(2-(tert-butoxycarbonylamino)ethyl)acetamido) acetic acid, M_{4-3b}

2-(2-(6-(3-(benzyloxycarbonylamino)propylamino)-2-oxo-1H-purin-9(2H)-yl)-N-(2-(tert-butoxycarbonylamino)ethyl)acetamido) acetic acid, M_{4-3b}, was synthesized as shown in Scheme 16 (see Appendix).

In particular, to a solution of ethyl 2-(2-(6-amino-2-oxo-1H-purin-9(2H)-yl)-N-(2-(tert-butoxycarbonylamino)ethyl)acetamido)acetate (1 mmol) and triethylamine (10 mmol) in dry DMF (10 ml) under nitrogen atmosphere was added dropwise (3-bromo-propyl)-carbamic acid benzyl ester (2 mmol) at RT. The reaction was stirred at 80° and monitored by TLC. After completion, the reaction solvent was evaporated to obtain ethyl 2-(2-(6-(3-(benzyloxycarbonylamino)propylamino)-2-oxo-1H-purin-9(2H)-yl)-N-(2-(tert-butoxycarbonylamino)ethyl)acetamido)acetate, which was purified by column chromatography. After stirring the purified compound with NaOH (0.5 M) for an hour, M_{4-3b} was obtained and was characterized by H¹NMR and Positive ESI MS.

M_{4-3b} Characterization.

MS (ESI) (m/z) [M⁺]. Calculated for C₂₇H₃₇N₈O₈, 601.273. found 601.29.

### Example 12

### Synthesis of 3-(9-(2-((2-(tert-butoxycarbonylamino)ethyl)(carboxy methyl)amino)-2-oxoethyl)-2-oxo-2,9-dihydro-1H-purin-6-ylamino)-N,N,N-trimethylpropan-1-aminium, M₄₋₄ₐ

3-(9-(2-((2-(tert-butoxycarbonylamino)ethyl)(carboxymethyl)amino)-2-oxoethyl)-2-oxo-2,9-dihydro-1H-purin-6-ylamino)-N,N,N-trimethylpropan-1-aminium, M₄₋₄ₐ, was synthesized as shown in Scheme 17 (see Appendix).

In particular, to a solution of ethyl 2-(2-(6-amino-2-oxo-1H-purin-9(2H)-yl)-N-(2-(tert-butoxycarbonylamino)ethyl)acetamido)acetate (1 mmol) and triethylamine (10 mmol) in dry DMF (10 ml) under nitrogen atmosphere was added dropwise (3-bromo-propyl)-trimethyl-ammonium (2 mmol) at RT. The reaction was stirred at 80° and monitored by HPLC. After completion, the reaction solvent was evaporated to obtain 3-(9-(2-((2-(tert-butoxycarbonyl amino)ethyl)-2-ethoxy-2-oxoethyl)amino)-2-oxoethyl)-2-oxo-2,9-dihydro-1H-purin-6-ylamino)-N,N,N-trimethylpropan-1-aminium, which was purified by column chromatography. After stirring the purified compound with NaOH (0.5 M) for an hour, M₄₋₄ₐ was obtained and was characterized by H¹NMR and Positive ESI MS.

M₄₋₄ₐ Characterization.

MS (ESI) (m/z) [M⁺]. Calculated for C₂₂H₃₇N₈O₆, 509.283. found 509.40 (+Na, Exact mass 531.27. found 531.37) and (+K, Exact mass 547.24. found 547.19).

### Example 13

### The Effect of M₁₋₆ₐ and M₄₋₄ₐ on the T_{M} of DNA Double-Stranded Sequences Consisting of A-T or C-G Base Pairs Only

In this study, the ability of monomer units M₁₋₆ₐ, designed to be specific for A-T base pairs only, and M₄₋₄ₐ, designed to be specific for C-G base pairs only, to effectively and selectively bind to DNA double-stranded sequences consisting of A-T or C-G base pairs only was tested using the RT-PCR method as described in Material and Methods. Nevertheless, in order to neutralize a possible influence of the carboxylic groups of these monomers on the specificity and selectivity of the monomers, the compounds practically used were 3-(2-amino-9-(2-((2-(tert-butoxycarbonylamino)ethyl)(2-ethoxy-2-oxoethyl)amino)-2-oxoethyl)-9H-purin-6-ylamino)-N,N,N-trimethylpropan-1-aminium and 3-(9-(2-((2-(tert-butoxycarbonylamino)ethyl)(2-ethoxy-2-oxoethyl)amino)-2-oxoethyl)-2-oxo-2,9-dihydro-1H-purin-6-ylamino)-N,N,N-trimethylpropan-1-aminium, which are analogs of these monomer units M₁₋₆ₐ and M₄₋₄ₐ, respectively, in which the carboxyl groups are protected with ethyl (see Scheme 10 and 17).

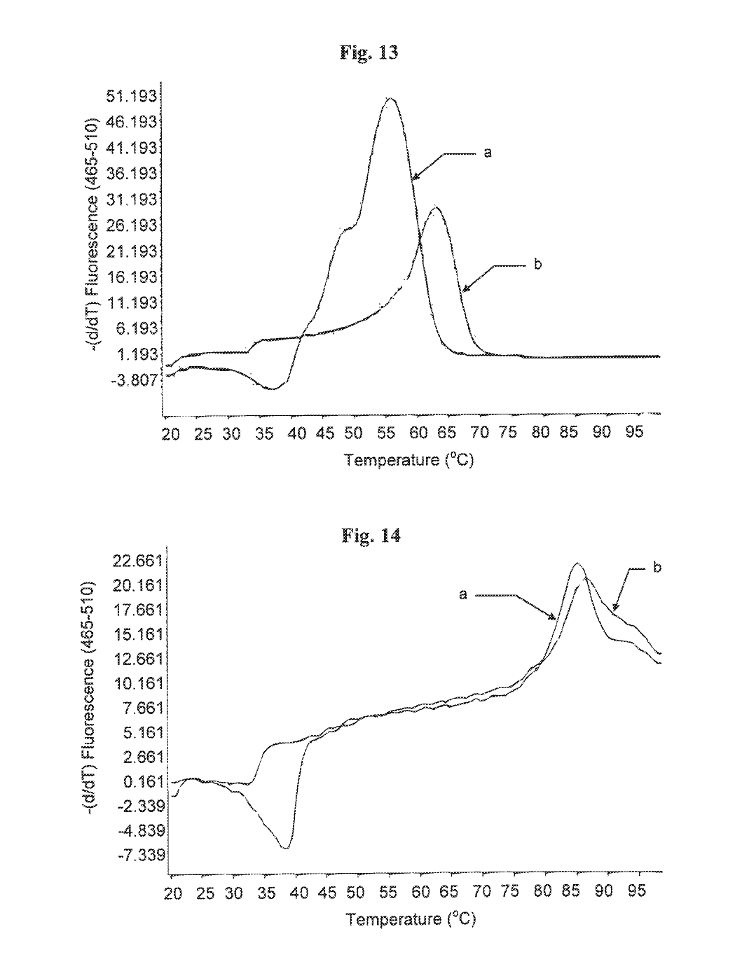

As found, M₁₋₆ₐ increased the T_{M} of 25 mer A-T-DNA from 56.1° C. in the control to 63° C. in the treated well; however, did not affect at all the T_{M} of 25 mer C-G-DNA, which was increased from 84.3° C. in the control well to 85.1° C. in the treated well (FIGS. 13-14). No difference was seen in the T_{M} of the treated 25 mer A-T-DNA well upon increasing the incubation time of said double-stranded DNA with a solution of said monomer unit from 5 min to 60 or 100 min.

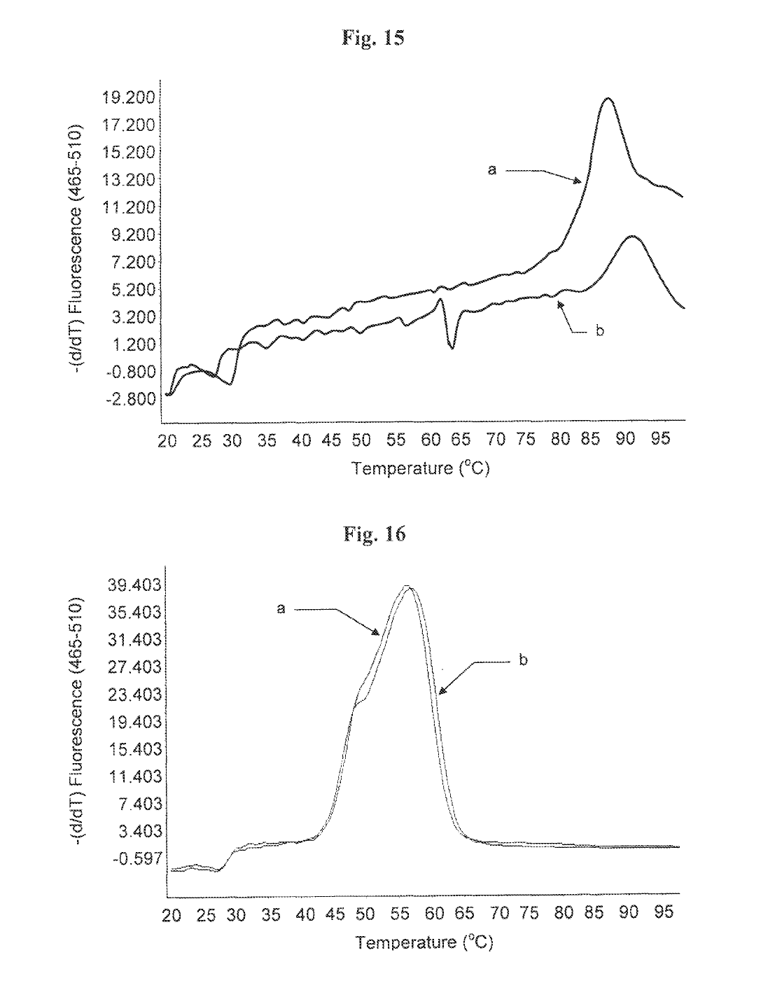

As further found, M₄₋₄ₐ increased the T_{M} of 25 mer C-G-DNA from 84.5° C. in the control to 90.1° C. in the treated well; however, did not affect at all the T_{M} of 25 mer A-T-DNA, which was increased from 57.1° C. in the control well to 57.4° (in the treated well (FIGS. 15-16).

### Example 14

### Synthesis of Sequence Specific Double-Stranded DNA Binding Compounds and Evaluation of their Biological Activity

(i) a polymer comprising 2-(N-(2-aminoethyl)-2-(2,6-diamino-9H-purin-9-yl)acetamido)acetic acid monomer, M₁₋₁ₐ, monomers

A demonstrative peptide nucleic acid (PNA) polymer composed of 7 bases of 2-(N-(2-aminoethyl)-2-(2,6-diamino-9H-purin-9-yl)acetamido)acetic acid monomer, M₁₋₁ₐ (7D, the sequence is presented from the N-terminus to the C-terminus, see Scheme 18. see Appendix) was synthesized as previously described (Komiyama M., Aiba Y., Ishizuka T., Sumaoka J. “Solid-phase synthesis of pseudo-complementary peptide nucleic acids”, Nature Protocols, 2008, 3, 646-654).

C₇₇H₁₀₁O₁₅N₅₆⁺. Molecular weight: 2049.89.

MS (ESI) (m/z) [M+H⁺]. Calculated for C₇₇H₁₀₁N₅₆O₁₅, 2049.89. found 2049.89.

### (ii) Additional PNA Polymers

The PNA polymers listed in Table 5 below were synthesized, using the monomer units herein identified M₁₋₁ₐ, M₄₋₁ₐ, M₄₋₄ₐ, M₅₋₁ₐ and M₆₋₁ₐ as building blocks. In two of the polymers synthesized, herein identified sequences C and L14, a fluorescent unit herein identified F, in particular, 6-(7-nitrobenzo[c][1,2,5]oxadiazol-4-ylamino)hexanoic acid (see Scheme 18) was linked via an amide bond to the N-terminus of the polymer. In order to enable electronic interactions between the polymer and the phosphoric groups of the target DNA backbone, thus increasing the ability of the polymer to efficiently interact with the target DNA sequence, a chain of 3-4 units each positively charged under physiological conditions (2-(2-aminoethylamino)acetic acid, P, see Scheme 19), was linked via a linker (2-(2-(2-aminoethoxy)ethoxy)acetic acid, L1, see Scheme 19) to the N-terminus, C-terminus, or both the N- and C-termini of the polymer synthesized.

TABLE 5
Polymers synthesized

| Name | Sequence* |
|---|---|
| Sequence B | P-P-P-L-M₅₋₁ₐ-M₅₋₁ₐ-M₆₋₁ₐ-M₆₋₁ₐ-M₆₋₁ₐ |
| Sequence C | P-P-P-L-M₁₋₁ₐ-M₅₋₁ₐ-M₁₋₁ₐ-M₄₋₁ₐ-M₅₋₁ₐ-M₁₋₁ₐ-M₄₋₁ₐ-M₅₋₁ₐ-F |
| Sequence E | B-M₄₋₄ₐ-M₄₋₄ₐ-M₅₋₁ₐ-M₅₋₁ₐ-M₅₋₁ₐ |
| Sequence H | B-M₄₋₁ₐ-M₆₋₁ₐ-T-M₅₋₁ₐ-M₆₋₁ₐ-T-M₅₋₁ₐ-M₆₋₁ₐ |
| Sequence H13 | B-M₄₋₁ₐ-M₆₋₁ₐ-T-M₅₋₁ₐ-M₆₋₁ₐ-T-M₅₋₁ₐ-M₆₋₁ₐ-L-P-P-P |
| Sequence L14 | P-P-P-P-L-M₄₋₁ₐ-M₅₋₁ₐ-M₁₋₁ₐ-M₄₋₁ₐ-M₅₋₁ₐ-M₁₋₁ₐ-M₄₋₁ₐ- |
| | M₁₋₁ₐ-F |

*T represents 2-(N-(2-(tert-butoxycarbonylamino) ethyl)-2-(5-methyl-2,4-dioxo-3,4-dihydropyrimidin-1(2H)-yl)acetamido)acetic acid; and B represents beta-alanine, linked to the solid phase prior to polymerization of the polymer due to technical considerations (see Scheme 19).

The polymers listed in Table 5 were synthesized using a solid phase technology previously described (Komiyama et al., 2008). For example, the polymer of sequence B was synthesized according to the following steps:

(i) Pretreatment of the Resin for Pseudo-Complementary (pc)PNA Synthesis.

This step was accomplished by DCM. For 0.01 mmole-50 mg of 4-methylbenzhydrylamine resin HCl salt was transfer to a Libra tube (reactor) and swell in 3.5 ml of DCM for 4 hours. The DCM was then removed, and the resin was washed with 5% DIEA/DCM for 3 min (three times).

(ii) Introduction of the First Residue: Z—N—(N-beta-Boc-aminoethyl)-Gly-OH(P).

0.015 mole of the chosen Boc-protected monomer, 0.015 mole of HATU and 0.03 mole of NMP was mixed with DMF solution, and incubated at RT for 2 h. The resin was washed with 50% DMF/DCM (2 ml), 5% DIEA/DCM (2 ml) and finally with dry DCM (2 ml). The washes were done twice.

(iii) Acetylation Reaction (Capping).

1 ml of a 1:2:2 acetic anhydride/DMF/pyridine solutions were added to the resin obtained in step (ii) and incubated at RT for 30 min. This step was repeated twice to complete the capping of the free amino groups. Washes with DMF (2 ml), 10% piperidine in DMF (2 ml) and finally with DCM twice. It should be noted that acetylation must be complete, otherwise the presence of free reactive amino groups will lead to the formation of undesired byproducts in the following steps.

(iv) Elongation of pcPNA (Deprotection Reaction).

1.5 ml of a 95:5 TFA/meta-cresol solution was added to the resin and incubated at RT for 15 minutes with vortexing. This procedure was repeated twice. The resin was then washed with 50% DMF/DCM (1 ml), 5% DIEA/DCM (1 ml) and finally with DCM (1 ml). The washes were repeated three times.

(v) Introduction of the Second Residue: Z—N—(N-beta-Boc-aminoethyl)-Gly-OH(P), (Coupling Reaction 2).

The coupling of step (i) was repeated; however, the amount of the moles was doubled to 0.03 mole of the PNA, HATU and 0.06 mole of NMP. The incubation time of this coupling reaction was 1-1.5 h.

Steps (ii)-(iv) were repeated until the final elongation process is completed.

(vi) Deprotection of the Cbz Group.

1 ml of 2:2:6 TFA/meta-Cresol/DMS solution and 1 ml of 9:1 TFA/TFMSA solution were prepared, cooled to 0° C. for several minutes, and mixed together cold, and 0.8 ml of the solution obtained was added to the resin obtained before and incubated at RT for 1 h. The resin was washed with TFA three times.

(vii) Detachment of PcPNA from the Resin.

1 ml of a 1:8:1 TFMSA/TFA/meta-Cresol solution was prepared and cooled until it turns dark green. 0.8 ml of this solution was added to the resin and incubated at RT for 1 h. The solution was transferred to a micron tube, the resin was washed with 2 ml of TFA twice, and the washings were added to the micro tube. 8 nil of diethyl ether was added to the tubes and centrifuged at 4500 RPM for 20 min at 0° C. The supernatants were removed. The procedure repeated twice.

The MS analysis of the polymers synthesized is summarized in Table 6. Polymers B, C, E, H and H13 were tested in low resolution mode, while L14 was tested in high resolution mode.

TABLE 6
Expected and found Mw/Z of the polymers synthesized

| Sequence | Z | Mw/Z expected | Mw/Z found |
|---|---|---|---|
| B | 2 | 900.41 | 900.63 |
| | 3 | 600.61 | 600.75 |
| | 4 | 450.71 | 450.81 |
| C | 2 | 1022.77 | 1022.89 |
| | 3 | 767.32 | 767.16 |
| E | 3 | 581.94 | 581.82 |
| | 4 | 436.71 | 436.87 |
| H | 2 | 1124.95 | 1124.44 |
| | 3 | 750.30 | 749.82 |
| | 5 | 450.59 | 450.31 |
| H13 | 2 | 1347.58 | 1347.20 |
| | 3 | 898.73 | 898.54 |
| | 4 | 674.30 | 674.15 |
| | 5 | 539.64 | 539.52 |
| | 6 | 449.87 | 449.77 |
| L14 | 2 | 1583.67 | 1583.68 |
| | 3 | 1056.122 | 1056.125 |
| | 4 | 795.344 | 792.345 |
| | 5 | 634.077 | 634.077 |

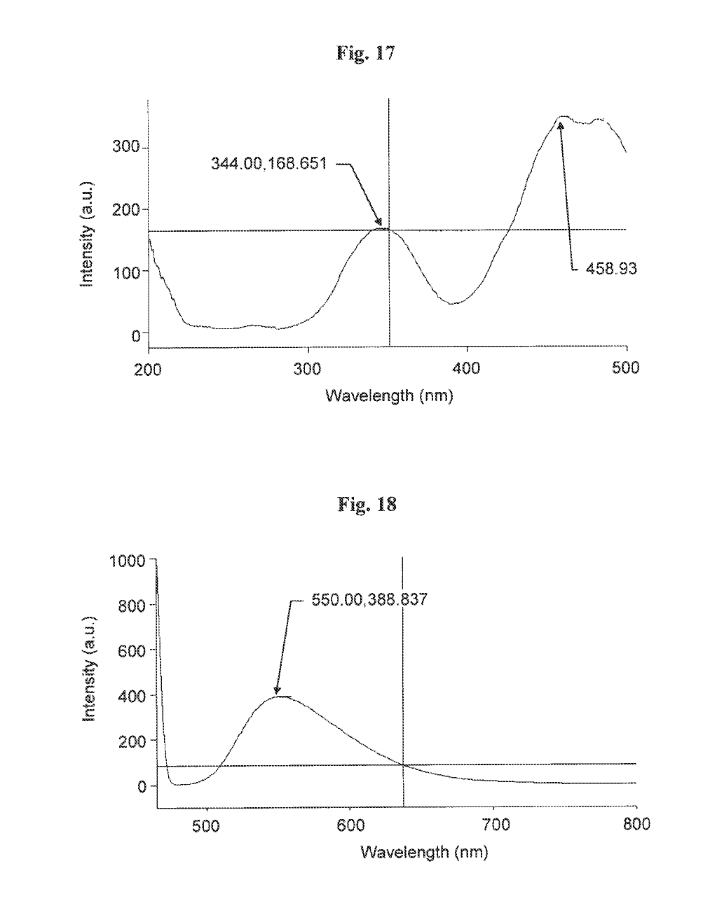

Polymers C and L14 were tested for fluorescence by UV and found to be excited with wavelength 460 nm and fluorescent in 550 nm, indicating that such polymers, when including fluorescent groups, are fluorescent and thus suitable for permeability tests. FIG. 17 shows the absorption (excitation) spectrum of polymer L14, and FIG. 18 shows the fluorescence (emission) spectrum of polymer L14 (excitation wavelength of 460 nm).

The biological activity of the polymers synthesized, i.e., their ability to specifically interact with specific fragments of double-stranded DNA, was tested using the RT-PCR method, as described in Materials and Methods.

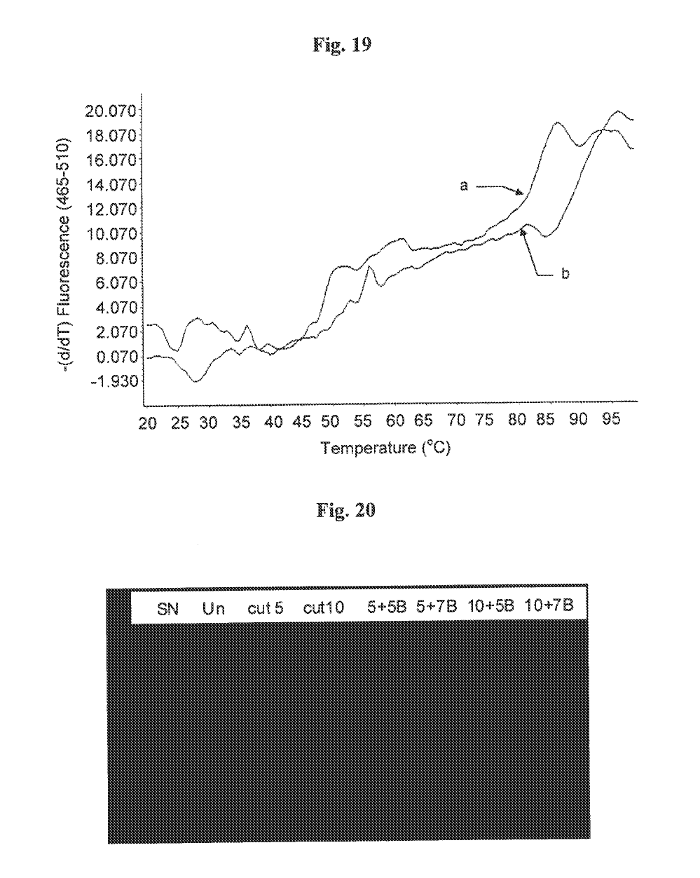

Polymer E—The 25 mer C-G DNA fragment (primers 1+3 in Table 4) was treated with polymer E, designed to be selective to DNA fragments including the sequence GGGCC/CCGGG. As found, polymer E increased the T_{M} of this DNA fragment from 86° C. in the control well to 96° C. in the treated well, as shown in FIG. 19. Two other DNA fragments that do not include the targeted sequence have not shown any increase in T_{M} upon treatment with this polymer.

It is expected that the efficacy of such a polymer in increasing the T_{M} of a target double-stranded DNA sequence, thus increasing the energy required so as to dissociate the nucleic acid molecule into two separate strands, would be substantially higher than that of a mixture of the monomers composing said polymer.

It is further expected that polymers comprising a chain of units positively charged under physiological conditions, linked via a linker to the N-terminus and/or C-terminus of the polymer, such as in polymers B, C, H13 and L14, will be capable of better linking to the target double-stranded DNA sequence, due to the electrostatic interactions between the positive charges on said units and the phosphoric groups of the target DNA backbone, compared to corresponding polymers having the same polymeric structure but without the positively charged units preceding or following the monomer units chain.

### Example 15

### Sequence Specific Double-Stranded DNA Binding Compounds Prevent Restriction Enzymes from Digesting DNA

In a preliminary study summarized in Table 7, compound AH-13, an analog of a monomer unit having a pharmacophore capable of specifically interacting with the A-T or T-A base pair in a double-stranded DNA fragment, was shown to be capable in a concentration dependent manner of preventing a restriction enzyme (satI) specific for a restriction site comprised within the sequence of said DNA fragment from digesting the DNA fragment (FIG. 20). This can be seen by comparing lanes “5+5B”, “5+7B” and “10+78” with “cut 5” and “cut 10”, wherein no AH-13 was used. Note that in lane “10+5B”, the concentration of AH-13 was not sufficient to completely interfere with the enzyme activity, as seen by the presence of both cut and uncut DNA.

TABLE 7
Protocol of experiment with satI and AH-13

| | | | | 5 + | 5 + | 10 + | 10 + |
|---|---|---|---|---|---|---|---|
| | Un | Cut5 | Cut10 | 5B | 7B | 5B | 7B |
| PCR product (μl) | 10 | 5 | 10 | 5 | 5 | 10 | 10 |
| Buffer (μl) | 3 | 3 | 3 | 3 | 3 | 3 | 3 |
| AH-13 (μl) | — | — | — | 5 | 7 | 5 | 7 |
| H₂O (μl) | 17 | 20.5 | 15.5 | 15.5 | 13.5 | 10.5 | 8.5 |
| SatI enzyme (μl) | — | 1.5 | 1.5 | 1.5 | 1.5 | 1.5 | 1.5 |
| Total (μl) | 30 μl | 30 μl | 30 μl | 30 μl | 30 μl | 30 μl | 30 μl |

In view of the preliminary results described above, a further study is conducted to show the capability of a sequence specific double-stranded DNA binding compound designed for specifically interacting with a target DNA sequence to prevent a restriction enzyme specific for a restriction site comprised within said target sequence from digesting a double-stranded DNA fragment containing said target. The protocol of this study is described herein:

### (i) PCR Amplification Condition of the Chimera BCR-ABL Gene Connection Site

Reaction Mixture

Using Thermo Scientific 2X ReddyMix PCR Master Mix w/ 1.5 mM MgCl₂

| | Volume | Final Concentration 1X |
|---|---|---|
| ReddyMix PCR Master Mix | 12.5 μl | 1X |
| Primer forward (10 μM each) | 1.25 μl | 0.5 μM |
| Primer reverse (10 μM each) | 1.25 μl | 0.5 μM |
| Water (PCR Grade) to 25 μl | | |
| DNA template 0.5-10 μl | | 0.5-125 ng |
| Total volume 25 μl | | |

* template DNA—synthetic sequence of the plasmid chimera BCR-ABL gene.

PCR Condition

PCR analysis (94° C. for 5 min; 40 cycles of 94° C. for 30 sec; 58° C. for 30 sec; 72° C. for 30 sec; and final elongation 72° C. for 5 min) with the following primers which yield a 251-bp DNA fragment: 5′-GTCTTCCTGTTCACCGACCT-3′ (forward primer; SEQ ID NO: 9) and 5′-GTAGCCTTGCTGCCCTTGT-3′ (reverse primer; SEQ ID NO: 10).

### (ii) Restriction Digest of Chimera BCR-ABL PCR Product

In this step, we take our PCR product (251 bp) and digest it with a restriction enzyme (SatI) which cut two sites in the PCR product. The restriction site sequence is:

. . . GCNGC . . .
. . . CGNCG . . .,

wherein N represents A, T, C or G.

The restriction site sequence after cut:

. . . GC NGC . . .
. . . CGN CG . . .

The first digestion site is at the connection site between the two genes BCR and ABL; and the second site is at the end of the product to give 176-bp, 64-bp and 11-bp.

(iii) Digestion BCR-ABL PCR Product Procedure

Combine 3 μl 10× buffer, 10 μl H₂O/chemical solution, 15.5 μl PCR product, 1.5 μl enzyme (total—30 μl) in a microfuge tube in order; and incubate for 2 hour at 37° C. in a water-bath. Meanwhile prepare the agarose gel.

### (iv) Analysis

In order to visualize the samples, add 6 μl of 10× load dye directly to the digest reaction, and run the 36 μl on a 3% agarose gel.

### Example 16

### The Biological Activity of Triplex Forming Molecules of the Invention on Double-Stranded DNA Sequences

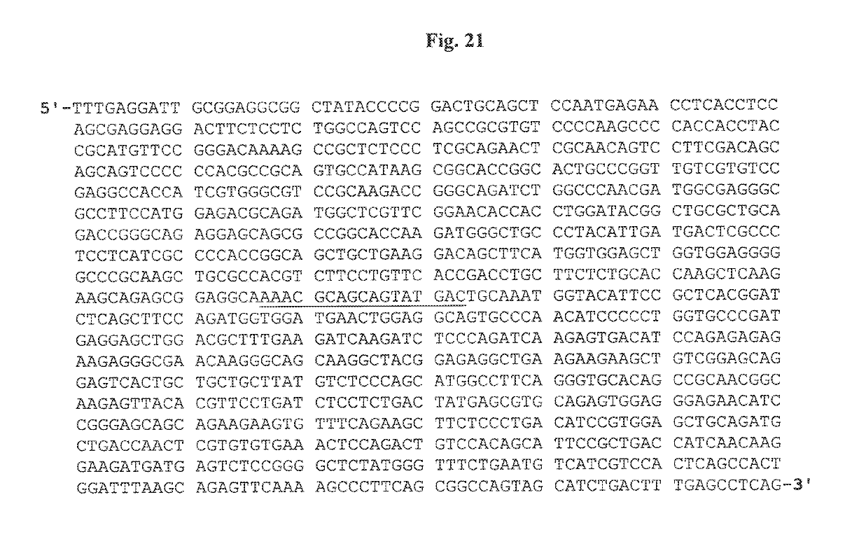

In this experiment, a natural dsDNA fragment of the chimera BCR-ABL gene was synthesized by fly-lab Ltd. (Israel). The BCR-ABL gene is responsible for chronic myelogenous leukemia (CML). More particularly, in CML patients, the Philadelphia chromosome comprises a gene termed P210BCR-ABL, which is constitutively expressed producing activated nonreceptor tyrosine kinase, an oncoprotein that causes cell transformation by phosphorylation of signaling molecules. The specific sequence for targeting selected in this case was chosen so as to have maximum mismatches with other genes and is shown in FIG. 21. This sequence is 5′-AAACGCAGCAGTATGAC-3′ (SEQ ID NO: 11) (3′-TTTGCGTCGTCATACTG-5′, SEQ ID NO: 12), which comprises at least 3 mismatches (underlined) to the closest other genes in the human genome (the closest stretch along the human genome belongs to Homo sapiens zinc finger protein 407 (ZNF407), RelSeqGene on chromosome 18, having the sequence 5′-taacgcagcagtatcaa-3′).

In order to test the biological activity of triplex forming molecules of the invention on the BCR-ABL fragment selected, the T_{M} of the BCR-ABL fragment is tested using UV and/or RT-PCR techniques as described in Materials and Methods. The BCR-ABL fragment is then incubated with a corresponding triplex forming molecule of the invention, designed so as to fit the sequence of the BCR-ABL fragment selected, after which the effect of the triplex forming molecule on the T_{M} of the fragment is tested. In addition, the amplification of the chosen BCR-ABL fragment, following incubation with the triplex forming molecule of the invention is tested using PCR, so as to verify whether the triplex forming molecule indeed prevents amplification of the fragment.

The translation in vitro of the selected BCR-ABL fragment, following incubation with the triplex forming molecule, is tested using EasyXpress®Protein Synthesis kit (Qiagen, USA), which uses highly productive E. coli lysates that contain all translational machinery components, i.e., ribosomes, ribosomal factors, tRNAs, aminoacyl-tRNA synthetases, etc.) as well as T7 RNA polymerase. The kit further contains reaction buffers, amino acid mix without methionine, methionine, RNase-free water, gel-filtration columns, and reaction flasks.

Before carrying out this procedure, it should be noted that (i) the plasmid DNA expression template encoding the protein of BCR-ABL selected fragment must contain a T7 or other strong E. coli promoter and a ribosome binding site. The plasmid is designed to have the coding region 6×His tag, which can be synthesized with the proteins and utilized for later purification using Ni-NTA Superflow; (ii) this in vitro translation system is extremely sensitive to nuclease contamination and therefore. RNase- and DNase-free reaction tubes should be used; (iii) all handling steps using E. coli extracts for the protein synthesis or the translation reaction of BCR-ABL fragment should be carried out on ice; and (iii) the recommended incubation temperature for protein synthesis is 37° C.

The in vitro procedure is carried out according to the following protocol:

Initial In Vitro Synthesis Reaction
(1) Thaw and store E. coli extract, methionine, feeding solution, and energy mix on ice. Thaw RNase-free water and equilibration/elution buffer at RT (15-25° C.);
(2) Thaw reaction buffer (-methionine) in the supplied 12 ml plastic tube on ice and vortex thoroughly;
(3) Add 100 μl of a 60 mM solution of methionine to the reaction buffer in the 12 ml plastic tube, which will serve as the reaction vessel for the initial protein synthesis reaction;
(4) Add 50 pmol of plasmid DNA expression template encoding the protein of BCR-ABL selected fragment to the reaction buffer. This corresponds to a final concentration of 10 nM (100 μg of a 3 kb plasmid) in the final 5 ml reaction volume;
(5) Make up the reaction volume to 3.25 ml with RNase-free water;
(6) Add 1.75 ml E. coli extract to the reaction;
(7) Gently mix the reaction by pipetting up and down;
(8) Incubate the reaction in a water-bath at 37° C. with gentle shaking for 1 h;
(9) Immediately after starting protein synthesis reaction, prepare and equilibrate a gel filtration column, i.e., unscrew and remove the bottom closure and peel off the top seal; allow the storage buffer to drain out; equilibrate the column by applying 3×17 ml aliquots of equilibration buffer and allowing the buffer to flow through the column (this step as well as the following steps 10-13 can be performed at RT;
(10) After 1 h incubation (step 8), centrifuge the tube containing the protein synthesis reaction at 10,000×g for 3 min;
(11) Carefully pipet the entire supernatant from step 10 onto the equilibrated gel filtration column;
(12) After the supernatant has entered the column, pipet 1 ml equilibration/elution buffer onto the column. Discard the flowthrough fraction; and
(13) Place a 50 ml reaction flask under the column and pipet 7 ml equilibration/elution buffer onto the column. Collect the flowthrough fraction in the reaction flask, which will serve as the reaction vessel for the second protein synthesis reaction. The flow-through fraction contains the recycled high molecular-weight reaction components.

Second In Vitro Synthesis Reaction
(14) Add 200 μl of a 60 mM solution of methionine to the protein synthesis reaction (flowthrough fraction from step 13);
(15) Thoroughly vortex the tube containing feeding solution and add 1700 μl to the protein synthesis reaction (there may be a precipitate visible in the tube containing feeding solution. This will not adversely affect the reaction);
(16) Add 1100 μl energy mix to the protein synthesis reaction;
(17) Gently mix the reaction by pipetting up and down;
(18) Incubate the reaction in a water-bath at 37° C. with gentle shaking for 1 h;
(19) In vitro-synthesized protein of BCR-ABL fragment that carry a 6×His tag can be easily purified using Ni-NTA superflow; and
(20) Determine the translation or the BCR-ABL protein synthesis in the presence and absence of the triplex forming molecules, using Western blots techniques. The reaction products are separated by gel electrophoresis using 12% polyacrylamide gels and then transferred to nitrocellulose membrane. The membrane is incubated with anti-6His tag antibody conjugated to HRP, which is supposed to bind to the synthesized BCR-ABL chimera carrying the 6His tag on the N-terminal end. The blot is analyzed using the ECL method.

### Cell Free Experiments

In this experiment, the effect of a triplex forming molecule of the invention, specific to BCR-ABL, on the expression of BCR-ABL gene in a CML cell line is tested. The cytotoxicity of the triplex forming molecule is measured both in cells of CML cell line and in normal cells, and the expression of the CML gene producing the BCR-ABL protein is measured by Western blots, so as to verify whether the triplex forming molecule can inhibit the CML gene expression and consequently the BCR-ABL protein production.

This experiment is carried out according to the following protocol:
(1) Grow cells in their appropriate media;
(2) Isolate the proteins and resolve on gel electrophoresis;
(3) Transfer the proteins to nitrocellulose membrane and conduct Western blot using antibody specific to BCR-ABL protein. Make sure that the BCR-ABL protein is expressed in this cell line;
(4) Test the effect of a triplex forming molecule specific to BCR-ABL gene on its expression using Western blot. High concentration of triplex forming molecule may be toxic to the cells;
(5) Labeled triplex forming molecule can be used to determine the uptake into cells.

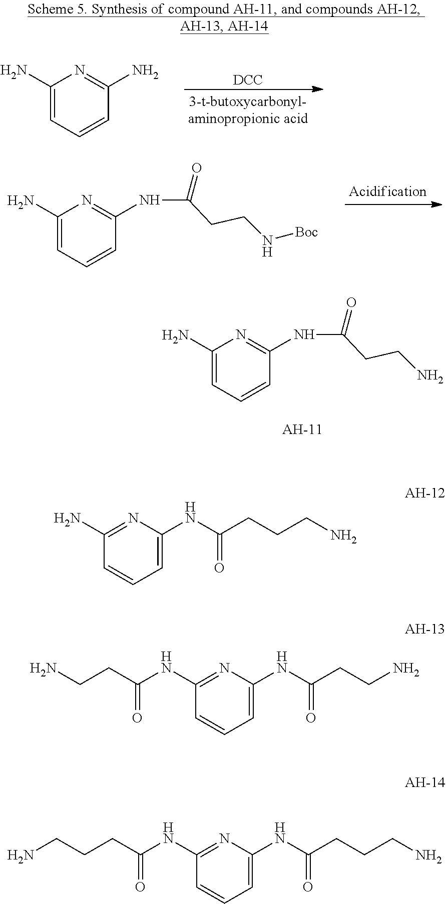

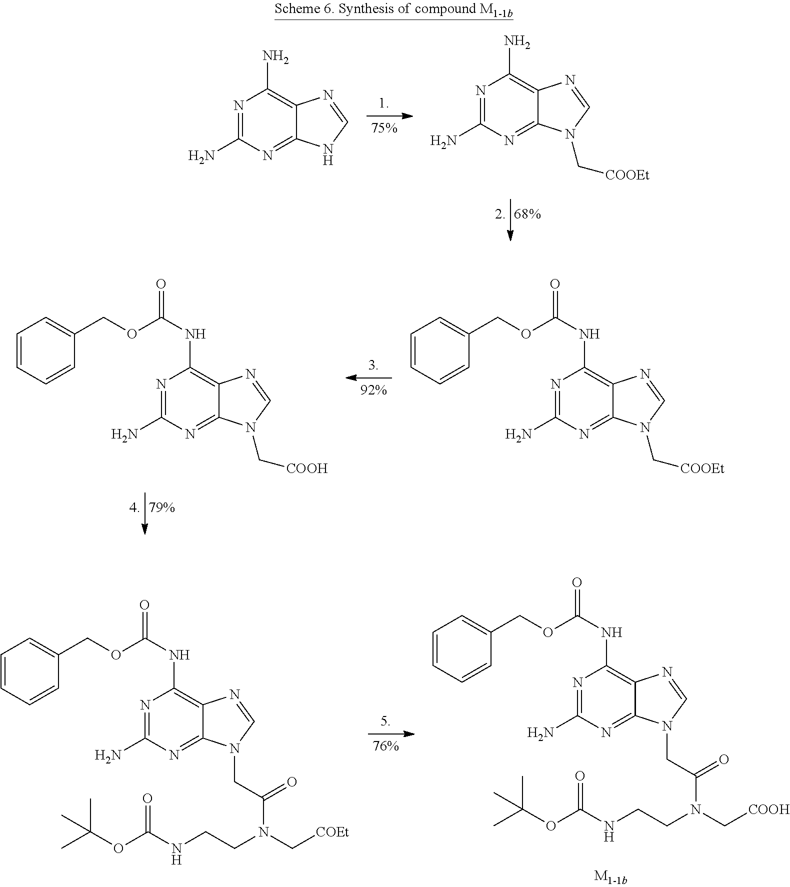

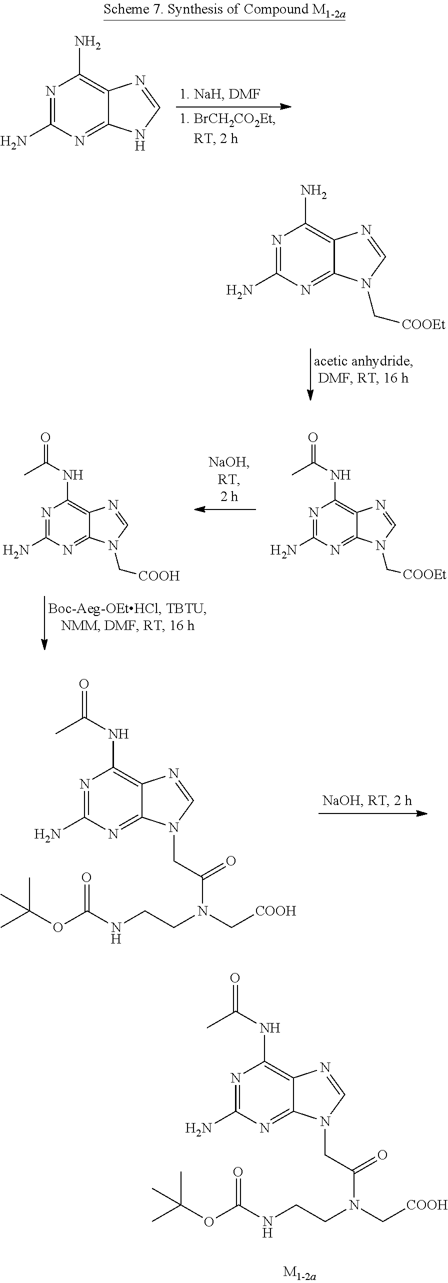 [SMILES: Nc1nc(N)c2nc[nH]c2n1 ; CCOC(=O)Cn1cnc2c(N)nc(N)nc21 ; CC(=O)Nc1nc(N)nc2c1ncn2CC(=O)O ; CCOC(=O)Cn1cnc2c(NC(C)=O)nc(N)nc21 ; CC(=O)Nc1nc(N)nc2c1ncn2CC(=O)N(CCNC(=O)OC(C)(C)C)CC(=O)O ; CC(=O)Nc1nc(N)nc2c1ncn2CC(=O)N(CCNC(=O)OC(C)(C)C)CC(=O)O]

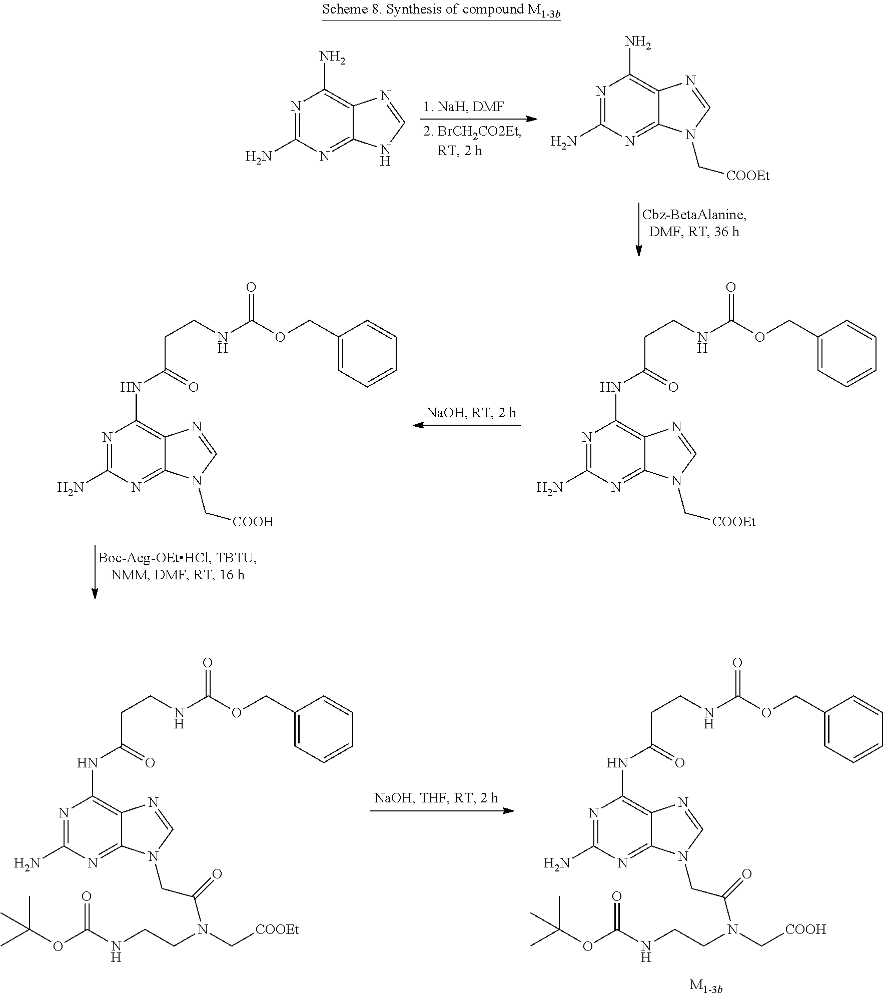 [SMILES: Nc1nc(N)c2nc[nH]c2n1 ; CCOC(=O)Cn1cnc2c(N)nc(N)nc21 ; Nc1nc(NC(=O)CCNC(=O)OCc2ccccc2)c2ncn(CC(=O)O)c2n1 ; CCOC(=O)Cn1cnc2c(NC(=O)CCNC(=O)OCc3ccccc3)nc(N)nc21 ; CCOC(=O)CN(CCNC(=O)OC(C)(C)C)C(=O)Cn1cnc2c(NC(=O)CCNC(=O)OCc3ccccc3)nc(N)nc21 ; CC(C)(C)OC(=O)NCCN(CC(=O)O)C(=O)Cn1cnc2c(NC(=O)CCNC(=O)OCc3ccccc3)nc(N)nc21]

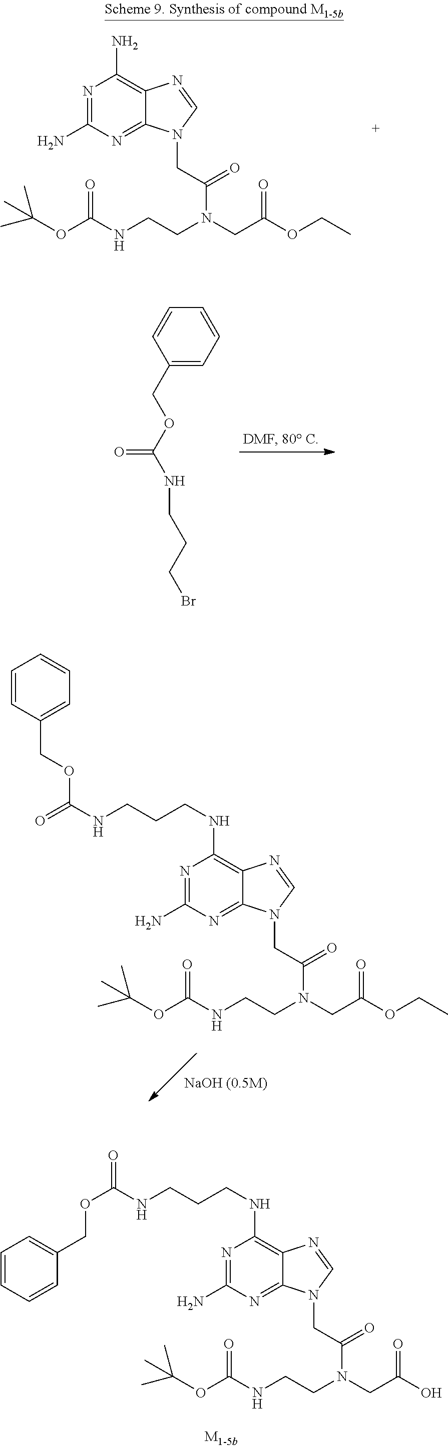 [SMILES: CCOC(=O)CN(CCNC(=O)OC(C)(C)C)C(=O)Cn1cnc2c(N)nc(N)nc21 ; O=C(NCCCBr)OCc1ccccc1 ; CCOC(=O)CN(CCNC(=O)OC(C)(C)C)C(=O)Cn1cnc2c(NCCCNC(=O)OCc3ccccc3)nc(N)nc21 ; CC(C)(C)OC(=O)NCCN(CC(=O)O)C(=O)Cn1cnc2c(NCCCNC(=O)OCc3ccccc3)nc(N)nc21]

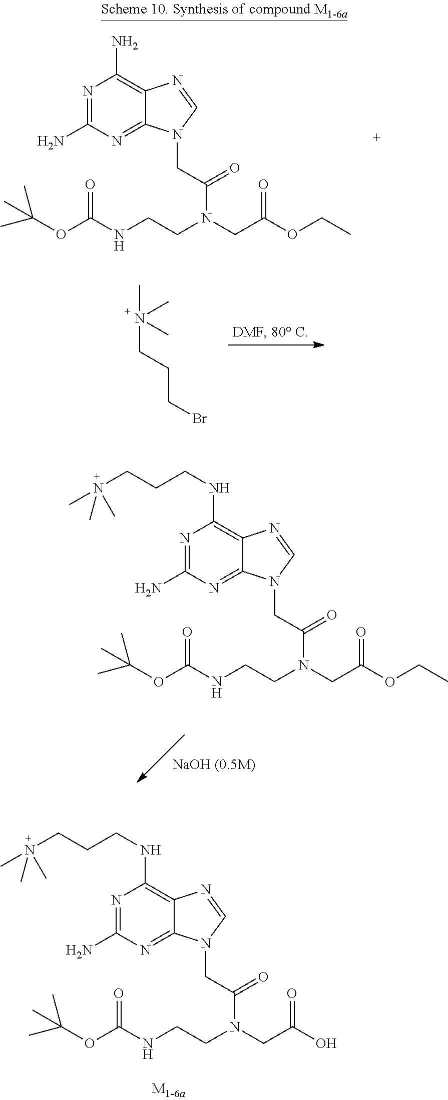

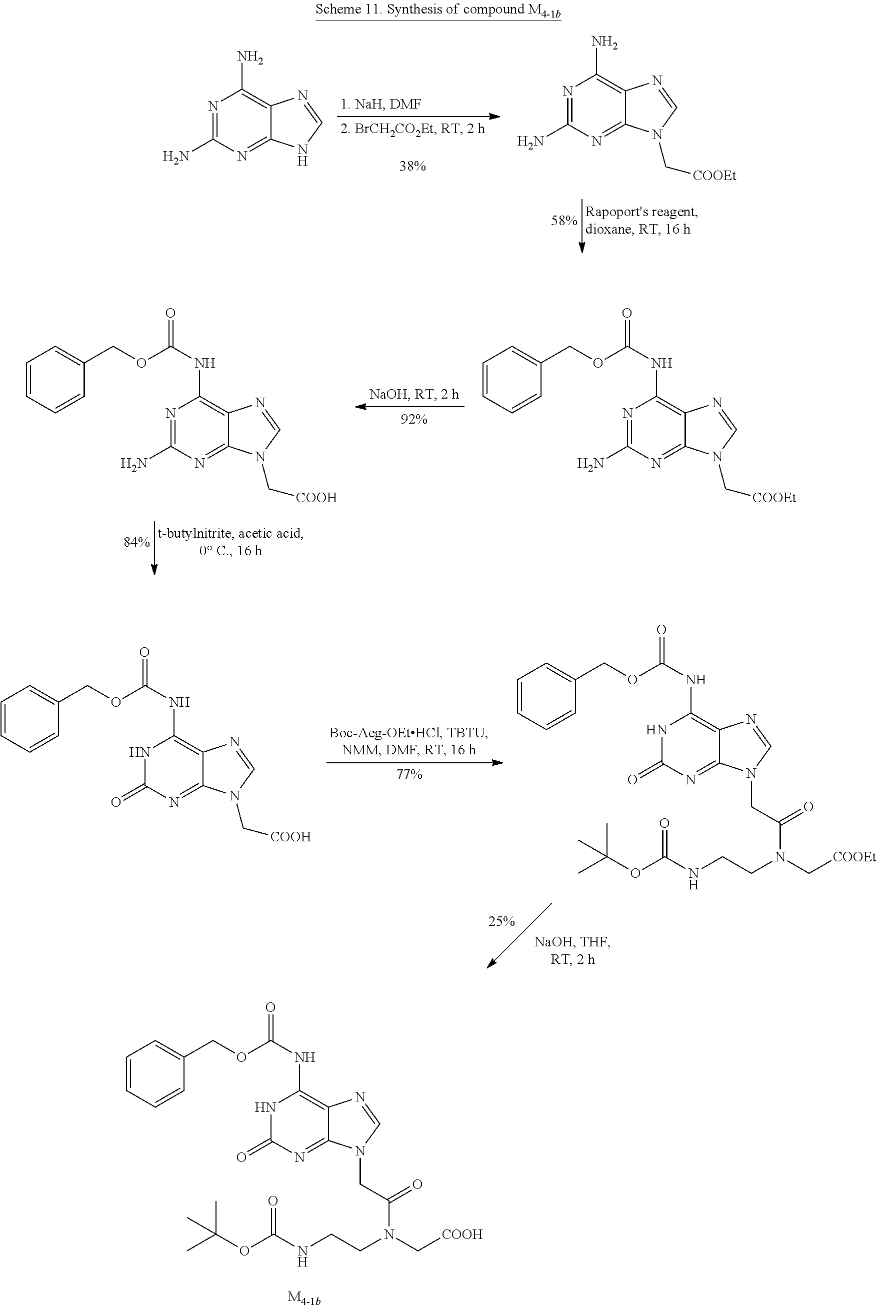

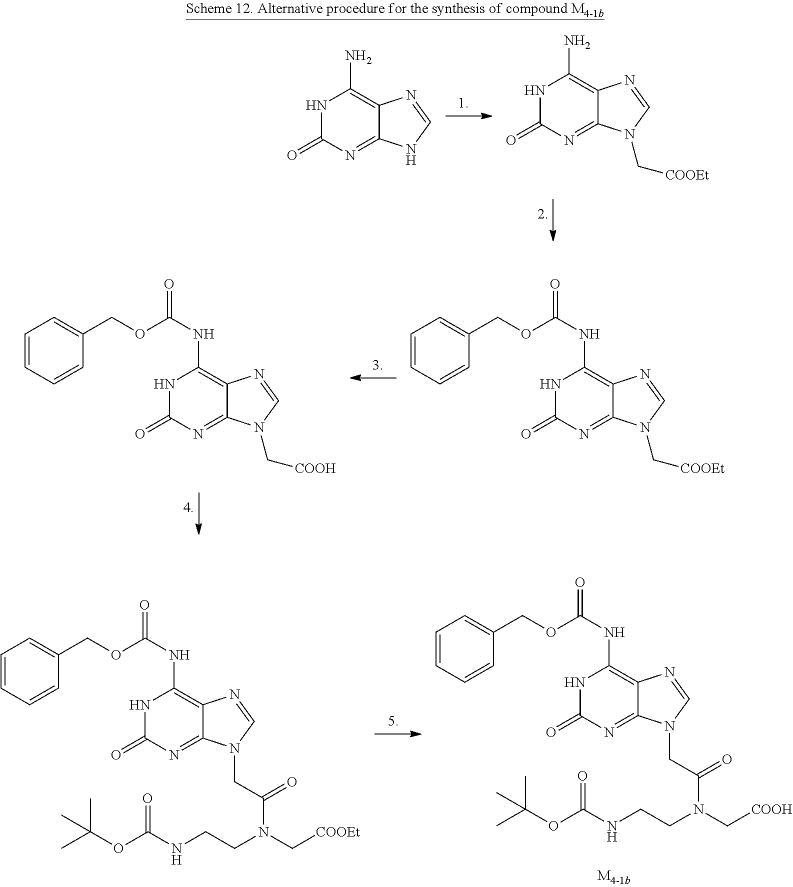 [SMILES: Nc1[nH]c(=O)nc2[nH]cnc12 ; CCOC(=O)Cn1cnc2c(N)[nH]c(=O)nc21 ; O=C(O)Cn1cnc2c(NC(=O)OCc3ccccc3)[nH]c(=O)nc21 ; CCOC(=O)Cn1cnc2c(NC(=O)OCc3ccccc3)[nH]c(=O)nc21 ; CCOC(=O)CN(CCNC(=O)OC(C)(C)C)C(=O)Cn1cnc2c(NC(=O)OCc3ccccc3)[nH]c(=O)nc21 ; CC(C)(C)OC(=O)NCCN(CC(=O)O)C(=O)Cn1cnc2c(NC(=O)OCc3ccccc3)[nH]c(=O)nc21]

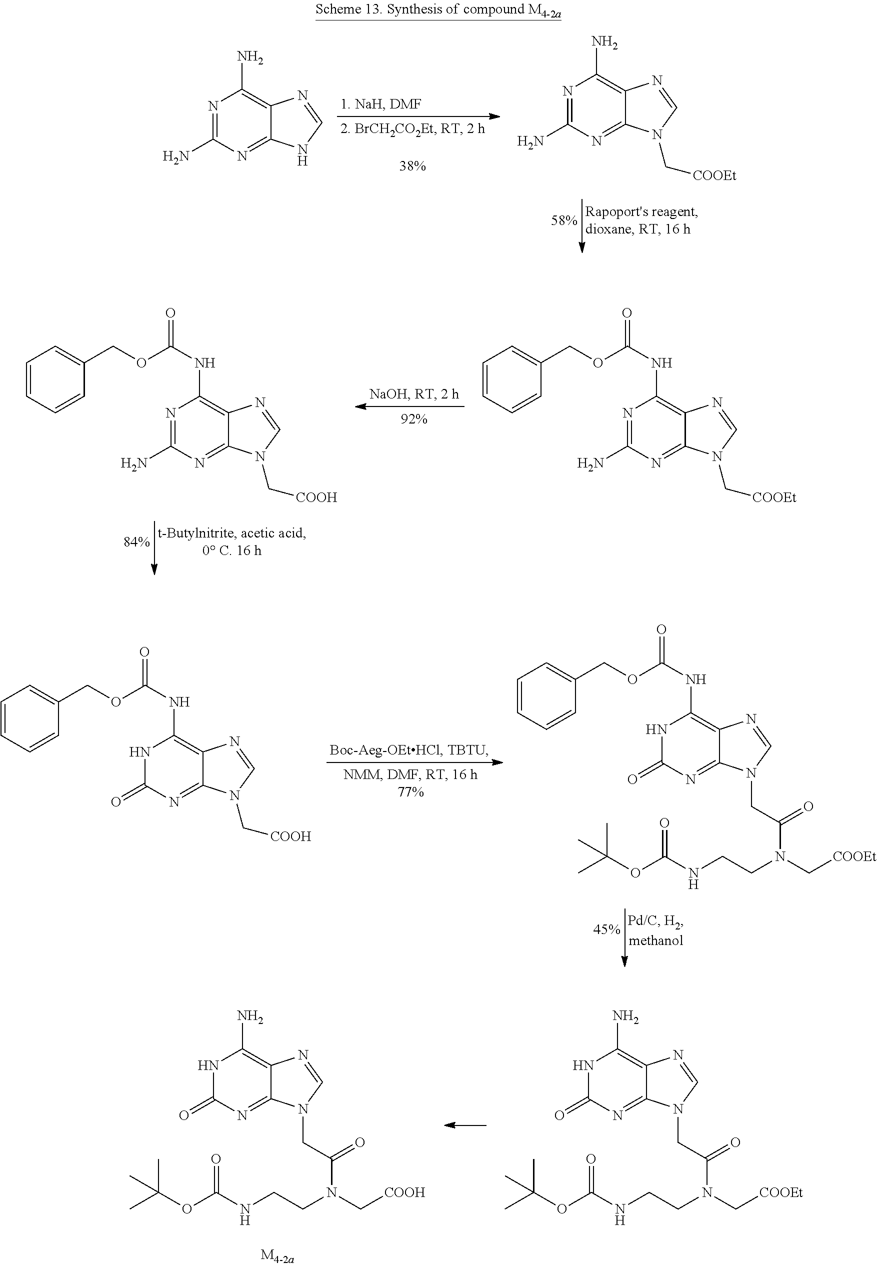

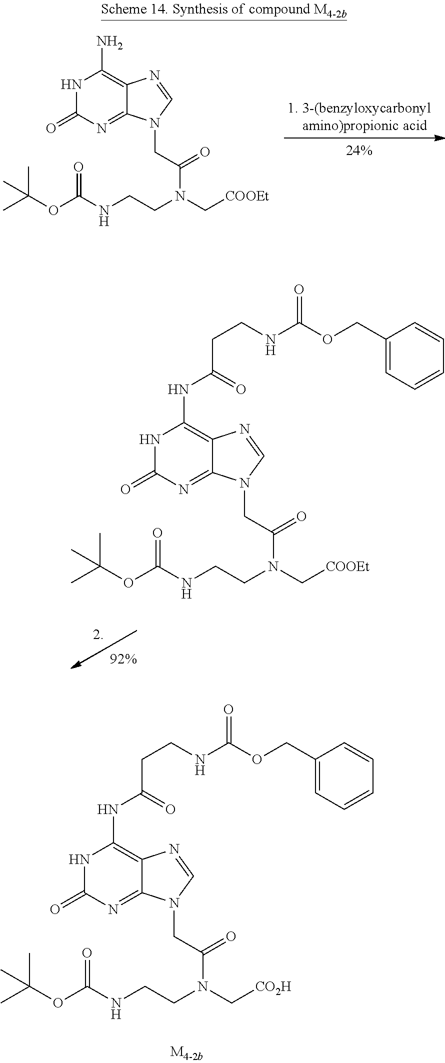 [SMILES: CCOC(=O)CN(CCNC(=O)OC(C)(C)C)C(=O)Cn1cnc2c(N)[nH]c(=O)nc21 ; CCOC(=O)CN(CCNC(=O)OC(C)(C)C)C(=O)Cn1cnc2c(NC(=O)CCNC(=O)OCc3ccccc3)[nH]c(=O)nc21 ; CC(C)(C)OC(=O)NCCN(CC(=O)O)C(=O)Cn1cnc2c(NC(=O)CCNC(=O)OCc3ccccc3)[nH]c(=O)nc21]

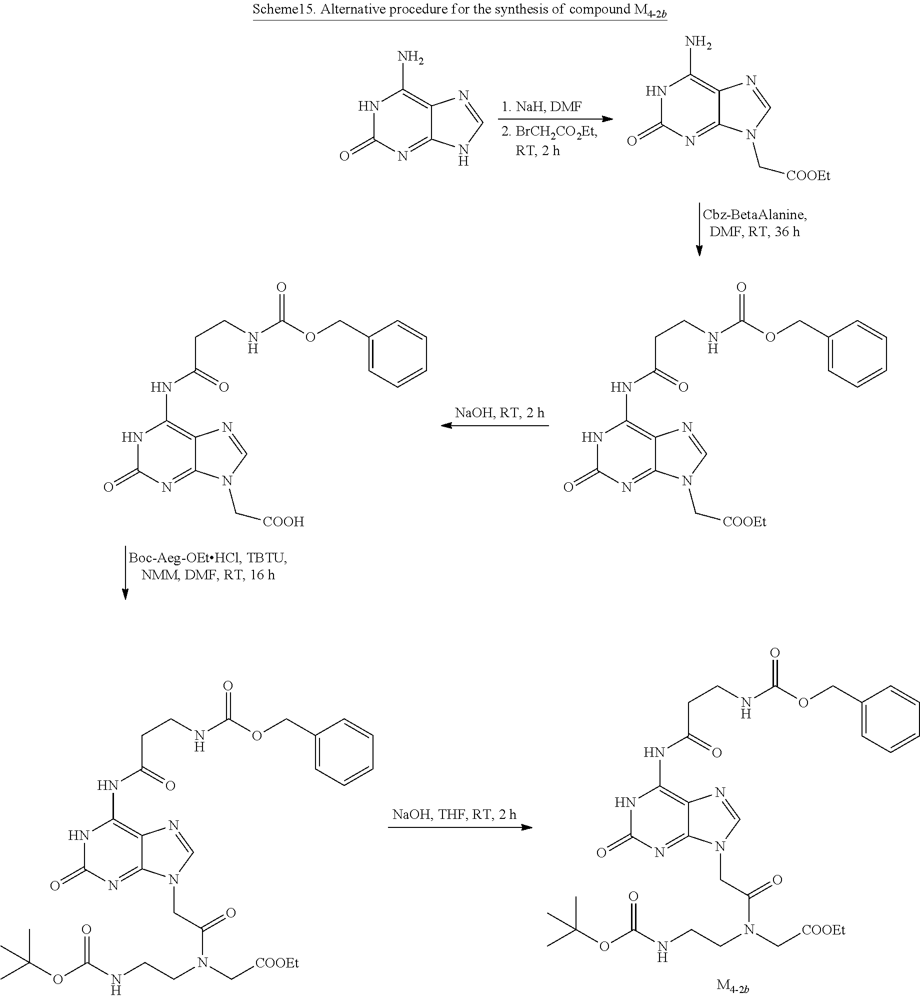 [SMILES: Nc1[nH]c(=O)nc2[nH]cnc12 ; CCOC(=O)Cn1cnc2c(N)[nH]c(=O)nc21 ; O=C(O)Cn1cnc2c(NC(=O)CCNC(=O)OCc3ccccc3)[nH]c(=O)nc21 ; CCOC(=O)Cn1cnc2c(NC(=O)CCNC(=O)OCc3ccccc3)[nH]c(=O)nc21 ; CCOC(=O)CN(CCNC(=O)OC(C)(C)C)C(=O)Cn1cnc2c(NC(=O)CCNC(=O)OCc3ccccc3)[nH]c(=O)nc21 ; CCOC(=O)CN(CCNC(=O)OC(C)(C)C)C(=O)Cn1cnc2c(NC(=O)CCNC(=O)OCc3ccccc3)[nH]c(=O)nc21]

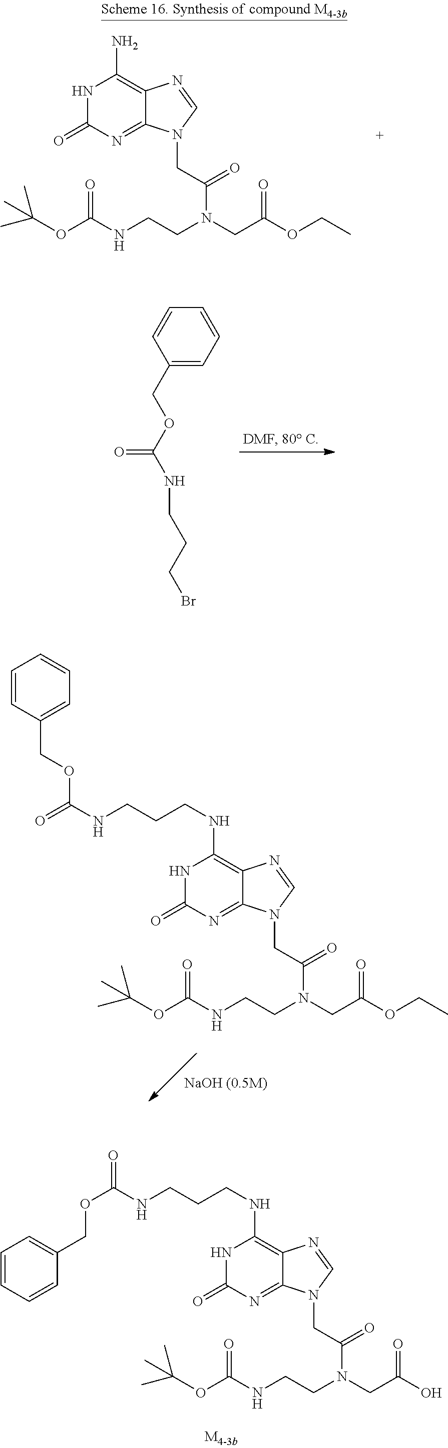 [SMILES: CCOC(=O)CN(CCNC(=O)OC(C)(C)C)C(=O)Cn1cnc2c(N)[nH]c(=O)nc21 ; O=C(NCCCBr)OCc1ccccc1 ; CCOC(=O)CN(CCNC(=O)OC(C)(C)C)C(=O)Cn1cnc2c(NCCCNC(=O)OCc3ccccc3)[nH]c(=O)nc21 ; CC(C)(C)OC(=O)NCCN(CC(=O)O)C(=O)Cn1cnc2c(NCCCNC(=O)OCc3ccccc3)[nH]c(=O)nc21]

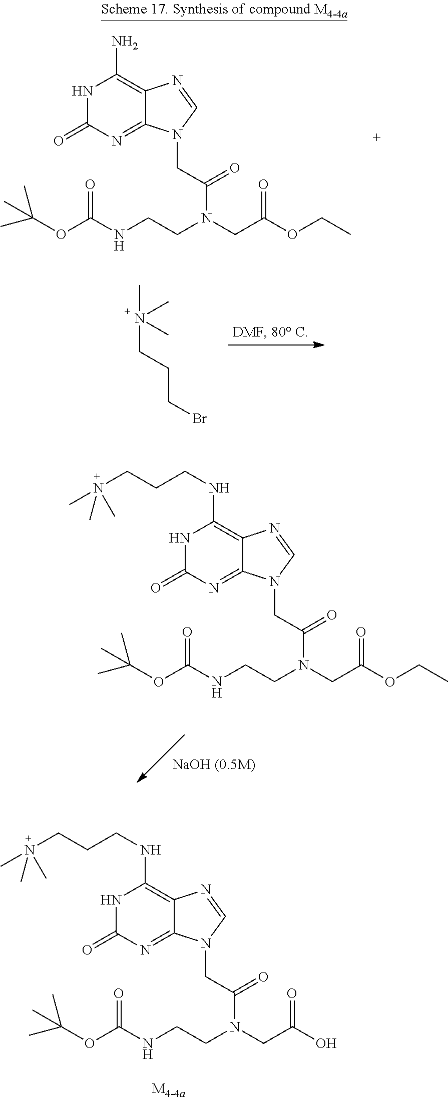

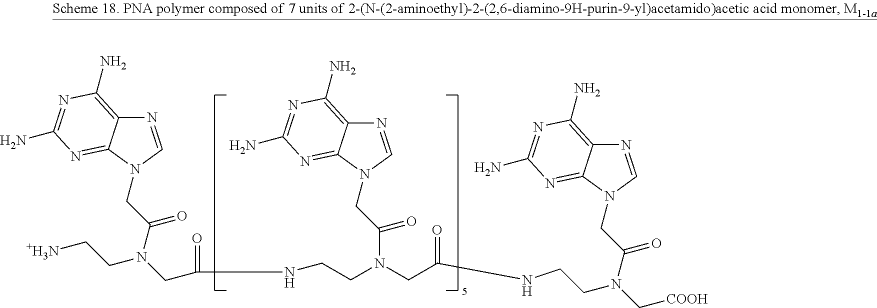

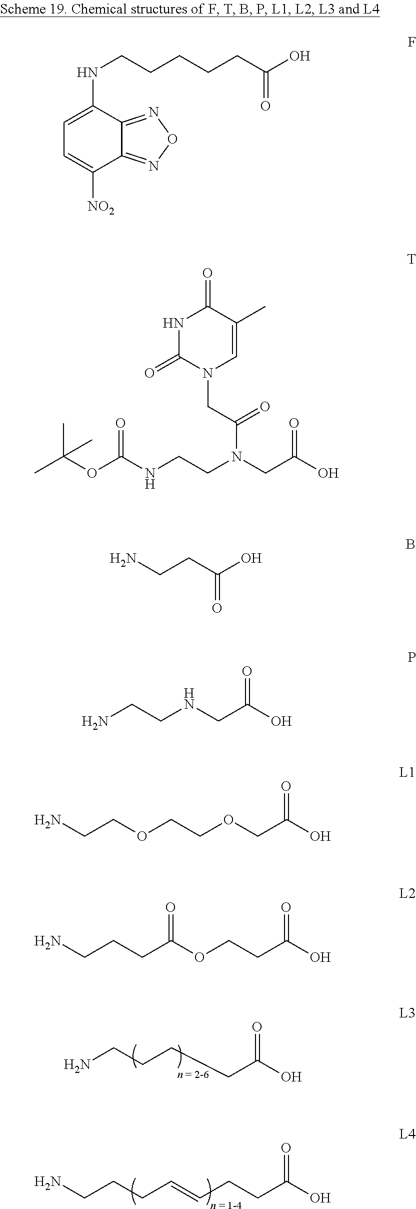

1-29. (canceled)

30. A monomer unit of the general formula Im: 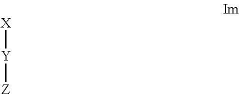
wherein
Z is a monomer of the formula IIm, IIIm, or IVm: 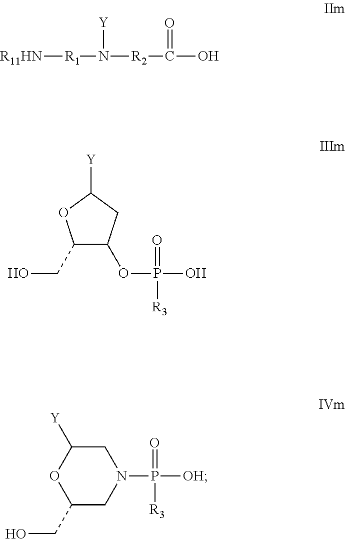
Y is a covalent bond or a linker selected from the group consisting of —CR′₂—CO—, —CR′₂—CS—, and —(CH₂)₁₋₆— optionally substituted with at least one functional group, wherein R′ each independently is H, halogen, or a (C₁-C₃)alkyl optionally substituted with at least one functional group; and
X is a chemical moiety of a formula selected from the group consisting of the formulas X₁-X₁₃: 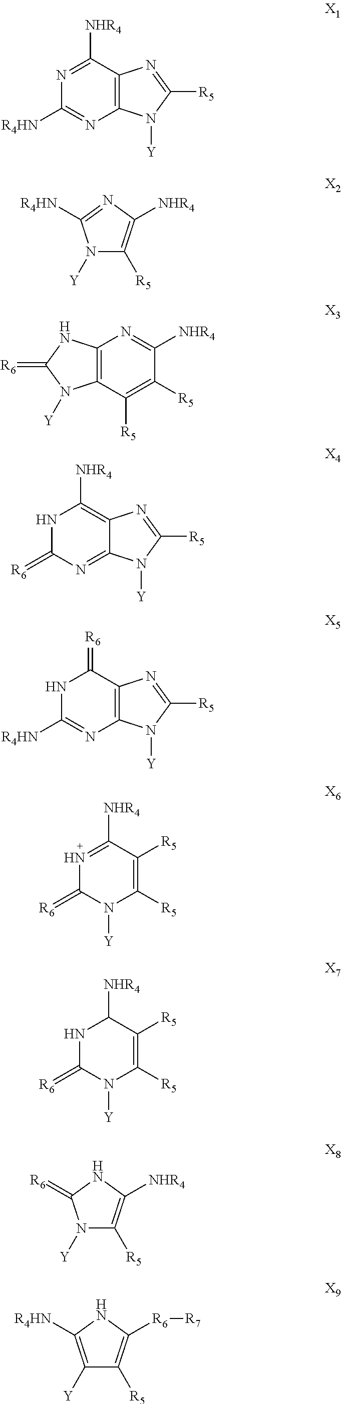 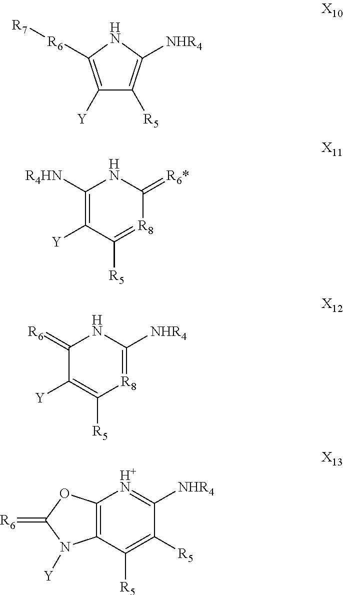
wherein
R₁ is —(CH₂)₁₋₃—, or R₁ together with the nitrogen atom of the secondary amine linked thereto form a 5-6-membered heterocyclic ring;
R₂ is —(CH₂)₁₋₃—;
R₃ is —O⁻, —OH, —OR″, —S⁻, —SH, —SR″, —NR″₂ or a (C₁-C₅)alkyl optionally substituted with at least one functional group, wherein R″ each independently is H, halogen, or a (C₁-C₅)alkyl optionally substituted with at least one functional group;
R₄ each independently is —COR₉, R₉ or R₁₁;
R₅ each independently is H, halogen, —NH₂, (C₁-C₅)alkyl optionally interrupted with a heteroatom selected from the group consisting of O, S or N, or —S—(C₁-C₅)alkyl;
R₆ is O or S;
R₇ is —COR₉ or R₉;
R₈ is CH or N;
R₉ is (C₁-C₃)alkyl, (C₂-C₃)alkenyl, —(CH₂)₁₋₃NHR₁₀, —(CH₂)₁₋₃N(R₁₀)₃⁺, or a 5-6-membered nitrogen containing heterocyclic ring wherein the nitrogen is optionally further substituted with a (C₁-C₃)alkyl;
R₁₀ each independently is a (C₁-C₃)alkyl or R₁₁;
R₁₁ each independently is H or an amine protecting group; and
said functional group is selected from the group consisting of free amino, carboxyl or hydroxyl,
but excluding the monomer units wherein Z is a monomer of the formula IIm, wherein R₁ is —(CH₂)₂—, and R₂ is —CH₂—; Y is —CR′₂—CO—; and (i) X is X₁, wherein R₄ each independently is H or an amine protecting group, and R₅ is H; (ii) X is X₅, wherein R₄ is H or an amine protecting group, R₅ is H, and R₆ is O; or (iii) X is X₆, wherein R₄ is H or an amine protecting group, R₅ is H, and R₆ is O.

31. The monomer unit of claim 30, wherein Y is —CR′₂—CO— or —CR′₂—CS—, wherein R′ each independently is H or a (C₁-C₂)alkyl optionally substituted with at least one functional group; and Z is a monomer of the formula IIm.

32. The monomer unit of claim 31, wherein Y is —CR′₂—CO—, wherein R′ each independently is H or methyl optionally substituted with at least one functional group; and Z is a monomer of the formula IIm, wherein R₁ is —(CH₂)₂— and R₂ is —CH₂—, or R₁ is —CH₂— and R₂ is —(CH₂)₂—.

33. The monomer unit of claim 32, wherein Y is —CH₂—CO—; and Z is a monomer of the formula II, wherein R₁ is —(CH₂)₂—, R₂ is —CH₂—, and R₁₁ is t-butoxycarbonyl.

34. The monomer unit of claim 33, wherein
(i) X is a chemical moiety of the general formula X₁, wherein R₄ of the amine group linked to the carbon atom at position 2 of the purine moiety is R₁₁, wherein R₁₁ is H; R₄ of the amine group linked to the carbon atom at position 6 of the purine moiety is COR₉, wherein R₉ is methyl; and R₅ is H, herein identified monomer M₁₋₂ₐ;
(ii) X is a chemical moiety of the general formula X₁, wherein R₄ of the amine group linked to the carbon atom at position 2 of the purine moiety is R₁₁, wherein R₁₁ is H; R₄ of the amine group linked to the carbon atom at position 6 of the purine moiety is COR₉, wherein R₉ is (CH₂)₂NHR₁₀, R₁₀ is R₁₁, and R₁₁ is H or benzyloxycarbonyl; and R₅ is H, herein identified monomers M₁₋₃ₐ and M_{1-3b}, respectively;
(iii) X is a chemical moiety of the general formula X₁, wherein R₄ of the amine group linked to the carbon atom at position 2 of the purine moiety is COR₉, wherein R₉ is (CH₂)₂NHR₁₀, R₁₀ is R₁₁, and R₁₁ is H; R₄ of the amine group linked to the carbon atom at position 6 of the purine moiety is R₁₁, wherein R₁₁ is H or benzyloxycarbonyl; and R₅ is H, herein identified monomers M₁₋₄ₐ and M_{1-4b}, respectively;
(iv) X is a chemical moiety of the general formula X₁, wherein R₄ of the amine group linked to the carbon atom at position 2 of the purine moiety is R₁₁, wherein R₁₁ is H; R₄ of the amine group linked to the carbon atom at position 6 of the purine moiety is R₉, wherein R₉ is —(CH₂)₃NHR₁₀, R₁₀ is R₁₁, and R₁₁ is Cbz; and R₅ is H, herein identified monomer M_{1-5b};
(v) X is a chemical moiety of the general formula X₁, wherein R₄ of the amine group linked to the carbon atom at position 2 of the purine moiety is R₁₁, wherein R₁₁ is H; R₄ of the amine group linked to the carbon atom at position 6 of the purine moiety is R₉, wherein R₉ is —(CH₂)₃N(R₁₀)₃⁺, and R₁₀ each is CH₃; and R₅ is H, herein identified monomer M₁₋₆ₐ;
(vi) X is a chemical moiety of the general formula X₄, wherein R₄ is R₁₁, wherein R₁₁ is H or benzyloxycarbonyl; R₅ is H; and R₆ is O, herein identified monomers M₄₋₁ₐ and M_{4-1b}, respectively;
(vii) X is a chemical moiety of the general formula X₄, wherein R₄ is COR₉, wherein R₉ is (CH₂)₂NHR₁₀, R₁₀ is R₁₁, and R₁₁ is H or benzyloxycarbonyl; R₅ is H; and R₆ is O, herein identified monomers M₄₋₂ₐ and M_{4-2b}, respectively;
(viii) X is a chemical moiety of the general formula X₄, wherein R₄ is R₉, wherein R₉ is (CH₂)₃NHR₁₀, R₁₀ is R₁₁, and R₁₁ is Cbz; R₅ is H; and R₆ is O, herein identified monomer M_{4-3b}; or
(ix) X is a chemical moiety of the general formula X₄, wherein R₄ is R₉, wherein R₉ is (CH₂)₃N(R₁₀)₃⁺, and R₁₀ each is CH₃; R₅ is H; and R₆ is O, herein identified monomer M₄₋₄ₐ

35. The monomer unit of claim 30, wherein Y is a covalent bond; and Z is a monomer of the formula IIIm or IVm.

36. The monomer unit of claim 35, wherein (i) Z is a monomer of the formula IIIm, wherein R₃ is selected from the group consisting of —O⁻, —OH, —S⁻, —SH, and a (C₁-C₂)alkyl optionally substituted with at least one functional group; or (ii) Z is a monomer of the formula IVm, wherein R₃ is NR″₂ wherein R″ each independently is H or a (C₁-C₂)alkyl optionally substituted with at least one functional group.


SEQUENCE LISTING (14 sequences)

SEQ ID NO: 1 - DNA, 25 bp, Artificial Sequence
  Synthetic
gggccgggccgggccgggccgggcc

SEQ ID NO: 2 - DNA, 25 bp, Artificial Sequence
  Synthetic
ccgggccgggccgggccgggccggg

SEQ ID NO: 3 - DNA, 25 bp, Artificial Sequence
  Synthetic
ggcccggcccggcccggcccggccc

SEQ ID NO: 4 - DNA, 25 bp, Artificial Sequence
  Synthetic
cccggcccggcccggcccggcccgg

SEQ ID NO: 5 - DNA, 24 bp, Artificial Sequence
  Synthetic
gaaaaaaaaaaaaaaaaaaaaaac

SEQ ID NO: 6 - DNA, 24 bp, Artificial Sequence
  Synthetic
caaaaaaaaaaaaaaaaaaaaaag

SEQ ID NO: 7 - DNA, 24 bp, Artificial Sequence
  Synthetic
gttttttttttttttttttttttc

SEQ ID NO: 8 - DNA, 24 bp, Artificial Sequence
  Synthetic
cttttttttttttttttttttttg

SEQ ID NO: 9 - DNA, 20 bp, Artificial Sequence
  Synthetic
gtcttcctgttcaccgacct

SEQ ID NO: 10 - DNA, 19 bp, Artificial Sequence
  Synthetic
gtagccttgctgcccttgt

SEQ ID NO: 11 - DNA, 17 bp, Artificial Sequence
  Synthetic
aaacgcagcagtatgac

SEQ ID NO: 12 - DNA, 17 bp, Artificial Sequence
  Synthetic
tttgcgtcgtcatactg

SEQ ID NO: 13 - DNA, 17 bp, Artificial Sequence
  Synthetic
taacgcagcagtatcaa

SEQ ID NO: 14 - DNA, 1140 bp, Artificial Sequence
  Synthetic
tttgaggattgcggaggcggctataccccggactgcagctccaatgagaacctcacctccagcgaggaggacttctcctctggccagtccagccgcgtgtccccaagccccaccacctaccgcatgttccgggacaaaagccgctctccctcgcagaactcgcaacagtccttcgacagcagcagtccccccacgccgcagtgccataagcggcaccggcactgcccggttgtcgtgtccgaggccaccatcgtgggcgtccgcaagaccgggcagatctggcccaacgatggcgagggcgccttccatggagacgcagatggctcgttcggaacaccacctggatacggctgcgctgcagaccgggcagaggagcagcgccggcaccaagatgggctgccctacattgatgactcgccctcctcatcgccccaccggcagctgctgaaggacagcttcatggtggagctggtggagggggcccgcaagctgcgccacgtcttcctgttcaccgacctgcttctctgcaccaagctcaagaagcagagcggaggcaaaacgcagcagtatgactgcaaatggtacattccgctcacggatctcagcttccagatggtggatgaactggaggcagtgcccaacatccccctggtgcccgatgaggagctggacgctttgaagatcaagatctcccagatcaagagtgacatccagagagagaagagggcgaacaagggcagcaaggctacggagaggctgaagaagaagctgtcggagcaggagtcactgctgctgcttatgtctcccagcatggccttcagggtgcacagccgcaacggcaagagttacacgttcctgatctcctctgactatgagcgtgcagagtggagggagaacatccgggagcagcagaagaagtgtttcagaagcttctccctgacatccgtggagctgcagatgctgaccaactcgtgtgtgaaactccagactgtccacagcattccgctgaccatcaacaaggaagatgatgagtctccggggctctatgggtttctgaatgtcatcgtccactcagccactggatttaagcagagttcaaaagcccttcagcggccagtagcatctgactttgagcctcag